(19) United States
(12) Patent Application Publication
(10) Pub. No.: US 2024/0425563 A1
(43) Pub. Date: Dec. 26, 2024

(54) MHC-INDEPENDENT TCRs AND METHODS OF MAKING AND USING SAME

(71) Applicant: Washington University, St. Louis, MO (US)

(72) Inventors: Nathan Singh, St. Louis, MO (US); Ju Fang Chang, St. Louis, MO (US)

(73) Assignee: Washington University, St. Louis, MO (US)

(21) Appl. No.: 18/261,483

(22) PCT Filed: Jan. 13, 2022
(86) PCT No.: PCT/US2022/012363

§ 371 Date: Jul. 14, 2023

Related U.S. Application Data
(60) Provisional application No. 63/248,105, filed on Sep. 24, 2021.
(60) Provisional application No. 63/136,702, filed on Jan. 13, 2021.

(51) Int. Cl. C07K 14/725; A61K 35/00; C07K 16/28

(52) U.S. Cl. CPC C07K 14/7051; A61K 35/00; C07K 16/2803; C07K 2317/51; C07K 2317/515; C07K 2317/55; C07K 2317/73

(57) ABSTRACT

Among the various aspects of the present disclosure is the provision of MHC-independent T cell receptor (miTCR) compositions and methods of making and using the same.

### CROSS-REFERENCE TO RELATED APPLICATIONS

This application is a national stage entry of PCT International Application No. PCT/US2022/012363 filed on 13 Jan. 2022, which claims the benefit of priority to U.S. Provisional Application Ser. No. 63/136,702 filed on 13 Jan. 2021, and to U.S. Provisional Application Ser. No. 63/248,105 filed on 24 Sep. 2021, which are incorporated herein by reference in their entireties.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention (file name “019636-WO_Sequence_Listing_ST25.txt” created on 13 Jan. 2022, 176,408 bytes). The subject matter of the Sequence Listing is incorporated herein by reference in its entirety.

### FIELD

The present disclosure generally relates to constructs for use in cancer therapy.

### SUMMARY

Among the various aspects of the present disclosure is the provision of MHC-independent T cell receptor (miTCR) compositions and methods. An aspect of the present disclosure provides for a construct comprising or a construct encoding an engineered chimeric MHC-independent T cell receptor (miTCR), the miTCR comprising: A) a first peptide chain comprising i) a first TCR subunit comprising a constant region alpha (C_{α}) (SEQ ID NO: 49) or a functional fragment or a functional variant thereof, and ii) a first antigen recognition variable chain comprising an antigen recognition heavy chain (V_{H}) or an antigen recognition light chain (V_{L}) or a functional fragment or functional variant thereof; and B) a second peptide chain comprising i) a second TCR subunit comprising a constant region beta (C_{β}) (SEQ ID NO: 50) or a functional fragment or a functional variant thereof; and ii) a second antigen recognition variable chain comprising an antigen recognition heavy chain (V_{H}) or an antigen recognition light chain (V_{L}) or a functional fragment or functional variant thereof; wherein, the first peptide and second peptide are capable of self assembling into a miTCR pair when expressed on a cell; the miTCR pair has an interface between the constant and variable regions; and the miTCR pair comprises an antigen recognition domain comprising the V_{H} and the V_{L} that together are capable of specifically binding to a target antigen. In some embodiments, the construct comprises V_{L}C_{α} and V_{H}C_{β} or V_{H}C_{α} and V_{L}C_{β}. In some embodiments, the functional fragment or functional variant of C_{α} (SEQ ID NO: 49) is at least 80% identical to SEQ ID NO: 49 or is at least 80% identical to a functional fragment of SEQ ID NO: 49; or the functional fragment or functional variant of C_{β} (SEQ ID NO: 50) is at least 80% identical to SEQ ID NO: 50 or is at least 80% identical to a functional fragment of SEQ ID NO: 50. In some embodiments, the functional fragment or functional variant of C_{α} (SEQ ID NO: 49) is selected from one or more of the group selected from a substitution, insertion, deletion, conservative substitution, PD/PY/PE insertion prior to residue 1 of SEQ ID NO: 49, cysteine modification to promote disulfide bonding (e.g., T47C), or combinations thereof; or the functional fragment or functional variant of C_{β} (SEQ ID NO: 50) is selected from one or more of the group selected from a substitution, insertion, deletion, conservative substitution, cysteine modification to promote disulfide bonding (e.g., S56C), or combinations thereof. In some embodiments, the C_{α} and/or C_{β} has one or more amino acids mutated to cysteine to promote disulfide bond formation, promoting surface stability of miTCR paired chains. In some embodiments, the first peptide chain or second peptide chain comprises human TCR constant regions. In some embodiments, the C_{α} chain is at least 80% identical to a functional fragment or portion of SEQ ID NO: 49. In some embodiments, the C_{β} chain is at least 80% identical to a functional fragment or portion of SEQ ID NO: 50. In some embodiments, the miTCR comprises a nucleic acid or amino acid sequence modification to the C_{α} to relieve a stressed conformation, comprising a PD, PE, or PY insertion at the beginning of the Ca; or the variant is a mutation in the constant chain of SEQ ID NO: 49, is selected from: an insertion of PD, PE, or PY before residue 1 of SEQ ID NO: 49. In some embodiments, the first TCR subunit is a C_{α} chain, and the amino acid at position 47 of the constant region (SEQ ID NO: 49) is mutated to cysteine; and the second TCR subunit is a C_{β} chain, and the amino acid at position 56 of the constant region (SEQ ID NO: 50) is mutated to cysteine. In some embodiments, V_{H} or V_{L} or both are modified to increase stability of the miTCR, in V_{H} or V_{L} residues interacting with H39, E41, Y100 of SEQ ID NO: 50 or interacting with 47V residue of SEQ ID NO: 49. In some embodiments, the antigen recognition domain comprises an immunoglobulin molecule variable heavy (V_{H}) chain fragment and an immunoglobulin molecule variable light (V_{L}) chain fragment. In some embodiments, the V_{H} is an antibody heavy chain and the V_{L} is an antibody light chain. In some embodiments, a residue from a V_{H} or V_{L} is substituted with a residue having similar chemical properties selected from: hydrophobic side chain substitution: L, I, M, V; electrically charged side chain substitution: R, K, Q; an aromatic side chain substitution: Y, F, H, W, and combinations thereof. In some embodiments, the target antigen is a tumor-specific antigen or a virus-specific antigen. In some embodiments, the miTCR is subjected to nucleic acid or amino acid sequence modification to the V_{H}, V_{L}, or both, to mimic the interface of the endogenous TCR constant and TCR variable regions comprising replacing amino acids of V_{H} and V_{L} of the antigen recognition domain with amino acids corresponding to the V_{H} and V_{L} of the endogenous TCR and mutation positions and amino acids are selected from corresponding native TCR variable heavy or variable light chain residues or a conservative substitution thereof, resulting in improved interaction in the interface. In some embodiments, the miTCR is subjected to nucleic acid or amino acid sequence modification to the V_{H}, V_{L}, or both, to at least partially restore the hydrophobic pockets or hydrogen bond formation endogenous to the interface between the constant and variable regions in wild type TCR. In some embodiments, the miTCR is subjected to nucleic acid or amino acid sequence modification to the V_{H}, V_{L}, or both, to result in a mutated interface, wherein the mutated interface is more hydrophobic compared to the interface of wild type antigen recognition domain and wild type TCR constant domain. In some embodiments, the miTCR is subjected to nucleic acid or amino acid sequence modification to the V_{H}, V_{L}, or both, to result in a mutated interface, wherein the mutated interface recreates the hydrogen bonding between the variable regions and constant regions of wild type TCR. In some embodiments, the miTCR is subjected to nucleic acid or amino acid sequence modification to the V_{H}, V_{L}, or both, result in increased protein stability. In some embodiments, the antigen recognition domain targets antigens expressed on a cancer cell, wherein the cancer expressing the antigen is selected from the group consisting of adrenal cortex cancer, bladder cancer, breast cancer, cervical cancer, bile duct cancer, colorectal cancer, and esophageal cancer, Glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, Plasmacytoma, neuroblastoma, ovarian cancer, prostate cancer, sarcoma, gastric cancer, uterine cancer, and thyroid cancer; or, the viral infection is caused by a virus selected from the group consisting of cytomegalovirus (CMV), Epstein-Barr Virus (EBV), Hepatitis B virus (HBV), Kaposi's sarcoma-associated herpes virus (KSHV), human papilloma virus (HPV), molluscum virus (MCV), human T-cell leukemia virus 1 (HTLV-1), HIV (human immunodeficiency virus), and hepatitis C virus (HCV). In some embodiments, the antigen recognition domain targets a target antigen or tumor associated protein selected from the group consisting of CD19, CD20, EGFR, Her2, PSCA, CD123, CEA (Carcinoembryonic Antigen), FAP, CD133, EGFRVIII, BCMA, PSMA, CA125, EphA2, C-met, L1CAM, VEGFR, CS1, ROR1, EC, NY-ESO-1, MUC1, MUC16, mesothelin, LewisY, GPC3, GD2, EPG, DLL3, CD99, 5T4, CD22, CD30, CD33, CD138, or CD171. In some embodiments, the antigen recognition domain is derived from or comprises a functional portion, fragment, or mutant of an antibody, antibody heavy chain variable region, or antibody light chain variable region derived from IMCC225 (cetuximab, Cetuximab/Cetux), Ofatumumab (Ofalimumab), CD19 monoclonal antibody FMC63, Rituximab (Melohua), Avastin (Bevacizumab), BEC2 (Atumolimumab), Bexxar (Tosimolimumab), Campath (Alenzumab), Herceptin (Trastuzumab), LymphoCide (Epazumab), MDX-210, Mylotarg (Jimzumab Ozomicin), mAb 17-1A (Edrugrol Monoclonal antibody), Theragyn (pemtumomab), Zamyl, Zevalin (teimumab) or high affinity antibodies obtained by screening. In some embodiments, the variable heavy (V_{H}) chain fragment and the variable light (V_{L}) chain fragment are functional fragments or mutants of heavy chains and light chains targeting CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-11 Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-1 receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTI); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 1 B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL1 1Ra, IL13Ra2, CD179b-IGLL1, ALK TCR gamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLVI-Tax, CMV pp65, EBV-EBNA3c, influenza A hemagglutinin (HA), GAD, PDL1, Guanylyl cyclase C (GCC), KSHV-K8.1 protein, KSHV-gH protein, auto antibody to desmoglein 3 (Dsg3), autoantibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IGE, CD99, RAS G12V, Tissue Factor 1 (TF1), AFP, GPRC5D, claudin 18.2 (CLD18A2 OR CLDN18A.2)), P-glycoprotein, STEAP1, LIV1, NECTIN-4, CRIPTO, GPA33, BST1/CD157, low conductance chloride channel, and antigen recognized by TNT antibody. In some embodiments, the miTCR comprises a mutation in the V_{L} chain of SEQ ID NO: 35, selected from: Q101P, Q101L, Q101A, Q101G, Q101I, or Q101V; or L36E, L36D, L36K, L36R, L36N, or L36Q, or combinations thereof. In some embodiments, the variant is a mutation in the V_{H} of SEQ ID NO: 36, selected from: G31K, G31R, or G31Q; or P32F, P32L, P32V, P321, or P32A, or combinations thereof. In some embodiments, the variant is a mutation in the V_{H} chain of SEQ ID NO: 36 is selected from T108L, T1081, T108M, or T108V or combinations thereof. In some embodiments, the variant is a mutation in the V_{H} chain of SEQ ID NO: 35, is selected from: S30K, S30R, S30H, or S31Q; or S31F, S31Y, S31H, or S31W or combinations thereof. In some embodiments, the miTCR is selected from: (1) FMC63 light chain variable region and a TCRα constant region; (2) FMC63 heavy chain variable region and a TCRα constant region; (3) FMC63 light chain variable region TCRβ constant region; or (4) FMC63 heavy chain variable region TCRβ constant region. In some embodiments, the at least 80% identical functional fragment or portion is operably linked to an antigen recognition domain and has antigen binding or cytotoxic activity. In some embodiments, the construct is selected from any one of: miTCR1_α_wt_β_G31K_P32F; miTCR1_α_PD_β_wt; miTCR1_α_PD_β_G31K_P32F; miTCR1_α_Q101L_β_wt; miTCR1_α_PD_Q101L_β_wt; miTCR1_α_PD_Q101L_L36E_β_wt; miTCR1_α_Q101L_β_G31K_P32F; miTCR1_α_PD_Q101L_β_G31K_P32F; miTCR1_α_PD_Q101L_L36E_β_G31K_P32F; miTCR2_α_wt_β_S32K_S33F; miTCR2_α_S141PD_β_wt; miTCR2_α_S141PD_β_S32K_S33F; miTCR2_α_S141PD_T108L_β_wt; miTCR2_α_S141 PD_T108L_β_S32K_S33F; conservative substitutions thereof; or a sequence at least 80% identical thereof having CD19 binding activity. In some embodiments, the construct further comprising a costimulating domain selected from CD80(CD86)/CD28, 4-1 BBL/4-1BBL, CD27, OX-40, ICOS, SLAM, CD84, CD30, GITR, CD40L, or combinations thereof. In some embodiments, the miTCR has antigen binding activity. In some embodiments, residues located at the interface for the V_{H} and V_{L} of the antigen recognition domain having contact with the interface residues from TCR constant region are modified or mutated to mimic the hydrophobic core properties of a native TCR interface. In some embodiments, the miTCR kills cancer cells or reduces cancer cell proliferation at least as well or better than a CAR having the same antigen recognition domain. In some embodiments, the miTCR variant kills cancer cells or reduces cancer cell proliferation at least as well or better than wild type miTCR having the same antigen recognition domain. In some embodiments, the V_{H} or V_{L} mutations increases surface expression on a cell compared to an unmutated antigen recognition domain. In some embodiments, the variant comprises a mutation that increases cytotoxic activity or function compared to an unmutated antigen recognition region. In some embodiments, the miTCR is subjected to nucleic acid or amino acid sequence modification to enhance protein stability and increase expression of the miTCR. In some embodiments, the miTCR is subjected to nucleic acid or amino acid sequence modification in Ca, Cr, V_{H}, or V_{L} to enhance cytotoxicity of a cell expressing the miTCR. In some embodiments, the miTCR comprises mutations that alleviate amino acid intrusion into hydrophobic pocket, improves surface expression, or improves receptor-driven T cell function. In some embodiments, the miTCR comprises a mutation that improves the interaction or orientation of the variable region and constant region interface. In some embodiments, the miTCR or miTCR pair are capable of integrating the antigen recognition domain comprising immunoglobulin variable chains into an endogenous TCR complex. In some embodiments, the miTCR pair preserves the highly-regulated TCR complex-driven cellular activation and integrates MHC-independent antigen recognition. In some embodiments, the miTCR pairs or plasmid sequence comprises variable heavy or variable light chains with TCR alpha or beta constant regions to create a single chain antigen receptor. In some embodiments, each alpha chain pairs with a beta chain to generate a complete TCR, which self-assembles with associated CD3 chains during intracellular packaging. In some embodiments, the first peptide chain and second peptide chain are capable of self-assembling into a miTCR pair when expressed in a cell. Another aspect of the present disclosure provides for a construct encoding an engineered chimeric miTCR of any one of the preceding embodiments, comprising a flanking sequence to increase protein expression (optionally, gtcgacgttaacgccgccacc) preceding the TCR constant beta chain sequence and/or aagcggccgc at the end of the DNA sequence. In some embodiments, he construct encoding an engineered chimeric miTCR comprises a plasmid comprising a La plasmid (promoter+V_{L}C_{α}) and a HR plasmid (promoter+V_{H}C_{β}); or an Ha plasmid (promoter+V_{H}C_{α}) and a LP plasmid (promoter+V_{L}C_{β}). In some embodiments, the C_{α} is optionally linked to the CR chain. In some embodiments, the construct encodes a self-cleaving peptide or a ribosomal skipping sequence (e.g., furin-GSG-T2A, F2A, P2A, E2A) between the alpha and beta chains to prevent covalently linking the miTCR pair. In some embodiments, the first peptide and second peptide are expressed in a two-vector system, wherein no linker and no cleavage is necessary. In some embodiments, the construct further encodes a costimulating domain selected from CD80(CD86)/CD28, 4-1BBL/4-1BBL, CD27, OX-40, ICOS, SLAM, CD84, CD30, GITR, CD40L, or combinations thereof. Another aspect of the present disclosure provides for a nucleic acid encoding the polypeptide sequence selected from any one of SEQ ID NO: 19-32; conservative substitutions thereof; or a sequence at least 80% identical thereof, having CD19 binding activity. Another aspect of the present disclosure provides for a nucleic acid sequence, or comprising a nucleic acid selected from any one of SEQ ID NO: 3-16; conservative substitutions thereof; or a sequence at least 80% identical thereof, having CD19 binding activity. Another aspect of the present disclosure provides for a vector comprising the miTCR according to any one of the preceding embodiments, optionally, a lentiviral vector. Another aspect of the present disclosure provides for a composition comprising a miTCR construct of any one of the preceding embodiments or a plasmid sequence directed against a surface molecule/antigen (e.g., CD19). In some embodiments, the surface molecule/antigen is CD19 and the surface is the surface of a CD19+ leukemia cell. In some embodiments, the plasmid sequence comprises a sequence resulting in the translation of an antigen recognition domain (e.g., FMC63 anti-CD19 domain). Another aspect of the present disclosure provides for a method of treating a target antigen-related disease in a subject in need thereof comprising a construct of any one of the preceding embodiments. In some embodiments, the target antigen-related disease is a cancer or virus infection-related disease. In some embodiments, the disease associated with expression of a disease-associated antigen selected from the group consisting of a proliferative disease, a precancerous condition, a cancer, and a non-cancer related indication associated with expression of the disease-associated antigen. In some embodiments, the antigen-related disease is cancer selected from the group consisting of adrenocortical cancer, bladder cancer, breast cancer, cervical cancer, bile duct cancer, colorectal cancer, esophageal cancer, and glioblastoma, Glioma, hepatocellular carcinoma, head and neck cancer, kidney cancer, lymphoma, leukemia, lung cancer, melanoma, mesothelioma, multiple myeloma, pancreatic cancer, pheochromocytoma, plasmacytoma, nerve Blastoma, ovarian cancer, prostate cancer, sarcoma, gastric cancer, uterine cancer, and thyroid cancer; or, the viral infection is caused by a virus selected from the group consisting of: cytomegalovirus (CMV), E-Padii virus (Epstein-BarrVirus; EBV), Hepatitis B virus (HBV), Kaposi's sarcoma-associated herpes virus (KSHV), human papilloma virus (HPV), molluscum virus (MCV), human T cell leukemia virus 1 (HTLV-1), HIV (human immunodeficiency virus), and hepatitis C virus (HCV). In some embodiments, the antigen-related disease is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, primary effusion lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia. In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, Merkel cell cancer, epidermoid cancer, squamous cell cancer, T-cell lymphoma, environmentally induced cancers, combinations of said cancers, and metastatic lesions of said cancers. In some embodiments, treating a subject using miTCRs do not result in classical CAR T toxicity. Another aspect of the present disclosure provides for an effector cell expressing the miTCR of any one of the preceding embodiments on its cell surface. In some embodiments, the effector cell is a lymphocyte, a T cell, a cytotoxic T cell, a memory T cell, a helper T cell, a natural killer T cell, iNKT cells, NK cells, memory like NK cells, or a regulatory or suppressor T cell. Another aspect of the present disclosure provides for a complex formed by the miTCR pair that specifically binds to a target antigen according to any one of the preceding embodiments. Another aspect of the present disclosure provides for a miTCR, a complex, a nucleic acid, or the effector cell according to any one of the preceding embodiments, for use in preparing a kit, or a preparation. Another aspect of the present disclosure provides for a pharmaceutical composition comprising the miTCR according to any one of the preceding embodiments, the complex according to any one of the preceding embodiments, the nucleic acid according to, the vector of any one of the preceding embodiments, or the effector cell of any one of the preceding embodiments, and a pharmaceutically acceptable carrier. Another aspect of the present disclosure provides for a method for killing a target cell presenting a target antigen, comprising contacting the target cell with the effector cell of any one of the preceding embodiments, wherein the miTCR specifically binds to the target antigen. Another aspect of the present disclosure provides for a method of treating a subject having cancer comprising administering to a subject in need thereof a therapeutically effective amount of a pharmaceutical composition or pharmaceutical composition made according to any one of the preceding embodiments. Another aspect of the present disclosure provides for a method for treating a target antigen-related disease or a cancer or virus infection-related disease in a subject in need thereof, comprising administering to the subject an effective amount of a pharmaceutical composition, the pharmaceutical composition comprising the construct according to any one of the preceding embodiments, a complex comprising the construct according to any one of the preceding embodiments, a nucleic acid according to any one of the preceding embodiments, a vector according to any one of the preceding embodiments, an effector cell of any one of the preceding embodiments, or a pharmaceutical composition according to any one of the preceding embodiments. Another aspect of the present disclosure provides for a cell therapy product comprising a construct of any one of the preceding embodiments. In some embodiments, the cell therapy product has T cell-based anti-tumor activity or the construct of any one of the preceding embodiments, having the highly-organized and precise signaling of an endogenous TCR complex and MHC-independent antigen recognition. In some embodiments, the cell therapy product has anti-tumor function and reduced CART toxicity. In some embodiments, the cell therapy is an allogenic cell therapy product. In some embodiments, the cell therapy is an autologous cell therapy product. Another aspect of the present disclosure provides for a method of making cell therapy product comprising: providing a lymphocyte cell; removing endogenous TCR alpha and beta chains or disrupting expression of endogenous TCR alpha and beta chains, optionally, knocking out via CRISPR; introducing a vector comprising an miTCR construct of any one of the preceding embodiments or expressing an miTCR construct of any one of the preceding embodiments on the lymphocyte cell. Another aspect of the present disclosure provides for a method to create engineered miTCR T cells comprising: engineering T cells expressing chimeric miTCRs receptors and CRISPR editing to knock out endogenous TCR alpha and beta chains. Another aspect of the present disclosure provides for a method of generating an miTCR comprising: the use of lentiviral-based T cell engineering to express the miTCRs of any one of the preceding embodiments; and CRISPR-based gene editing to disrupt expression endogenous TCR alpha and beta chains. In some embodiments, the gene editing efficiency is greater than about 98%. In some embodiments, nearly all translated alpha and beta TCR chains pair appropriately. In some embodiments, the method further comprising purifying a cell population using a selection marker to 100% engineered cells. Another aspect of the present disclosure provides for a method of detecting interface regions that need to be modified to more closely match native TCR folding comprising: constructing a model of a native TCR constant region; constructing a model of a native variable antigen recognition domain; constructing a model of the interface of the TCR constant region and antigen recognition domain variable region; and comparing the interface of the TCR constant region and antigen recognition domain variable region to the interface of native TCR interface; and determining desired mutations or modifications to the amino acid sequence based on reducing clashes that disrupt hydrogen bonding or disrupt a native hydrophobic pocket or core. Another aspect of the present disclosure provides for a construct comprising or a construct encoding an engineered chimeric MHC-independent T cell receptor (miTCR), the miTCR comprising: A) a first peptide chain comprising i) a first TCR subunit comprising a constant region alpha (C_{α}) (SEQ ID NO: 49) or a functional fragment or a functional variant thereof; and ii) a first antigen recognition variable chain comprising an antigen recognition fragment or a functional fragment or functional variant thereof; and B) a second peptide chain comprising i) a second TCR subunit comprising a constant region beta (C_{β}) (SEQ ID NO: 50) or a functional fragment or a functional variant thereof; and ii) a second antigen recognition variable chain comprising an antigen recognition fragment or a functional fragment or functional variant thereof; wherein, the first peptide and second peptide are capable of self assembling into a miTCR pair when expressed on a cell, the miTCR pair comprising an antigen recognition domain comprising the an antigen recognition fragment that together are capable of specifically binding to a target antigen. In some embodiments, the antigen recognition fragments are selected from V_{H} and V_{L} regions, antigen-recognition fragments that are comprised of protein ligands, simple peptides, camelids, individual variable chains, or KIRs (killer cell immunoglobulin-like receptors).

Other objects and features will be in part apparent and in part pointed out hereinafter.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1. Crystal structure of the immunoglobulin antigen-binding fragment (Fab), composed of the variable and constant region 1 domains of both heavy and light chains. See also FIG. 14.

FIG. 2. Cryo-EM structure of the TCR complex, including the alpha and beta chains as well associated CD3 chains (top), and Cryo-EM structure of the alpha and beta chains in isolation (bottom).

FIG. 3. miTCR synthesis strategy. Immunoglobulin Fab and TCR alpha and beta chains responsible for antigen binding (top). Predicted structures of miTCR alpha and beta chains demonstrating that these hybrid proteins are predicted to adopt highly similar structures to their endogenous counterparts (bottom).

FIG. 4. Schematics of miTCR alpha and beta chain expression plasmids using a two vector system. The one vector system is shown in FIG. 19A-FIG. 19C.

FIG. 5. Flow cytometric evaluation of Jurkat cell line containing an NFAT-GFP reporter. Jurkats were evaluated after 18 hour co-incubation with either no target or with CD19+ leukemia. Jurkats without a synthetic receptor (top). Jurkats bearing a CD19-targeted CAR (labeled with mCherry, bottom).

FIG. 6. Flow cytometric evaluation of Jurkat cell line containing an NFAT-GFP reporter. Jurkats were evaluated after 18 hour co-incubation with either no target or with CD19+ leukemia. Jurkat expression of miTCR chains (left). NFAT-GFP expression in Jurkats expressing only the beta chain, alpha and beta chains, only the alpha chain, or neither, demonstrating potent NFAT activation only with expression of both chains (right, clockwise from top left).

FIG. 7A-FIG. 7D. (a) Jurkat cells without vector; (b) Jurkat cells electroporated with a vector expressing an endogenous TCR targeting the cancer antigen NYESO (1G4); (c) Jurkat cells electroporated with a vector expressing an miTCR1 (vL-Cα+vH-Cβ); (d) Jurkat cells electroporated with a vector expressing an miTCR2 (vH-Cα+vL-Cβ).

FIG. 8A-FIG. 8C. (a) wild type TCR alpha chain based on electron microscopy structure 6JXR; (b) predicted structure of wild type miTCR1 alpha chain identifying disruption with hydrophobic pocket (intrusion of Q101), lack of hydrogen bonding between K185 and E100, other variable region residues resulting in a stressed conformation; (c) different view of conflict between Q101 and 1129. Comparison of the protein structure of the interface of native TCR (a) and miTCR1 (b) shows that the stable hydrophobic core of native TCR is disrupted in miTCR1 due to the Q101 residue in the variable domain.

FIG. 9A-FIG. 9D. (a) mutation of Q101P which alleviates intrusion into hydrophobic pocket; (b) mutation of Q101L which alleviates intrusion into hydrophobic pocket and improves surface expression over Q; predicted structure is still stressed, tight hydrophobic pocket, no hydrogen bonds; (c) insertion of PD prior to the beginning of the TCR constant chain, opening up pocket; this recreates the hydrogen bonds between K185 and E100 and alleviate the stressed conformation (d) mutation of L36E, recreating hydrogen bonds between K185 and L36E.

FIG. 10A-FIG. 10C. (a) wild type TCR beta chain based on electron microscopy structure 6JXR; (b) lack of a stabilizing aromatic residue on variable chain and the lack of hydrogen bond between E184 and its neighboring residues; (c) introduction of aromatic residue in variable chain and mutation of G31K on variable chain to introduce a hydrogen bond.

FIG. 11A-FIG. 11C. (a) wild type miTCR2 alpha chain identifying lack of hydrophobic core due to intrusion of polar residues T108 and S141; (b) mutation of S into PD (S141 PD), opening up pocket and relaxing constant chain; (c) mutation of T108L, stabilizing hydrophobic pocket.

FIG. 12A-FIG. 12B. (a) wild type miTCR2 beta chain identifying lack of stabilizing aromatic residue and hydrogen bond between E184 and its neighboring residues; (b) mutation of S32K and S33F to stabilize constant chain aromatic residues and inter-chain hydrogen bonds.

FIG. 13A-FIG. 13B. (a) schematic and (b) model of structure of the endogenous T cell receptor (TCR) with associated CD3 molecules.

FIG. 14. Structure of the endogenous B cell receptor (immunoglobulin).

FIG. 15. Design scheme of MHC-independent TCRs (miTCR), in which the antigen-binding region of the TCR is replaced with that from the BCR.

FIG. 16A-FIG. 16B. Predicted structure of (a) miTCR1 and (b) miTCR2.

FIG. 17A-FIG. 17B. (a) schematic of native TCR (1 G4) construct as well as miTCR1 and miTCR2 constructs without codon optimization and in beta-alpha order; (b) flow cytometry of Jurkat TCR knockout cells transfected with the constructs shows that miTCR1 (bottom left) and miTCR2 (bottom left) are not well-expressed on the cell surface compared to 1G4 (top right).

FIG. 18A-FIG. 18B. (a) schematic of native TCR (1G4) construct as well as miTCR1 and miTCR2 constructs with codon optimization and in alpha-beta order; (b) flow cytometry of Jurkat TCR knockout cells transfected with the constructs shows that codon optimization of the miTCR constructs improves cell surface expression of miTCR1 (bottom left) and miTCR2 (bottom right).

FIG. 19A-FIG. 19D. Schematic of (a) native TCR without codon optimization (1G4), (b) schematic of (b) and (c) codon-optimized miTCR1, codon-optimized miTCR2 constructs where the F2A site is replaced with a furin-GSG-T2A site in both miTCR1 and miTCR2, (d) difference in expression of WT (1G4) vs. miTCR when Fur_F2A was used. Here, a two vector system is used.

FIG. 20. Flow cytometry of Jurkat cells transfected with the miTCR constructs in FIG. 20 shows that changing the F2A site to GSG-T2A significantly improves the surface expression of miTCR1 (bottom left) and miTCR2 (bottom right).

FIG. 21. Flow cytometry of human T cells lacking endogenous TCR and transfected with the miTCR constructs in FIG. 19A-FIG. 19C shows that miTCR1 (middle left) and miTCR2 (middle right) are efficiently expressed on the cell surface. The miTCRs were detected using an antibody against FMC63, the parental BCR used to manufacture the anti-CD19 variable chains. The upper left corner showed the 98% of knockdown efficiency of endogenous TCR (pink is TCR CRISPRed cell, blue is wild type). BB and 28 refer to CAR against CD19 with 41bbL and CD28 costimulation domain, respectively.

FIG. 22. Flow cytometry of human T cells lacking endogenous TCR and transfected with the miTCR constructs in FIG. 19A-FIG. 19C shows that miTCR1 (top right) and miTCR2 (middle left) are efficiently expressed on the cell surface. The miTCRs were detected using an antibody against the TCR constant chains.

FIG. 23. Flow cytometry of human T cells lacking endogenous TCR and transfected with the miTCR constructs in FIG. 19A-FIG. 19C shows that cells that express TCR constant chains also stain with anti-FMC63, demonstrating that both regions of the miTCRs (variable from BCR and constant from TCR) can be detected on the same cells.

FIG. 24. Flow cytometry of human T cells lacking endogenous TCR and transfected with the miTCR constructs in FIG. 19A-FIG. 19C shows that cells containing miTCR1 or miTCR2 express CD3 on the cell surface, suggesting that miTCRs form native TCR-like complexes.

FIG. 25. Flow cytometry of human T cells lacking endogenous TCR and transfected with the miTCR constructs in FIG. 19A-FIG. 19C shows that while the modifications to the constructs have improved miTCR expression, miTCRs are still not expressed on the surface as efficiently as native TCRs.

FIG. 26. Protein structure of the interface between the variable and constant regions of the miTCRs. Related to FIG. 8-FIG. 12.

FIG. 27. Protein structure of the interface of miTCR1 shows a conflict between amino acid side chains that potentially impacts protein structure or folding.

FIG. 28A-FIG. 28B. (a) The structural stress of the miTCR1 interface is predicted to be reduced by inserting a PD residue in the junction in the alpha chain. (b) Structures of native TCR, miTCR1_alpha_PD, miTCR1_alpha_PD_Q101L, and miTCR1_alpha_PD_Q101L_L36E.

FIG. 29A-FIG. 29B. Cell surface expression of miTCRs in (a) Jurkat and (b) human T cells transfected or infected with the modified miTCR constructs containing inserted or substituted residues in the interface as listed in TABLE 1-TABLE 3. Many of the modifications improved cell surface expression compared to the parent constructs.

FIG. 30. Jurkat cells containing a fluorescent reporter system and expressing miTCRs or CD28 and 41 BB-based CARs targeting CD19 with either Nalm6 (CD19+) or Molm14 (CD19-negative). The reporters were only activated with CD19, showing that miTCRs are activated in an antigen-specific manner.

FIG. 31. Parent constructs (JFC029, 039), CARs (BB and 28), and mutated constructs (JFC030, 031, 040) were combined with CD19+ Nalm6 cells and serially evaluated in co-culture for killing efficacy. All mutants evaluated improved the function of their parental counterparts.

FIG. 32. On the last time point of the killing assay (day 6), miTCRs (except for JFC039, the parent miTCR2 construct) are more effective at killing targets than TCRs (1G4), and have efficacy similar to CARs.

FIG. 33. Schematic showing a two-signal model of T cell activation.

FIG. 34A-FIG. 34B. Target leukemia cells (Nalm6) were engineered to express co-stimulatory ligands in addition to miTCR ligand CD19. miTCRs demonstrate comparable or better (a) killing and (b) proliferation compared to CARs.

### DETAILED DESCRIPTION OF THE INVENTION

The present disclosure is based, at least in part, on the discovery that identifying that the variable chain/constant chain interface influences receptor expression and function. As shown herein is the development of an MHC-independent T cell receptor (miTCR) to overcome the MHC-dependence of traditional TCRs while harnessing the power of this complex's intricate cellular activation. As shown herein, the miTCR was able to preserve the highly-regulated TCR complex-driven cellular activation and integrate MHC-independent antigen recognition.

Two of the fundamental barriers to the success of chimeric antigen receptor (CAR)-engineered T cells in the treatment of cancer are (1) toxicity in the form of cytokine release syndrome (CRS), thought to result from overly-exuberant T cell activation, and (2) impaired CAR T cell persistence, thought to result from the rapid and aberrant development of T cell dysfunction. Both of these processes are dependent on antigen-dependent CAR-driven intracellular signaling, a process that, by virtue of the simplistic CAR design, is much stronger as well as less organized than that of the native T cell antigen receptor (TCR).

While CARs are a single polypeptide chain containing antigen-binding and intracellular signaling domains, TCRs are a complex of 8 distinct polypeptides, each of which contains multiple motifs which perform an essential role in directing T cell activation upon antigen recognition. TCRs are capable of directing potent T cell activation and effector function while preventing the development of T cell dysfunction (in the absence of persistent antigen exposure) and enabling the development of long-lived T cell memory. This powerful and versatile function results from tightly-regulated TCR complex signaling. Transgenic TCRs (tTCRs) are synthetic alpha and beta TCR chains that recognize cancer antigens presented by class-restricted MHC molecules. While tTCRs have demonstrated some clinical efficacy, this platform is significantly limited by (1) the requirement for MHC matching for each individual TCR, greatly reducing the number of patients eligible for this therapy, and (2) the routine down-regulation of MHC molecules in many cancers.

MHC-Independent T Cell Receptor (miTCR)

Chimeric antigen receptor (CAR) T cells can result in a cure for a small fraction of patients with B cell malignancies, however, CART cells are often limited by lack of effective disease response or lack of maintained disease response, reflecting a failure of T cell persistence and memory formation. Even in cases of CART success, many patients also develop cytokine release syndrome (CRS), a life-threatening hyper-inflammatory state driven by uncontrolled T cell activation.

To overcome these barriers that result in unregulated CAR-driven T cell signaling while maintaining the power of the T cell immune response, here is described the development of MHC-independent TCRs (miTCRs). These are synthetic TCR alpha and beta chains that integrate the antigen-recognition motif employed by CARs (immunoglobulin variable chains) into the endogenous TCR complex. These MHC-independent TCRs (miTCRs) overcome the MHC-dependence of traditional TCRs while harnessing the power of this complex's intricate cellular activation.

The miTCRs as described herein comprise completely human TCR alpha and beta constant region sequences, except slightly modified to make the TCR alpha and beta constant regions form disulfide bonds to stabilize the pairing. These “C” mutations are enlarged and bolded in the protein sequence listings. For the alpha chain: TDK “T” VLDM was mutated to TDK “C” VLDM and for the beta chain: HSGV “S” TDP was mutated to HSGV “C” TDP.

Each chain has one single C mutation. The T and S are believed to likely be the only two residues in the WT constant chain that would be useful to be mutated into C, and other C modifications can be made.

WT alpha chain sequence without the T47C modification is (SEQ ID NO: 49):

IQNPDPAVYQLRDSKSSDKSVCLFTDFDSQTNVSQSKDSDVYITDKTVL
DMRSMDFKSNSAVAWSNKSDFACANAFNNSIIPEDTFFPSPESSCDVKL
VEKSFETDTNLNFQNLSVIGFRILLLKVAGFNLLMTLRLWSS

The 47V residue, enlarged and double-underlined, of SEQ ID NO: 49 was identified to have interface contact with a variable region.

WT beta chain sequence without the S56C modification is (SEQ ID NO: 50):

DLKNVFPPEVAVFEPSEAEISHTQKATLVCLATGFYPDHVELSWWVNGK
EVHSGVSTDPQPLKEQPALNDSRYCLSSRLRVSATFWQNPRNHERCQVQ
FYGLSENDEWTQDRAKPVTQIVSAEAWGRADCGFTSESYQQGVLSATIL
YEILLGKATLYAVLVSALVLMAMVKRKDSRG

H39, E41, Y100 of SEQ ID NO: 50, enlarged and double-underlined are regions identified to have interface contact with a variable region.

As described herein, an MHC-independent T cell receptor has been developed. By replacing the antigen-recognition domains of the TCR (alpha and beta variable chains) with the antigen-recognition domains of the immunoglobulin molecule (variable heavy (V_{H}) and light chains (V_{L})), the miTCR was able to preserve the highly-regulated TCR complex-driven cellular activation and integrate MHC-independent antigen recognition.

This approach unleashes the full potential of T cell-based anti-tumor activity using the highly-organized and precise signaling of the endogenous TCR complex while enabling an MHC-independent antigen recognition platform. This technology can result in more successful and sustained anti-tumor function while averting classical CAR T toxicity.

Methods of previous work use murine TCR because human cells would not express the TCRs efficiently. It was not previously thought to use CRISPR to remove endogenous TCR to prevent TCR mismatching.

### Antigen Binding Domain

Antigen recognition/binding fragments/domains can comprise variable light chain and heavy chain regions incorporated in the miTCR.

As described herein, the miTCR pair can comprise an antigen binding domain or tumor binding domain. The antigen binding domain can comprise any domain that binds to an antigen expressed by the targeted cell type (e.g., an antigen, such as CD19). The TCR antigen binding domain can bind to or be derived from the variable regions targeting one or more of disease-associated antigens selected from a group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD 117); Interleukin-13 receptor subunit alpha-2 (IL-13Ra2 or CD213A2); Mesothelin; Interleukin 11 receptor alpha (IL-11 Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-1 receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WTI); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin BI; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen) Fucosyl-GM1, PTK7, gpNMB, CDH1-CD324, DLL3, CD276/B7H3, IL1 1Ra, IL13Ra2, CD179b-IGLL1, ALK TCR gamma-delta, NKG2D, CD32 (FCGR2A), Tn ag, CSPG4-HMW-MAA, Tim1-/HVCR1, CSF2RA (GM-CSFR-alpha), TGFbetaR2, VEGFR2/KDR, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Chorionic Gonadotropin Hormone receptor (CGHR), CCR4, GD3, SLAMF6, SLAMF4, HIV1 envelope glycoprotein, HTLVI-Tax, CMV pp65, EBV-EBNA3c, influenza A hemagglutinin (HA), GAD, PDL1, Guanylyl cyclase C (GCC), KSHV-K8.1 protein, KSHV-gH protein, auto antibody to desmoglein 3 (Dsg3), autoantibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IGE, CD99, RAS G12V, Tissue Factor 1 (TF1), AFP, GPRC5D, claudin 18.2 (CLD18A2 OR CLDN18A.2)), P-glycoprotein, STEAP1, LIV1, NECTIN-4, CRIPTO, GPA33, BST1/CD157, low conductance chloride channel, and antigen recognized by TNT antibody.

Other antigen binding fragments to be used in constructing an antigen recognition domain that could be implemented using the Cα Cβ TCR platform can be antigen-recognition fragments that are comprised of protein ligands, simple peptides, camelids, individual variable chains, or KIRs (killer cell immunoglobulin-like receptors).

As another example, the TCR antigen binding domain(s) can comprise an antibody, an antibody fragment, an scFv, a Fv, a Fab, a (Fab′)2, a single domain antibody (SDAB), a V_{H} or V_{L} domain, a camelid vHH domain, a non-immunoglobulin antigen binding scaffolds such as DARPINs, affibodies, affilins, adnectins, affitins, obodies, repebodies, fynomers, alphabodies, avimers, atrimers, centyrins, pronectins, anticalins, kunitz domains, Armadillo repeat proteins, a receptor or a ligand. As another example, the TCR antigen binding domain can be an antibody or functional portion thereof, such as V_{H} and V_{L} regions (from human, mouse, or other animal), a humanized antibody, a monoclonal antibody, a polyclonal antibody, a synthetic antibody, a camelid antibody, a native receptor or ligand, or a fragment thereof. For example, the antigen binding domain can be portions of a single-chain variable fragment (scFv) of an antibody. The antigen binding domain can be directed to various tumor associated proteins, which may include EphA2, alphafetoprotein (AFP), carcinoembryonic antigen (CEA), CA-125, MUC-1 antibody, CD19, CD20, CD123, CD22, CD30, SlamF7, CD33, EGFRvIII, BCMA, GD2, CD38, PSMA, B7H3, EPCAM, IL-13Ra2, PSCA, Mesothelin, Her2, LewisY, LewisA, CIAX, epithelial tumor antigen (ETA), tyrosinase, melanoma-associated antigen (MAGE), abnormal products of ras or p53, or other proteins found to be more highly enriched on the surface of tumor cells than critical normal tissues. Targeting antibody fragments or scFvs, as described herein, can be against any disease-associated antigen or tumor-associated antigen (TAA). A TAA can be any antigen known in the art to be associated with tumors.

scFvs are well known in the art to be used as a binding moiety in a variety of constructs (see e.g., Sentman 2014 Cancer J. 20 156-159; Guedan 2019 Mol Ther Methods Clin Dev. 12 145-156). Any scFv known in the art or generated against an antigen using means known in the art can be used as the binding moiety.

The antigen-binding capability of the miTCR is defined by the extracellular scFv. The format of a scFv is generally two variable domains linked by a flexible peptide sequence, either in the orientation VH-linker-VL or VL-linker-VH. But in the miTCRs, the VH and VL regions are not linked, but each variable region is incorporated on a separate TCR constant chain (a or P) subunit of the miTCR pair. The orientation of the variable domains within the miTCR pair, depending on the structure of the variable chains, may contribute to whether a miTCR will be expressed on the cell surface or whether the T cells target the antigen and signal. In addition, the length and/or composition of the variable domain linker can contribute to the stability or affinity of the expressed miTCR.

The scFv is traditionally a critical component of the CAR molecules, but the V_{H} and V_{L} subunits can be separated and can be carefully designed and manipulated to influence specificity and differential targeting of tumors versus normal tissues in the miTCRs as described herein.

Other antigen recognition domains can include gRNAs, nucleic acid aptamers, or any other moiety expressed on a tumor cell.

### Optimization of Interface Interactions

The antigen recognition domains comprising V_{H} and V_{L}, can be variants of native V_{H} and V_{L} regions to reduce conflict in order to more closely resemble the native TCR V_{H} and V_{L} regions such that the interface between the engineered V_{H} and V_{L} and the native TCR mimic or restore the native TCR variable and TCR constant region interface interactions or reduce stress in the protein.

The interface is composed of the surfaces of the variable region (e.g., V_{H} or V_{L}) that interacts with the constant region (e.g., C_{α} or C_{β}) on each miTCR chain that forms the pair. This concept of an interface, indeed applies to both native TCR and BCR antigen recognition domains (variable regions), and can apply to any chimeric antigen binding receptor.

In other words, this interface region can be generalizable to all V_{H} and V_{L}regions. The length, area, region, or portion of peptides that is closer in proximity to the constant region is relatively stable, while the distal part of the variable chains, which are responsible for antigen binding, are different for each variable chain corresponding to different antigen binding fragments or domains. Thus this peptide region closer to the constant chain is most likely to need modifications to reduce conflicts and enhance protein stability and expression.

Except for the PD insertion at the beginning of the TCR alpha constant chain (it is an insertion, not a mutation), all the mutations were made at the variable region (V_{L} and/or V_{H} of the antigen binding domain) to alleviate the conflict. The PD (or PY, PE, etc.) insertion at the beginning of the TCR alpha constant region greatly relieved the stressed conformation.

Even though the V_{L}, V_{H}, C_{α}, C_{β} between miTCR1 and miTCR2 are the same at protein sequence level, just a different combination with the same protein sequence. During codon optimization, their DNA level is slightly different (different DNA codon but code is for the same amino acid). The mutants will have the same DNA or amino acid sequence as their parent, except where the mutations are. The residues of the TCR constant region chain involved in the interface can be generalized to any different chimera protein. The below sequences illustrate the TCR constant region (see SEQ ID NO: 33 and SEQ ID NO: 34) that is involved in the interface interaction.

The residues from the variable region of an antibody can be more difficult to generalize, since the protein sequence from different antibody will be different (unlike TCR constant sequences, every chimera protein will be the same here), but the interface can be modeled and interface features can be identified for any antigen binding variable chain.

Since each antibody is different, the new concept here is it was discovered that there is a need to tailor each new chimera receptor to optimize the interface interaction. Tailoring these structural details can change a lot of aspects of the receptor, including protein expression and function.

The P (shown below) enlarged and bolded in the beginning of the TCR alpha constant region was discovered to be important, this is the P of the PD mutant (or PY, PE), which changes properties of the protein. This P was discovered to play an important role in interface interaction. This P can be generalized to other chimera production. Shown here, this P was placed in this chimera sequence and it was discovered to change a lot of protein properties.

TCR alpha constant region (italicized portion is SEQ ID NO: 33): Residues enlarged and double-underlined are involved in the interface interactions.

PYIQNPDPAVYQLRDSKSSDKSVCLFTDFDSQTNVSQSKDSDVYITDKC
VLDMRSMDFKSNSAVAWSNKSDFACANAFNNSIIPEDTFFPSPESSCDV
KLVEKSEETDTNLNFQNLSVIGFRILLLKVAGENLLMTLRLWSS

TCR beta constant region (SEQ ID NO: 34): enlarged and double-underlined are involved in the interface interactions.

DLKNVFPPEVAVFEPSEASISHTQKATLVCLATGFYPDHVELSWWVNGK
EVHSGVCTDPQPLKEQPALNDSRYCLSSRLRVSATFWQNPRNHFRCQVQ
FYGLSENDEWTQDRAKPVTQIVSAEAWGRADCGFTSESYQQGVLSATIL
YEILLGKATLYAVLVSALVLMAMVKRKDSRG

### Optional Costimulatory Signaling Domain

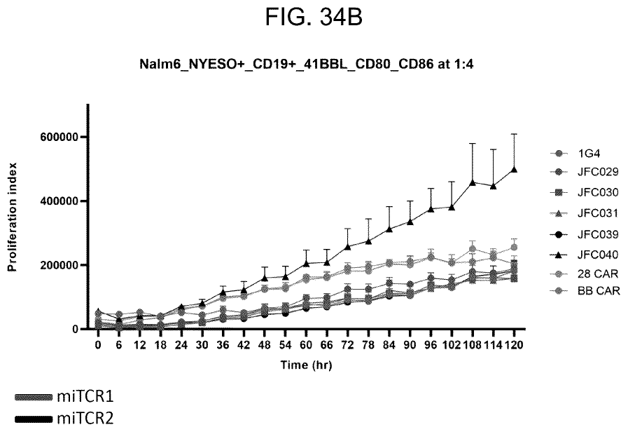

Optionally, an extracellular signaling domain or costimulatory domain or ligand (see e.g., FIG. 34) to activate T cells can be incorporated into the expression construct to propagate signaling. Costimulating domains can include CD80 (CD86)/CD28, 4-1BBL/4-1BBL, CD27, OX-40, ICOS, SLAM, CD84, CD30, GITR, CD40L, or combinations thereof.

Constructs and methods of making traditional CAR T constructs, such as extracellular and intracellular domains are well known; see e.g., Feins S, Kong W, Williams E F, Milone M C, Fraietta J A. An introduction to chimeric antigen receptor (CAR) T-cell immunotherapy for human cancer. Am J Hematol. 2019; 94(S1):S3-S9; Rafiq, S., Hackett, C. S. & Brentjens, R. J. Engineering strategies to overcome the current roadblocks in CAR T cell therapy. Nat Rev Clin Oncol 17, 147-167 (2020). Except as otherwise noted herein, therefore, the process of the present disclosure can be carried out in accordance with such processes.

Furthermore, the miTCR construct moieties can be operably linked with a linker. A linker can be any nucleotide sequence capable of linking the moieties described herein. For example, the linker can be any amino acid sequence suitable for this purpose (e.g., of a length of 9 amino acids).

### T-Cell Receptor (TCR) Subunits

### Overview of T-Cell Receptor (TCR)

The TCR is a disulfide-linked membrane-bound heterodimeric protein that is found on the surface of T cells and is responsible for recognizing peptide antigens displayed by MHC molecules. Most TCRs usually consist of highly variable α and β chains or subunits expressed as part of a complex with the invariant CD3 chain molecules. T cells expressing these two chains are referred to as αβ T cells. Besides, a small number of T cells express variable γ and α chains or subunits, called γσ T cells. The γδ TCR is comprised of variable γ and β chains expressed with CD3 on a smaller subset of T cells that recognize different types of antigens. Both of these types of receptors are expressed with a disulfide-linked homodimer of ξ chains.

In the predominant αβ TCR, each of the two disulfide-linked transmembrane α and β polypeptide chains contains one N-terminal Ig-like variable (V) domain, one Ig-like constant (C) domain, a hydrophobic transmembrane region, and a short cytoplasmic region. The V and C regions are homologous to immunoglobulin V and C regions. In the V region of each TCR chain, there are three hypervariable, or complementarity-determining regions (CDRs), named CDR1, CDR2, and CDR3, respectively. As in antibodies, CDR3 is the most variable among different TCRs.

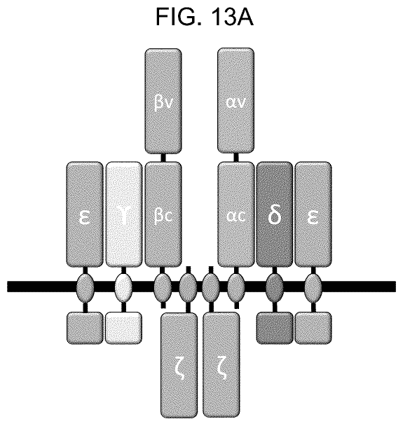

In some embodiment, the endogenous TCR alpha and beta chains (βv, βc, αc, and αv) are deleted, but for surface expression of TCR alpha and beta chains, these peptides complex with CD3 delta, CD3 epsilon, CD3 gamma, and CD3 zeta. Thus the miTCRs are using endogenous CD3 for assembly (see e.g., FIG. 13A).

### Antigen Recognition by the TCR

TCR initiates the cellular immune response by recognizing foreign peptide-MHC molecular complexes on the surface of antigen-presenting cells. Both the α and the β chain of the TCR participate in specific recognition of MHC molecules and bound peptides. Each endogenous, native, or wild-type TCR only recognizes as few as one to three residues of the MHC-associated peptide. TCR can recognize the antigen, but it cannot transmit signals to T cells on its own. Therefore, TCR recognition requires other protein complexes, called CD3 and ζ proteins, which together with TCR form the TCR complex. The CD3 and ζ chains transmit some of the signals that are initiated when the TCR recognizes an antigen. Additionally, T cell activation requires the participation of the co-receptor molecule, CD4 or CD8, which recognizes the non-polymorphic portion of the MHC molecule and is also involved in signal transduction.

Soluble TCR therapy represents a potential alternative to cell-based immunotherapies. Bispecific T cell engaging TCR can recognize tumor peptides on the cell surface. This engagement can be used to target therapeutics to this cell or engage further T cell responses.

Conventional antibody-coupled TCR consists of an extracellular domain, a transmembrane domain, a co-stimulatory signaling domain, and a TCR signaling domain. T cells containing antibody-coupled TCR can be used in combination with a variety of tumor-targeting antibodies to recognize different antigens and kill different types of tumors. When tumor-targeting antibodies bind to the surface of tumor cells, T cells containing antibody-coupled TCR can indirectly recognize tumor cells through the antibody's Fc domain, which enables the antibody-dependent cellular cytotoxicity (ADCC) for tumor cell killing.

### Cancer

Methods and compositions as described herein can be used for the prevention, treatment, or slowing the progression of cancer or tumor growth. Although the proof-of-principle example described herein is leukemia, these constructs can be useful in solid malignancies and other cancers as well. For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sézary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sézary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor. Brain or spinal cord tumors can be acoustic neuroma; astrocytoma, atypical teratoid rhabdoid tumor (ATRT); brain stem glioma; chordoma; chondrosarcoma; choroid plexus; CNS lymphoma; craniopharyngioma; cysts; ependymoma; ganglioglioma; germ cell tumor; glioblastoma (GBM); glioma; hemangioma; juvenile pilocytic astrocytoma (JPA); lipoma; lymphoma; medulloblastoma; meningioma; metastatic brain tumor; neurilemmomas; neurofibroma; neuronal & mixed neuronal-glial tumors; non-Hodgkin lymphoma; oligoastrocytoma; oligodendroglioma; optic nerve glioma; pineal tumor; pituitary tumor; primitive neuroectodermal (PNET); rhabdoid tumor; or schwannoma. An astrocytoma can be grade I pilocytic astrocytoma, grade II—low-grade astrocytoma, grade III anaplastic astrocytoma, grade IV glioblastoma (GBM), or a juvenile pilocytic astrocytoma. A glioma can be a brain stem glioma, ependymoma, mixed glioma, optic nerve glioma, or subependymoma.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| | | Bases | Complementary |
|---|---|---|---|
| Base | Name | Represented | Base |
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as an enhancer and/or promoter regions and lead to efficient transcription of the gene carried on the expression vector. The goal of a well-designed expression vector is the efficient production of protein, and this may be achieved by the production of a significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as: (i) by disabling repressors. The gene is expressed because an inducer binds to the repressor. The binding of the inducer to the repressor prevents the repressor from binding to the operator. RNA polymerase can then begin to transcribe operon genes, or (ii) by binding to activators. Activators generally bind poorly to activator DNA sequences unless an inducer is present. An activator binds to an inducer and the complex binds to the activation sequence and activates a target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as many as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A ribosomal skipping sequence (e.g., 2A sequence such as furin-GSG-T2A) can be used in a construct to prevent covalently linking translated amino acid sequences. In addition to T2A, F2A, P2A, or E2A can be used.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed”, “transgenic”, and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

“Point mutation” refers to when a single base pair is altered. A point mutation or substitution is a genetic mutation where a single nucleotide base is changed, inserted or deleted from a DNA or RNA sequence of an organism's genome. Point mutations have a variety of effects on the downstream protein product—consequences that are moderately predictable based upon the specifics of the mutation. These consequences can range from no effect (e.g., synonymous mutations) to deleterious effects (e.g., frameshift mutations), with regard to protein production, composition, and function. Point mutations can have one of three effects. First, the base substitution can be a silent mutation where the altered codon corresponds to the same amino acid. Second, the base substitution can be a missense mutation where the altered codon corresponds to a different amino acid. Or third, the base substitution can be a nonsense mutation where the altered codon corresponds to a stop signal. Silent mutations result in a new codon (a triplet nucleotide sequence in RNA) that codes for the same amino acid as the wild type codon in that position. In some silent mutations the codon codes for a different amino acid that happens to have the same properties as the amino acid produced by the wild type codon. Missense mutations involve substitutions that result in functionally different amino acids; these can lead to alteration or loss of protein function. Nonsense mutations, which are a severe type of base substitution, result in a stop codon in a position where there was not one before, which causes the premature termination of protein synthesis and can result in a complete loss of function in the finished protein.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively | K R H |
| Charged (Basic): | |
| Negatively | D E |
| Charged (Acidic): | |

Conservative Substitutions III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| Leu (L) | Ile, Val, Met, Ala, |
| | Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, |
| | Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, gene editing was used to disrupt expression of the cell's endogenous TCR. Processes for genome editing are well known, see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate blockage of endogenous TCR by genome editing can result in enhanced T cell function. Beyond enhancing the efficiency of receptor pairing, this also enables an allogeneic cell therapy product, a highly-sought goal in the development of cell-based immunotherapies.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for CART cell engineering by the removal of endogenous TCR expression.

### T Cell Engineering

As described herein, lentiviral-based T cell engineering to express the MHC-independent TCRs (miTCRs) combined with CRISPR-based gene editing was used to disrupt expression of the cell's endogenous TCR.

Any vector known in the art can be used. For example, the vector can be a viral vector selected from retrovirus, lentivirus, herpes, adenovirus, adeno-associated virus (AAV), rabies, Ebola, lentivirus, or hybrids thereof. As another example, non-viral vectors can be used including plasmid DNA (pDNA) or RNAi.

Genetic modification can be performed either ex vivo or in vivo. The ex vivo strategy is based on the modification of cells in culture and transplantation of the modified cell into a patient.

The use of endonucleases for targeted genome editing can utilize these enzymes as custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing cancer or tumor progression in a subject in need of administration of a therapeutically effective amount of a cell therapy comprising cells with miTCR constructs, so as to substantially inhibit cancer growth or proliferation, slow the progress of cancer growth or proliferation, or limit the development of cancer.

Methods described herein are generally performed on a subject in need thereof. A subject in need of the therapeutic methods described herein can be a subject having, diagnosed with, suspected of having, or at risk for developing cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a cell therapy is, for example, an amount that would cause the desired therapeutic effect in a subject while minimizing undesired side effects. In various embodiments, an effective amount of a cell therapy described herein can substantially inhibit cancer growth or proliferation, slow the progress of cancer growth or proliferation, or limit the development of cancer.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a cell therapy can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to substantially inhibit cancer growth or proliferation, slow the progress of cancer growth or proliferation, or limit the development of cancer.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a cell therapy can occur as a single event or over a time course of treatment. For example, a cell therapy can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for cancer treatment.

### Immunotherapy

As described herein, the provided compositions and methods allow for the treatment of cancer with a miTCR expressing cell. Immunotherapies are a new generation of cancer therapy that has revolutionized the treatment of otherwise terminal cancers, often achieving durable, sustained remission in cancers that were otherwise thought to be refractory to standard first- and second-line therapies. Thousands of patients annually are now treated with these life-saving therapies.

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) or serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Examples of immunotherapy can be immune effector cell (IEC) therapy (e.g., miTCR T cells) or T cell engaging therapy (e.g., CD19-specific T cell engager, such as blinatumomab, T cell engaging monoclonal antibody, bispecific T cell engager (BiTE) therapy).

In some embodiments, the provided methods are used before, after, or in concurrence with any form of BsMAb therapy. For example, the BsMAb therapy can be any one or more of the currently FDA-approved BsMAb therapies, such as blinatumomab, emicizumab, or amivantamab.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the T cells to the cancer target.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides, et al., 1998), cytokine therapy, e.g., interferons α, β, and γ; IL-1, GM-CSF, TNF (Bukowski, et al., 1998; Davidson, et al., 1998; Hellstrand, et al., 1998) gene therapy, e.g., TNF, IL-1, IL-2, p53 (Qin et al., 1998; Austin-Ward and Villaseca, 1998; U.S. Pat. Nos. 5,830,880 and 5,846,945), and monoclonal antibodies, e.g., anti-ganglioside GM2, anti-HER-2, anti-p185 (Pietras, et al., 1998; Hanibuchi, et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton, et al., 1992; Mitchell, et al., 1990; Mitchell, et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg, et al., 1988; 1989).

In some embodiments, the immunotherapy in accordance with the present disclosure is miTCR T cell therapy (e.g., CD19-specific chimeric TCR). Generally, CAR T cell therapy refers to any type of immunotherapy in which a subject's T cells are genetically modified to express chimeric antigen receptors. These chimeric antigen receptors allow the T cells to more effectively recognize and subsequently destroy cancer cells. Typically, T cells are first harvested from a subject, genetically altered to express a CAR targeting an antigen of interest (e.g., an antigen expressed on the surface of a tumor or cancer cell), and then infused back into the subject. Once infused into the subject, CAR T cells bind to the target antigen and are activated, allowing them to proliferate and become cytotoxic.

### Checkpoint Immunotherapy

The constructs and methods described herein can be used in combination with checkpoint immunotherapy. An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response.

Cancer cells can find ways to use these checkpoints to avoid being attacked by the immune system. But drugs that target these checkpoints hold a lot of promise as a cancer treatment. These drugs are called checkpoint inhibitors. Checkpoint inhibitors used to treat cancer don't work directly on the tumor at all. They only take the brakes off an immune response that has begun but has not yet been working at its full force.

Checkpoint immunotherapy has been extensively shown to unleash T cell effector functions to control tumors in many cancer patients. However, tumor cells can evade immunological elimination by recruiting myeloid cells that induce an immunosuppressive state. Recent high dimensional profiling studies have shown that tumor-infiltrating myeloid cells are considerably heterogeneous, and may include both immunostimulatory and immunosuppressive subsets, although they do not fit the M1/M2 paradigm. Thus, depletion of suppressive myeloid cells from tumors, blockade of their functions, or induction of myeloid cells with immunostimulatory properties may provide important approaches for improving immunotherapy strategies, perhaps in synergy with checkpoint blockade.

Any immune checkpoint inhibitor known in the art can be used. For example, a PD-1 inhibitor can be used. These drugs are typically administered IV (intravenously). PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it tells the T cell to leave the other cell alone. Some cancer cells have large amounts of PD-L1, which helps them hide from an immune attack.

Monoclonal antibodies that target either PD-1 or PD-L1 can block this binding and boost the immune response against cancer cells. These drugs have shown a great deal of promise in treating certain cancers.

Examples of drugs that target PD-1 can include Pembrolizumab (Keytruda), Nivolumab (Opdivo), or Cemiplimab (Libtayo). These drugs have been shown to be helpful in treating several types of cancer, and new cancer types are being added as more studies show these drugs to be effective.

As another example, a PD-L1 inhibitor can be used. Examples of drugs that target PD-L1 can include Atezolizumab (Tecentriq), Avelumab (Bavencio), or Durvalumab (Imfinzi). These drugs have also been shown to be helpful in treating different types of cancer, and are being studied for use against others.

CTLA-4 is another protein on some T cells that acts as a type of “off switch” to keep the immune system in check. For example, Ipilimumab (Yervoy) is a monoclonal antibody that attaches to CTLA-4 and reduces or blocks its function. This can boost the body's immune response against cancer cells. This drug can be used to treat melanoma of the skin and other cancers.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation have gained significant interest by researchers as a potential new therapeutic strategy for a wide range of diseases, in particular for degenerative and immunogenic pathologies.

Allogeneic cell therapy or allogeneic transplantation uses donor cells from a different subject than the recipient of the cells. A benefit of an allogeneic strategy is that unmatched allogeneic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to antigen binding fragments, miTCR components, constructs, pairs, and plasmids. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit, instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: MHC-Independent TCRs (miTCRs) for Cancer Immunotherapy

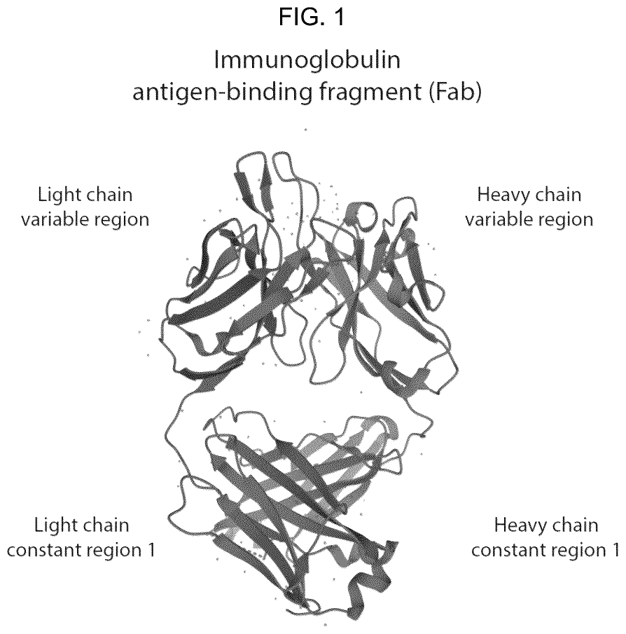

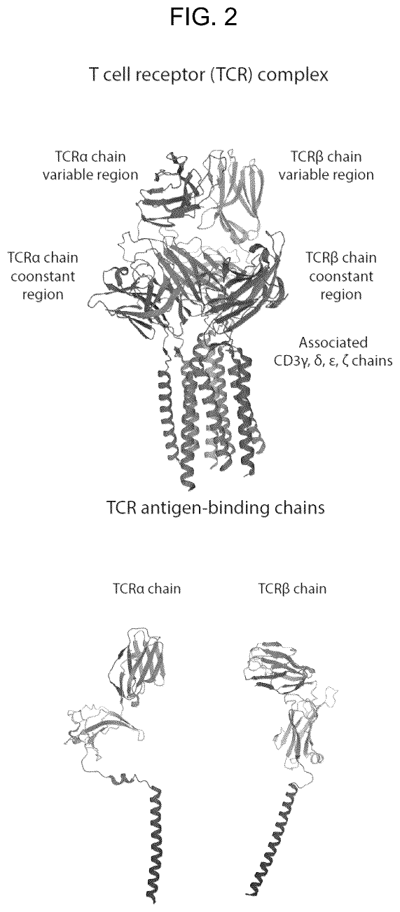

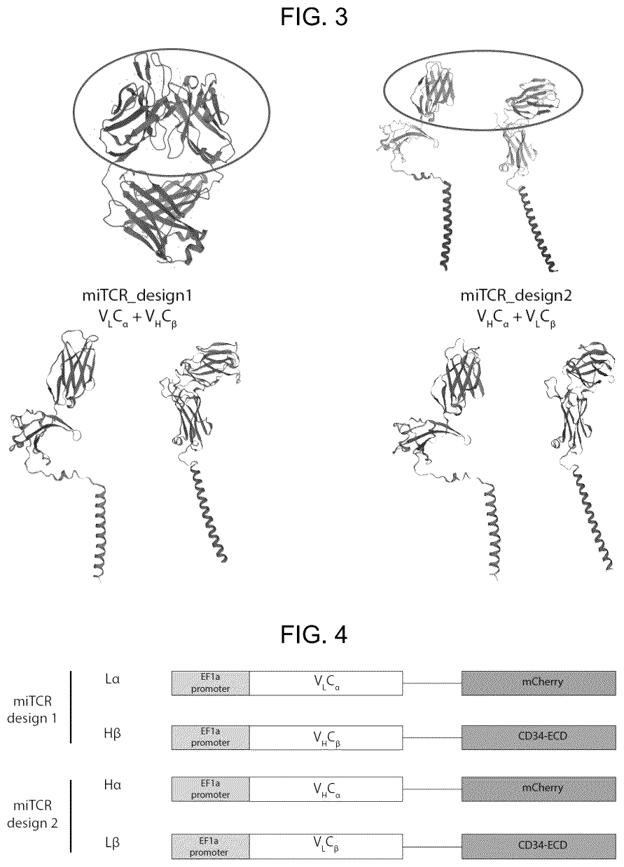

As described herein, an MHC-independent T cell receptor has been developed. The crystal structure of the immunoglobulin antigen-binding fragment (Fab) and TCR complex are shown in FIG. 1 and FIG. 2, respectively. By replacing the antigen-recognition domains of the TCR (alpha and beta variable chains) with the antigen-recognition domains of the immunoglobulin molecule (variable heavy (V_{H}) and light chains (V_{L})) (see e.g., FIG. 3), the miTCR was able to preserve the highly-regulated TCR complex-driven cellular activation and integrate MHC-independent antigen recognition.

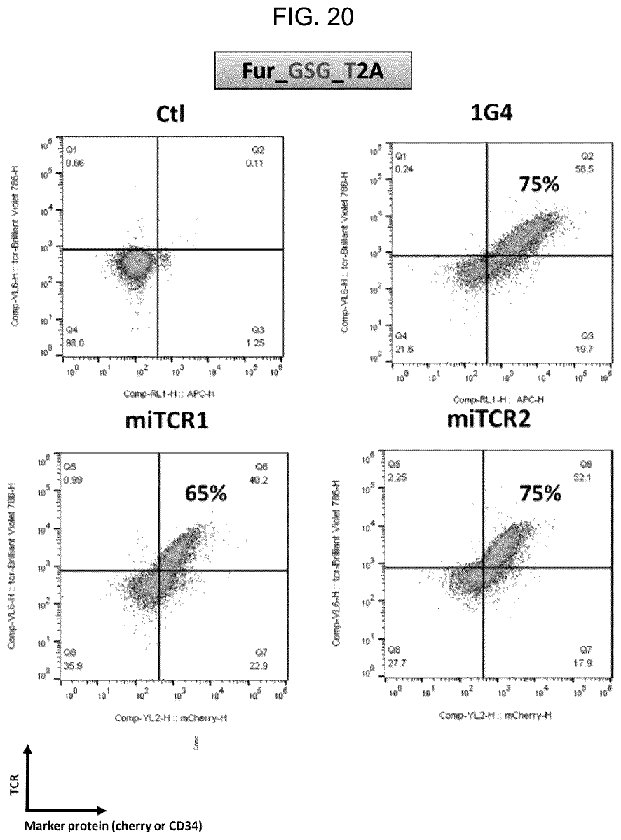

To implement this technology in an effective way, lentiviral-based T cell engineering was used to express the MHC-independent TCRs (miTCRs) combined with CRISPR-based gene editing to disrupt expression of the cell's endogenous TCR. >98% gene editing efficiency was achieved (see e.g., FIG. 21) in human primary T cells, which is believed to not have been previously accomplished, ensuring that nearly all translated alpha and beta TCR chains pair appropriately. Beyond enhancing efficiency of receptor pairing, this also enables an allogeneic cell therapy product, a highly-sought goal in the development of cell-based immunotherapies.

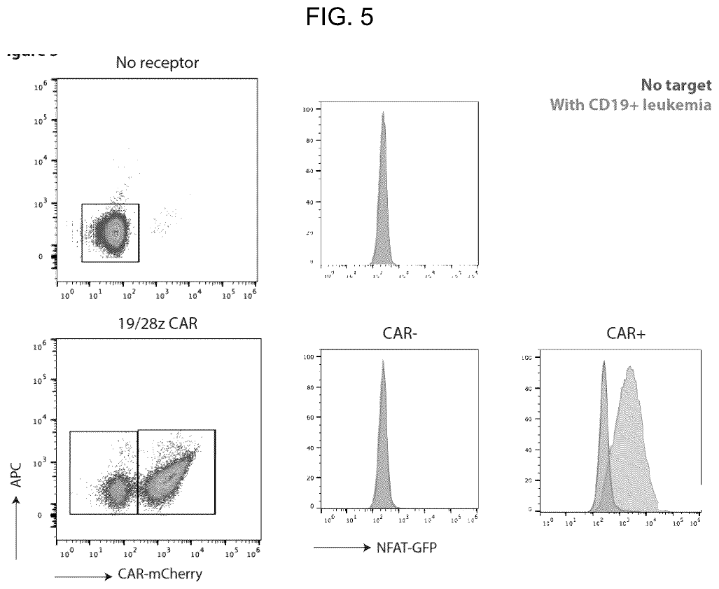

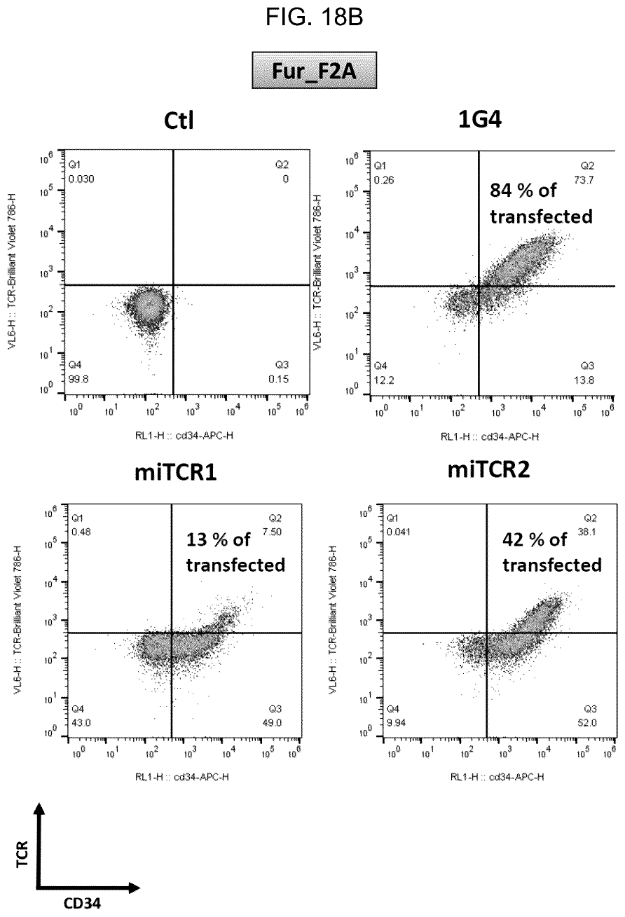

Four plasmid sequences were developed and are directed against the CD19 surface molecule (see e.g., FIG. 4). These mutants were expressed using the single-vector system (see e.g., FIG. 18A). They employ the FMC63 anti-CD19 antigen-recognition domains. They are paired variable heavy or variable light chains with TCR alpha or beta constant regions to create a single chain antigen receptor. Each alpha chain pairs with a beta chain to generate a complete TCR, which then self-assembles with the associated endogenous CD3 chains during intracellular packaging. Each plasmid also contains a selection marker (either mCherry or the extracellular domain of CD34) separated from the miTCR chain by a P2A skip sequence. The four constructs are (1) FMC63-vL_TCRalpha constant region_P2A_mCherry, (2) FMC63-vH_TCRalpha constant region_P2A_mCherry, (3) FMC63-vL_TCRbeta constant region_P2A_CD34EC domain, and (4) FMC63-vH_TCRbeta constant region_P2A_CD34EC domain.

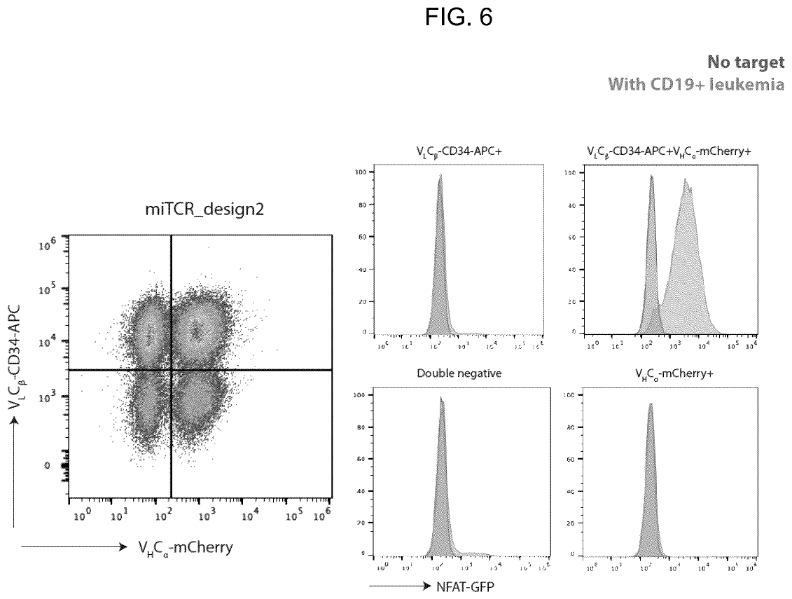

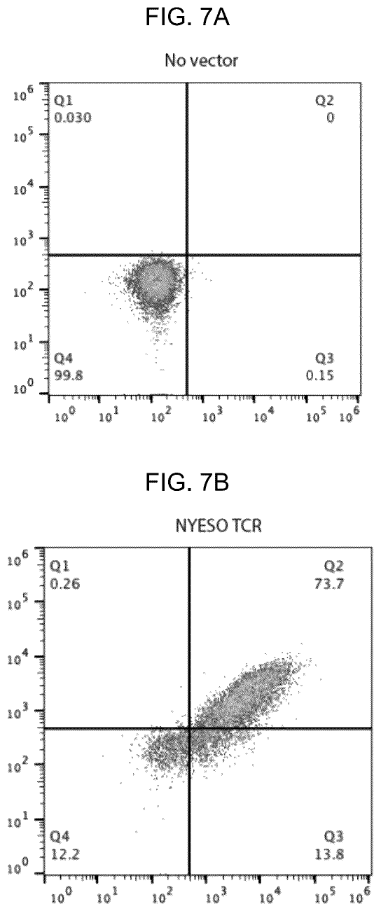

A Jurkat cell line containing an NFAT-GFP reporter was evaluated with flow cytometry after 18 hour co-incubation with either no target or with CD19+ leukemia (see e.g., FIG. 5). The Jurkat cells expressed the miTCR chains and demonstrated potent NFAT activation only with expression of both the beta and alpha chain (see e.g., FIG. 6).

### Example 2: Mutations to Increase Expression and Function

This example describes discovery that the variable chain/constant chain interface influences receptor expression.

Because it was discovered that the construct described in Example 1 had some issues being expressed on T cells, molecular modeling and the native TCR were compared, and a series of mutations were generated that may improve expression and function. These mutations were characterized and then compared to conventional CAR-Ts.

The basis for this observation is the difference in surface expression observed for miTCR1 (V_{L}-C_{α}+V_{H}-C_{β}) and miTCR2 (V_{H}-C_{α}+V_{L}-C_{β}), in which miTCR2 is consistently expressed at higher levels. Given that the only difference between these receptors is the orientation of the variable region and constant region interface, this led to the hypothesis that the interaction between these regions influenced surface expression.

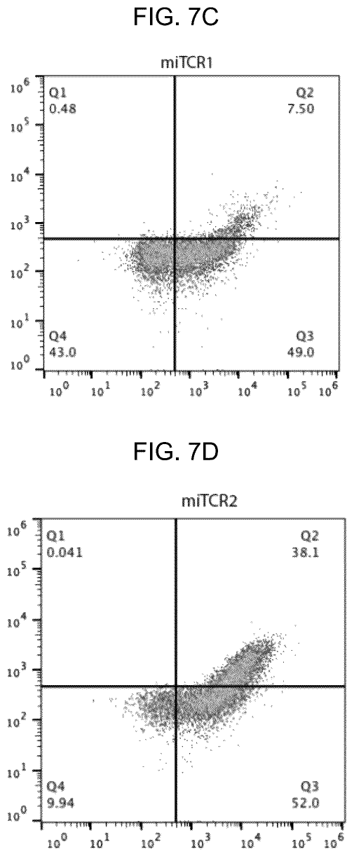

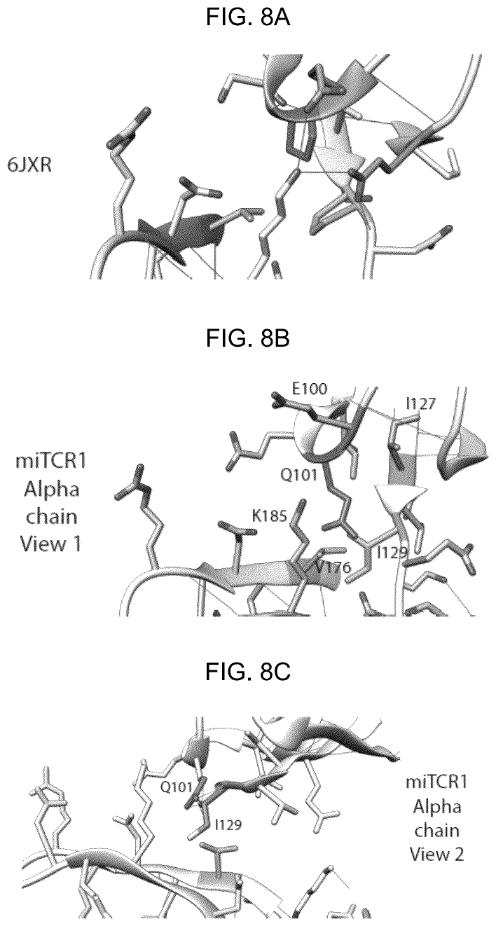

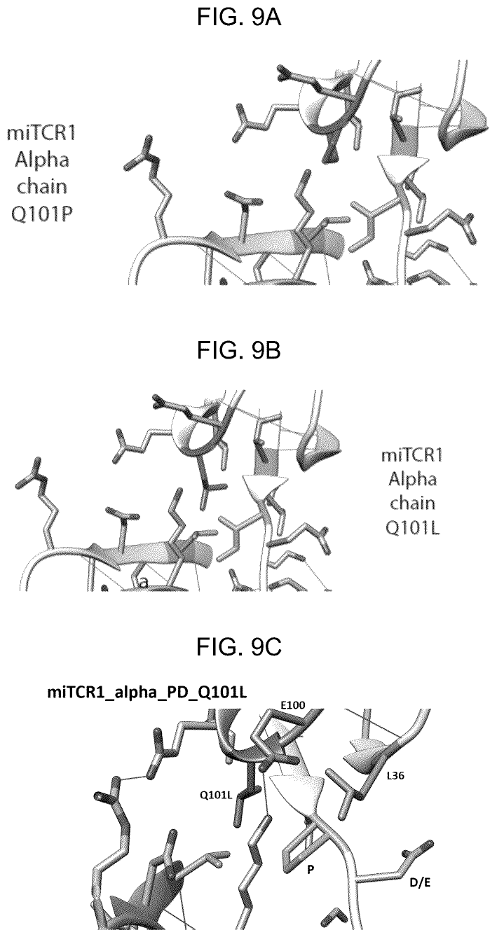

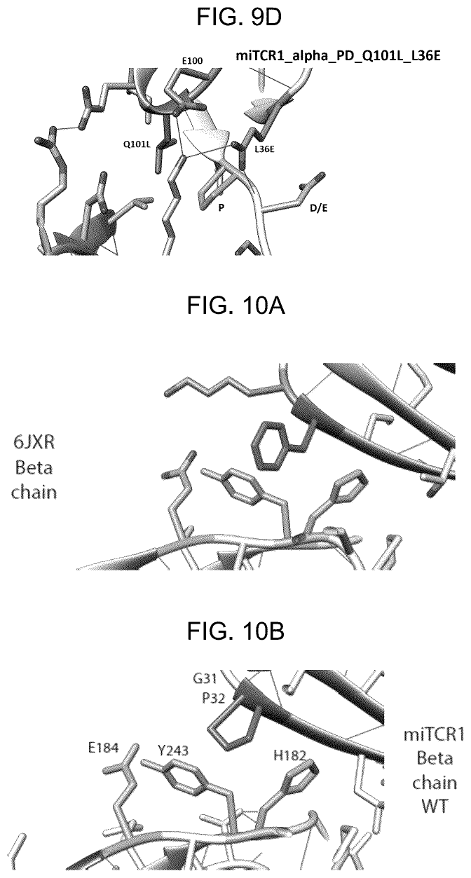

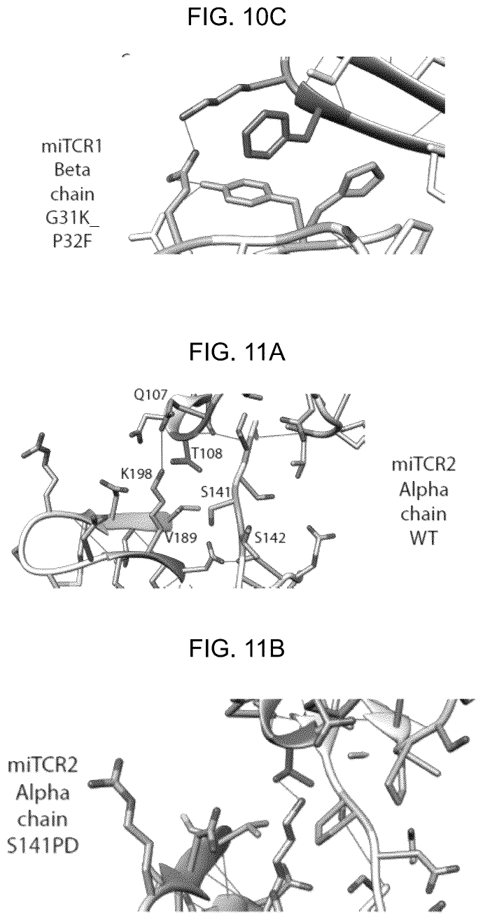

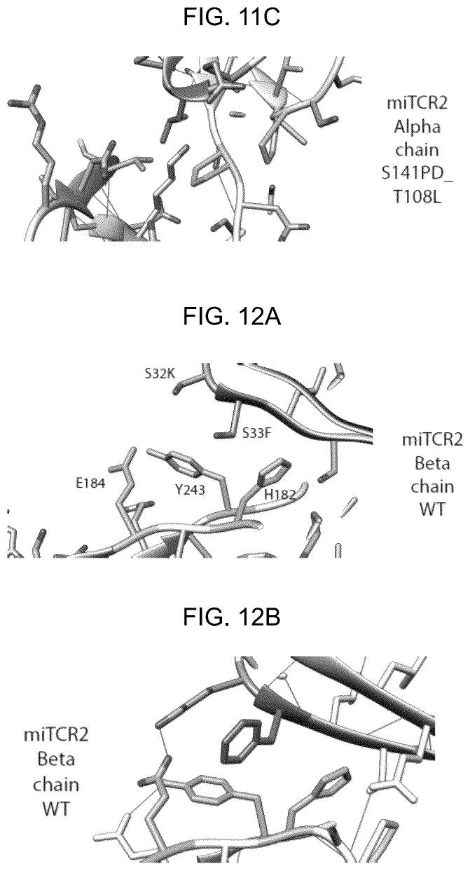

As demonstrated in FIG. 7A-FIG. 7D, endogenous TCRs that are composed of native TCR variable and constant regions express well, while miTCR1 expresses poorly and miTCR2 is intermediate. Predictive protein modeling highlighted that the miTCR1 alpha chain has many amino acid conflicts (see e.g., FIG. 8A-FIG. 8C) that, based on published data would impair surface receptor expression. Similar protein modeling revealed fewer conflicts in the miTCR1 beta (see e.g., FIG. 10A-FIG. 10B), miTCR2 alpha (see e.g., FIG. 11A), and miTCR2 beta chains (see e.g., FIG. 12A). Using rational amino acid mutations, several iterations of all four chains were designed (see e.g., FIG. 9A-FIG. 9D, FIG. 10C, FIG. 11B-FIG. 11C, and FIG. 12B) for evaluation to see how these mutations impact surface expression and therein receptor-driven T cell function.

### Example 3: Optimization and Demonstration of miTCR Function

This example describes the use of specific genetic constructs and amino acid substitutions to optimize the expression of miTCRs on the cell surface.

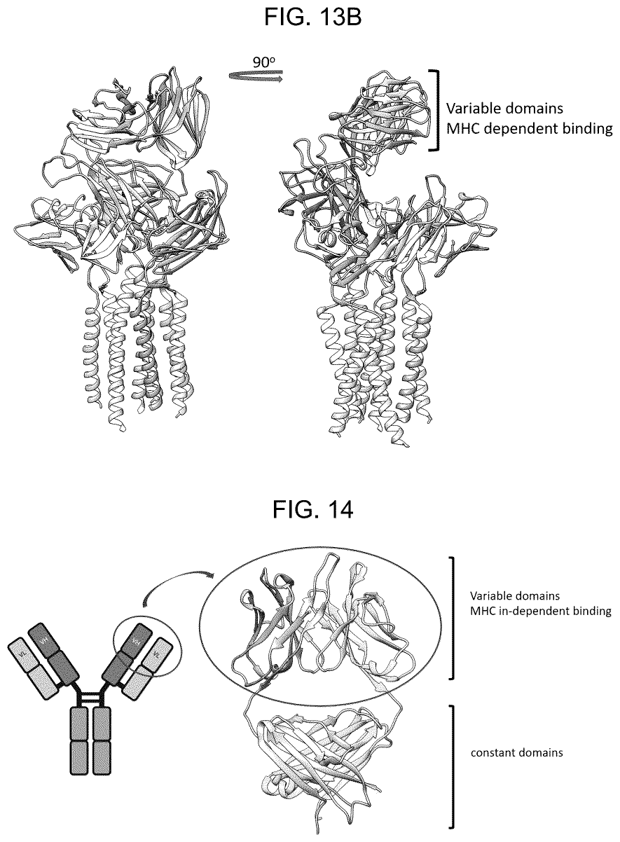

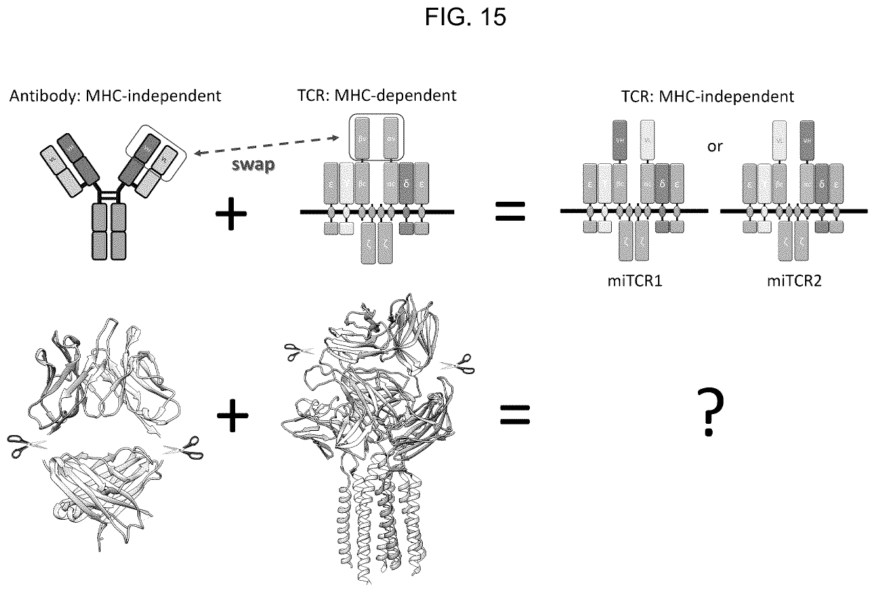

FIG. 13 describes a TCR structure (see e.g., Nature 573, pages 546-552 (2019)). FIG. 14 describes an antibody structure (see e.g., Proteins (2018) 86 p. 495-500). FIG. 15 describes the combination of the TCR constant regions and antibody variable regions to form an MHC-independent TCR.

The structure of the endogenous T cell receptor with associated CD3 molecules is shown in FIG. 13A-FIG. 13B. This receptor is dependent on MHC presentation to recognize a target antigen. The structure of the endogenous B cell receptor (immunoglobulin, BCR) is shown in FIG. 14. This receptor is independent of MHC for antigen recognition.

The general design scheme of MHC-independent TCRs (miTCR) is shown in FIG. 15. The antigen-binding region of the TCR is replaced with that from the BCR.

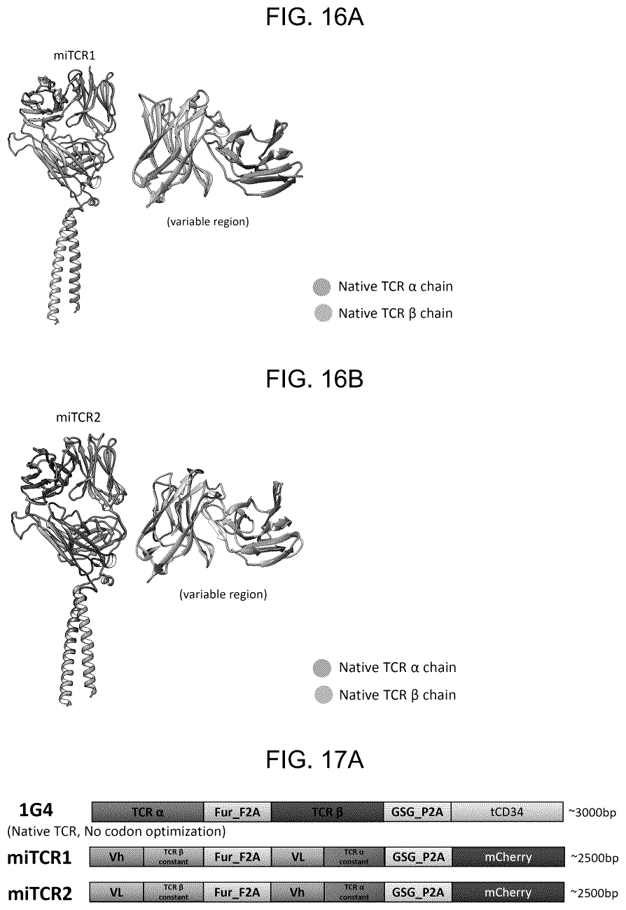

Predicted miTCR structures have similar protein folding as native TCR on a whole. FIG. 16A-FIG. 16B shows the predicted structure of the miTCRs. miTCR1 has the TCR alpha constant chain linked to the BCR variable light chain and the TCR beta constant chain linked to the BCR variable heavy chain (see e.g., FIG. 16A). miTCR2 has the TCR alpha constant chain linked to the BCR variable heavy chain and the TCR beta constant chain linked to the BCR variable light chain (see e.g., FIG. 16B). Here, CRISPR was used to remove endogenous TCR alpha and beta chains to prevent mispairing with the chimeric miTCR chains in a cell. The TCR constant regions of the miTCR chains have been mutated (at the DNA level, not protein level) to be resistant to the CRISPR engineering.

These are the DNA sequences in all of our DNA to avoid being CRISPRed out. The mutations are enlarged and bolded:

TCR constant alpha region resistant to CRISPR
(SEQ ID NO: 45):
CGTGCTGGATATGCGCAGCA
TCR constant beta region resistant to CRISPR
(SEQ ID NO: 46):
CGAAAAcGATGAaTGGACaC

These DNA changes for resistance to the CRE do not change the protein sequence.

Guide RNA for knocking out endogenous TCR:

(SEQ ID NO: 47)
TGTGCTAGACATGAGGTCTA = TRAC
(SEQ ID NO: 48)
GGAGAATGACGAGTGGACCC = TRBC

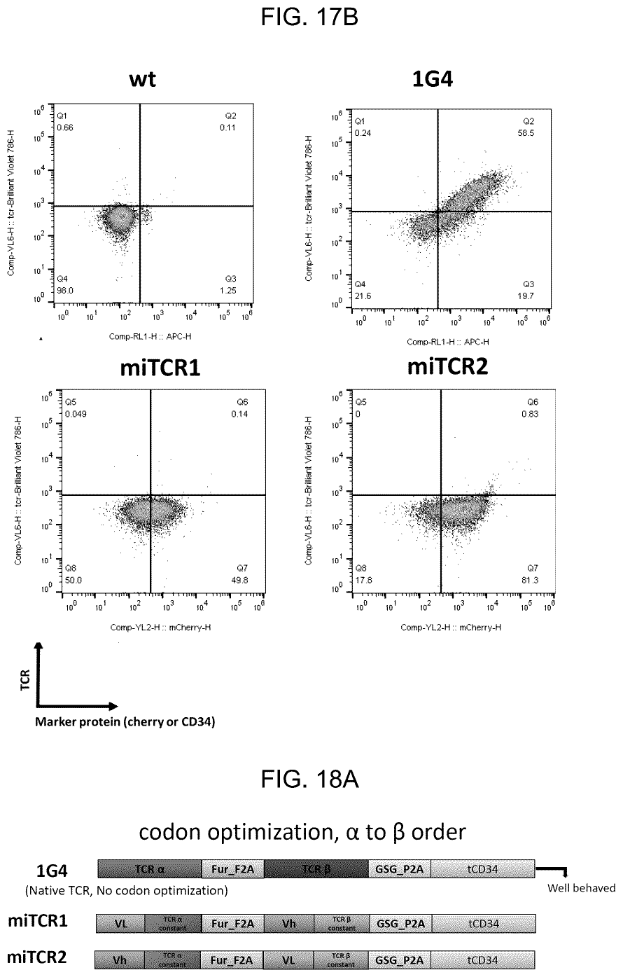

Compared to a full-length native TCR (1G4), the miTCRs did not express as well on the cell surface. The transgenic miTCR DNA sequences have an F2A ribosomal skip sequence between the alpha and beta chains, followed by a P2A skip sequence that separates a third transgene, which is a reporter construct (see e.g., FIG. 17A). FIG. 17A shows miTCRs with no-codon optimization in β to α order. Flow cytometry images show that the correlation between the expression of the reporter and the native TCR is very high, while this is not the case for miTCRs (see e.g., FIG. 17B). These experiments were performed in a Jurkat TCR knockout cell line.

By optimizing codon sequences for expression in human cells (see e.g., FIG. 18A, in α to β order), the surface expression of miTCRs is improved (see e.g., FIG. 18B). However, surface expression is still significantly different between 1G4 and miTCRs (see e.g., FIG. 18B). These experiments were performed in a Jurkat TCR knockout cell line.

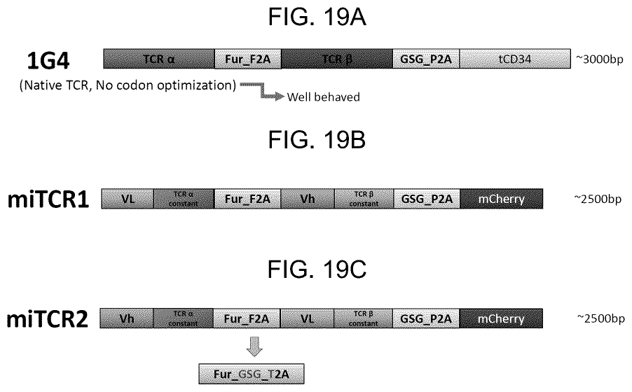

It was hypothesized that this results from the efficiency of the F2A sequence in a DNA sequence-dependent manner, or simply from stability of the chimeric miTCRs on the cell surface (FIG. 19D). Therefore, the F2A site was changed to a GSG-T2A site, which is thought to be more efficient in human cells (see e.g., FIG. 19A-FIG. 19C).

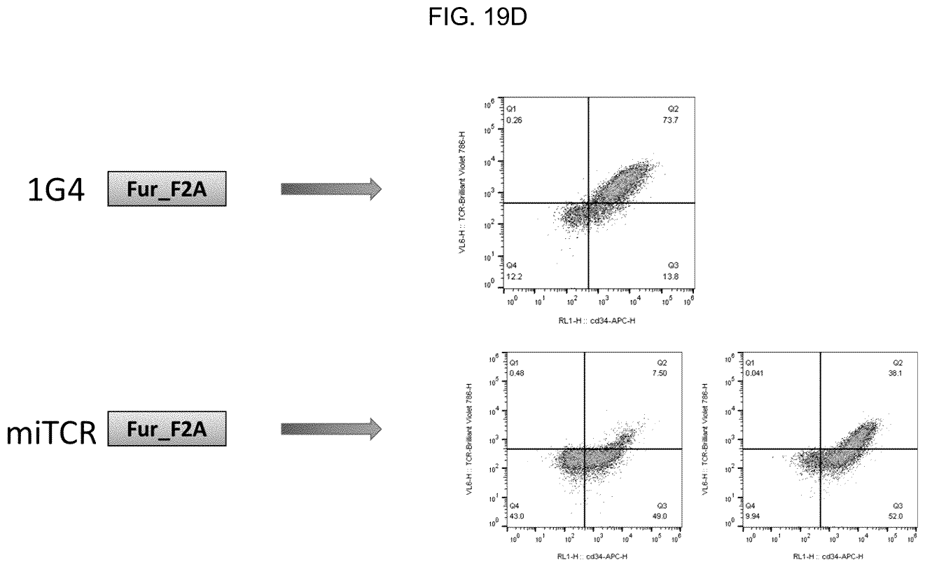

The optimized plasmid DNA containing the furin-GSG-T2A site was delivered to Jurkat cells (cell line) by transfection. This change from F2A to GSG-T2A significantly improved the surface expression of miTCRs (see e.g., FIG. 20 compared to FIG. 18B), suggesting that the specific ribosomal skip sequence influences the expression of these chimeric receptors.

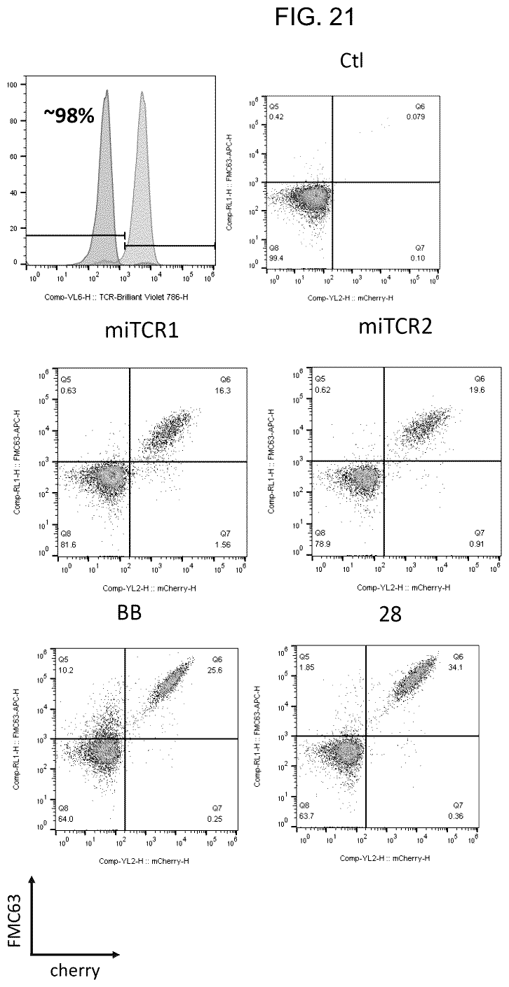

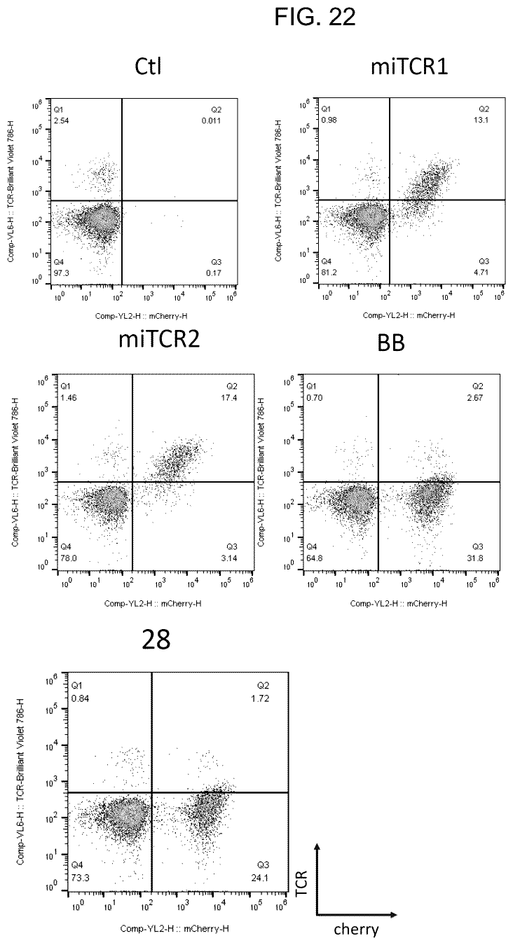

The lentivirus produced using these plasmids, efficiently expressed miTCRs on the cell surface in primary human T cells (see e.g., FIG. 21). miTCRs were detected using an antibody against FMC63, the parental BCR used to manufacture the anti-CD19 variable chains. Endogenous TCR was knocked out to prevent or reduce mispairing. Similarly, miTCRs were also detected on the surface of human T cells using an antibody directed against the TCR constant chains (see e.g., FIG. 22). Further, cells that express TCR constant chains also stained with anti-FMC63, demonstrating that both regions of this chimeric protein (variable from BCR and constant from TCR) can be detected on the same cells (see e.g., FIG. 23).

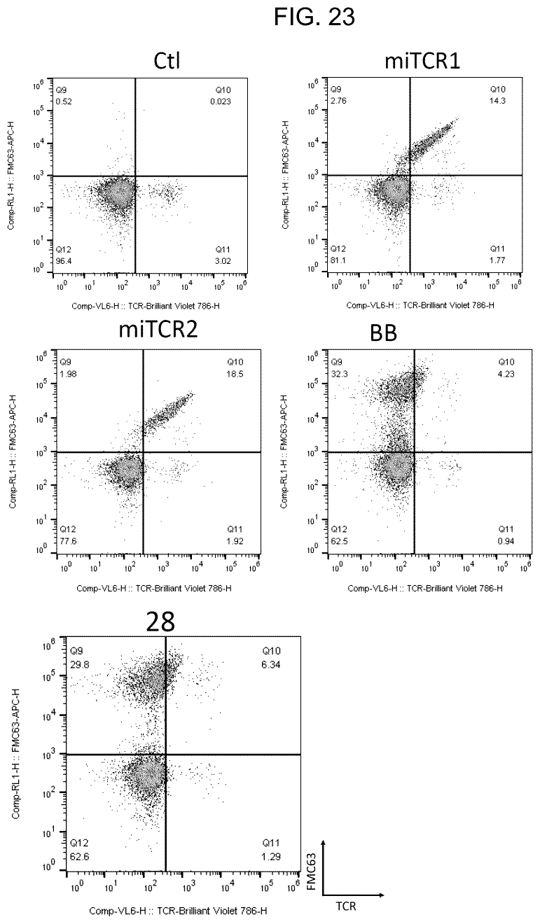

It was also shown that the expression of miTCRs results in TCR complex assembly. CD3 epsilon, a key component of the full TCR complex (see e.g., FIG. 13A-FIG. 13B) requires the TCR alpha and beta chains for surface expression—this complex can only be expressed on the cell surface if all components are present. As a control, CRISPR was used to remove the alpha and beta chains but no constant chain-containing constructs were added, resulting in little CD3 surface expression (see e.g., FIG. 24, top left). The addition of miTCRs significantly increased surface CD3, suggesting that miTCRs form native TCR-like complexes (see e.g., FIG. 24, top right and middle left).

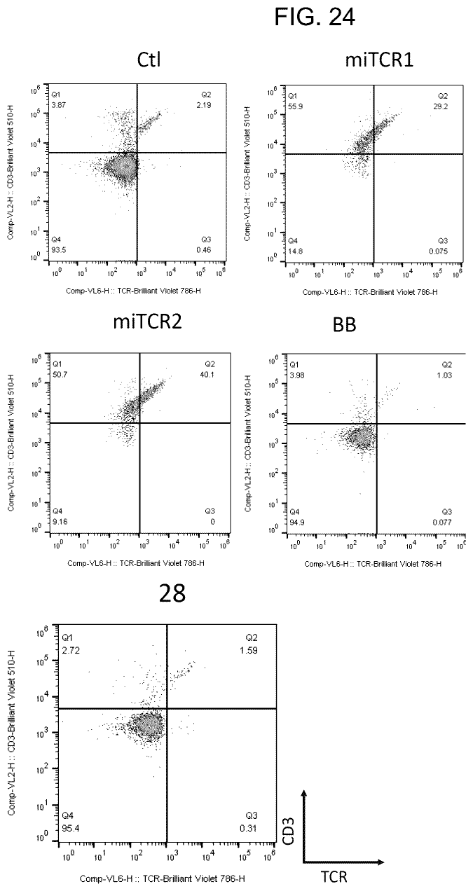

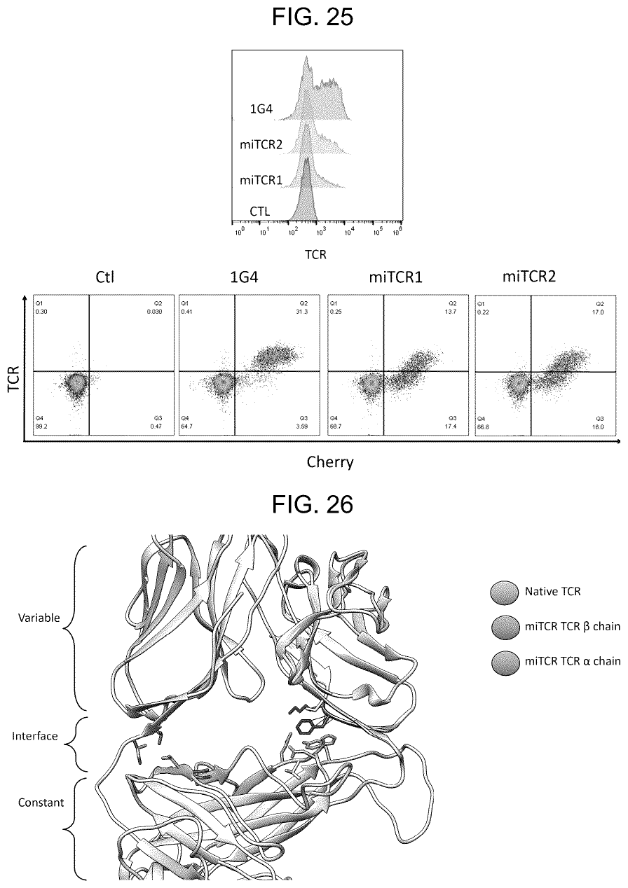

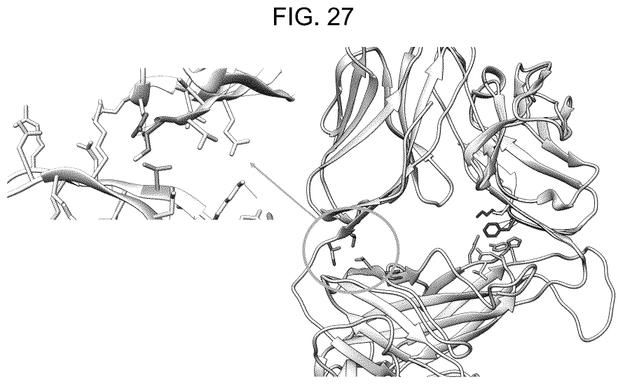

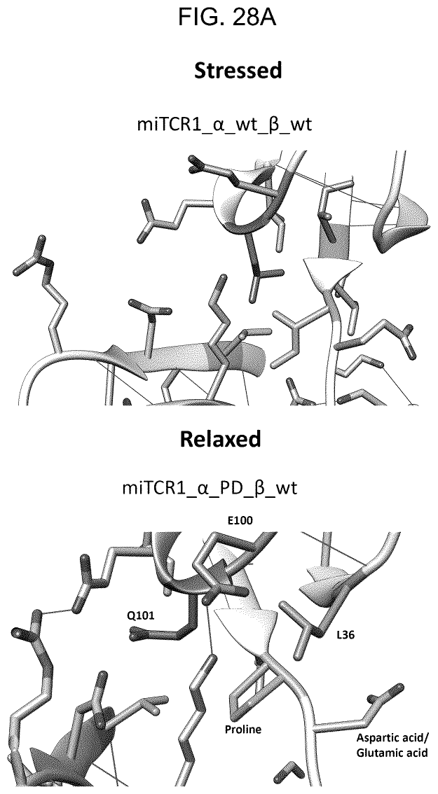

While these modifications improved miTCR expression, the miTCRs were still not expressed on the surface as efficiently as native TCRs (see e.g., FIG. 25). It was speculated that this is a result of protein hybridization—cutting and pasting two seemingly similar structures together. Since the structures of the variable and constant regions are not hybrids themselves, the interface between these two regions (variable and constant region protein-protein interface, which is important for stability) was focused on, since this is the area that has been modified from native structure (see e.g., FIG. 26). In this chimera protein, the native amino acid sequences in the variable and constant domains likely endow each domain to form proper protein folding. Any imperfect amino acid interactions are likely to be on the interface between the variable and constant domains, because the interface is constructed by two protein domains that do not associate in nature. Structural evaluation revealed that at this interface there are many chemical “conflicts”—interactions between amino acids that are not favorable and are likely to impact protein structure/folding. FIG. 8C, FIG. 27, and FIG. 28A provide more detailed illustrations of the clashes observed—primarily disruption of hydrophobic pockets that results from hybridization of variable BCR chains and constant TCR chains.

For example, a clash was found in miTCR1 alpha chain variable and constant interface (see e.g., FIG. 27, green circled region). It was observed that disruption of hydrophobic pockets resulted from the hybridization of variable BCR chains and constant TCR chains (see e.g., FIG. 8A-FIG. 8B and FIG. 28A-FIG. 28B). In native TCR, the proline, valine from the alpha constant chain (shown in orange) and another proline or hydrophobic residue from alpha variable chain (shown in purple) form a stable hydrophobic core in the interface. The additional hydrogen bonds in the region (shown as red line) strengthen the protein structure stability as a whole (see e.g., FIG. 8A). In the corresponding region of miTCR1, a polar residue Q101 (purple) from the antibody variable domain sticks into or extends into the hydrophobic core of the TCR constant region, disturbing stable hydrophobic interactions and disrupting hydrogen bond formation (see e.g., FIG. 8B).

Targeted modification of this region, for example by insertion of an additional proline and aspartic acid, resolves this conflict by recreating the hydrophobic pocket that is present in the native TCR structure (see e.g., FIG. 8A and FIG. 28). After inserting proline and aspartic acid (or glutamic acid) (PD or PE or PY) in the junction in the TCR alpha chain constant region (e.g., miTCR1_α_PD_β_wt), the miTCR1 mutant was predicted to adopt a more relaxed status in the interface, and regain or restore the hydrogen bonding for more protein stability (see e.g., FIG. 28A).

Based on this, a detailed analysis was performed of all amino acid interactions at the variable chain/constant chain interface for both miTCR1 and miTCR2. An example of further modifications that were instituted—mutation of Q101 to L, L36 to E, in addition to the insertion of the PD is shown in FIG. 9B-FIG. 9D and FIG. 28B. This process led to the development of 14 additional constructs with targeted changes at sites of potential conflict (see e.g., TABLE 1 and TABLE 2). Sequences of the constructs are provided in TABLE 3 and TABLE 4. Of note, changes were made to specific amino acids (e.g., G31>K), but any changes using amino acids with similar chemical properties could be used (e.g., G31>R or Q). For example, residues with similar chemical properties that can be interchangeable are: (i) hydrophobic side chains: L, I, M, V; (ii) electrically charged side chains: R, K, Q; (iii) aromatic side chains: Y, F, H, W.

TABLE 1
miTCR1 constructs with amino acid modifications in
the alpha or beta chains, as indicated by genotype.

| Construct name | Genotype |
|---|---|
| JFC029 | miTCR1_α_wt_β_wt |
| JFC030 | miTCR1_α_wt_β_G31K_P32F |
| JFC031 | miTCR1_α_PD_β_wt |
| JFC032 | miTCR1_α_PD_β_G31K_P32F |
| JFC033 | miTCR1_α_Q101L_β_wt |
| JFC034 | miTCR1_α_PD_Q101L_β_wt |
| JFC035 | miTCR1_α_PD_Q101L_L36E_β_wt |
| JFC036 | miTCR1_α_Q101L_β_G31K_P32F |
| JFC037 | miTCR1_α_PD_Q101L_β_G31K_P32F |
| JFC038 | miTCR1_α_PD_Q101L_L36E_β_G31K_P32F |

TABLE 2
miTCR2 constructs with amino acid modifications in
the alpha or beta chains, as indicated by genotype.

| Construct name | Genotype |
|---|---|
| JFC039 | miTCR2_α_wt_ß_wt |
| JFC040 | miTCR2_α_wt_β_S32K_S33F |
| JFC041 | miTCR2_α_S141PD_β_wt |
| JFC042 | miTCR2_α_S141PD_β_S32K_S33F |
| JFC043 | miTCR2_α_S141PD_T108L_β_wt |
| JFC044 | miTCR2_α_S141PD_T108L_β_S32K_S33F |

TABLE 3
DNA (nucleotide) sequences of miTCR1 and miTCR2
constructs. Modifications to the parent constructs are depicted
by bolded and double-underlined text.

JFC029: miTCR1_α_wt_β_wt (SEQ ID NO: 1)
ATGGAGACCCTGCTGGGCCTGCTGATCCTGTGGCTGCAGCTGCAGTGGGTGAG
CTCCAAGCAGGACATCCAGATGACCCAGACCACATCTAGCCTGTCTGCCAGCCT
GGGCGATAGGGTGACAATCTCCTGCCGCGCCTCTCAGGACATCAGCAAGTATCT
GAACTGGTACCAGCAGAAGCCAGATGGCACCGTGAAGCTGCTGATCTATCACAC
ATCCCGGCTGCACTCTGGCGTGCCCAGCAGATTTTCCGGATCTGGAAGCGGAA
CCGACTACTCCCTGACAATCTCTAACCTGGAGCAGGAGGATATCGCCACCTATT
TTTGTCAGCAGGGCAATACCCTGCCTTACACATTCGGCGGCGGCACCAAGCTGG
AGATCACAATCCAGAACCCTGACCCAGCCGTGTATCAGCTGAGGGACTCCAAGT
CCTCTGATAAGAGCGTGTGCCTGTTCACCGACTTTGATTCTCAGACAAACGTGA
GCCAGTCTAAGGACAGCGACGTGTACATCACCGACAAGTGCGTGCTGGATATGC
GCAGCATGGACTTCAAGAGCAACTCCGCCGTGGCCTGGAGCAATAAGTCCGATT
TCGCCTGCGCCAACGCCTTTAACAATTCCATCATCCCAGAGGATACATTCTTTCC
CTCTCCTGAGAGCTCCTGTGACGTGAAGCTGGTGGAGAAGAGCTTCGAGACCG
ATACAAACCTGAATTTTCAGAACCTGTCCGTGATCGGCTTCCGGATCCTGCTGCT
GAAGGTGGCCGGCTTTAATCTGCTGATGACCCTGAGACTGTGGTCTAGCAGGG
CCAAGCGCGGCAGCGGCGAGGGCAGAGGAAGTCTGCTGACATGCGGAGACGT
GGAGGAGAATCCTGGACCTATGTCCATCGGACTGCTGTGCTGTGCCGCCCTGA
GCCTGCTGTGGGCCGGCCCAGTGAACGCAGGAGTGGAGGTGAAGCTGCAGGA
GTCCGGCCCCGGACTGGTGGCACCTTCTCAGAGCCTGTCCGTGACCTGCACAG
TGTCTGGCGTGAGCCTGCCAGACTACGGCGTGAGCTGGATCAGGCAGCCACCT
AGAAAGGGACTGGAGTGGCTGGGCGTGATCTGGGGCTCCGAGACCACATACTA
TAATAGCGCCCTGAAGTCCCGGCTGACCATCATCAAGGATAACTCTAAGAGCCA
GGTGTTTCTGAAGATGAATAGCCTGCAGACCGACGATACAGCCATCTACTATTG
CGCCAAGCACTACTATTACGGCGGCTCTTATGCCATGGACTACTGGGGCCAGG
GCACCAGCGTGACAGTGTCCTCTGATCTGAAGAACGTGTTCCCACCCGAGGTG
GCCGTGTTTGAGCCTTCTGAGGCCGAGATCAGCCACACCCAGAAGGCCACCCT
GGTGTGCCTGGCAACCGGCTTCTATCCAGACCACGTGGAGCTGAGCTGGTGGG
TGAACGGCAAGGAGGTGCACTCCGGCGTGTGCACAGACCCTCAGCCACTGAAG
GAGCAGCCCGCCCTGAATGATTCCCGGTATTGTCTGAGCTCCCGGCTGAGAGT
GTCTGCCACCTTTTGGCAGAACCCTAGGAATCACTTCCGCTGCCAGGTGCAGTT
TTACGGCCTGAGCGAAAAcGATGAaTGGACaCAGGACAGGGCCAAGCCCGTGAC
ACAGATCGTGTCCGCCGAGGCATGGGGAAGGGCAGACTGTGGCTTCACCTCCG
AGTCTTACCAGCAGGGCGTGCTGAGCGCCACCATCCTGTATGAGATCCTGCTGG
GCAAGGCCACACTGTACGCCGTGCTGGTGTCTGCCCTGGTGCTGATGGCCATG
GTGAAGCGGAAGGATTCCAGAGGCCGGACCGATGGAAGCGGAGCTACTAACTT
CAGCCTGCTGAAGCAGGCTGGAGACGTGGAGGAGAACCCTGGACCTGTTTCTG
AAGCCATGGTGTCTAAGGGCGAGGAGGATAATATGGCCATCATCAAGGAGTTCA
TGAGGTTTAAGGTGCACATGGAGGGCAGCGTGAACGGCCACGAGTTTGAGATC
GAGGGAGAGGGAGAGGGCCGCCCATACGAGGGAACCCAGACAGCCAAGCTGA
AGGTGACCAAGGGCGGCCCTCTGCCTTTTGCCTGGGACATCCTGTCCCCTCAGT
TCATGTACGGCTCTAAGGCCTATGTGAAGCACCCAGCCGACATCCCCGATTACC
TGAAGCTGTCCTTCCCAGAGGGCTTTAAGTGGGAGAGAGTGATGAACTTCGAGG
ATGGCGGCGTGGTGACCGTGACACAGGATTCTAGCCTGCAGGACGGCGAGTTT
ATCTACAAGGTGAAGCTGAGAGGCACCAATTTCCCTAGCGACGGCCCAGTGATG
CAGAAGAAGACAATGGGCTGGGAGGCCTCCTCTGAGAGGATGTATCCTGAGGA
TGGCGCCCTGAAGGGCGAGATCAAGCAGCGCCTGAAGCTGAAGGATGGCGGC
CACTACGACGCCGAGGTGAAGACCACATATAAGGCCAAGAAGCCAGTGCAGCT
GCCTGGAGCCTACAACGTGAATATCAAGCTGGATATCACCAGCCACAATGAGGA
CTACACAATCGTGGAGCAGTATGAGAGGGCAGAGGGCAGACACTCCACCGGAG
GCATGGACGAGCTGTATAAGTAAGCGGCCGCCACCCAGCTTTCTTGTACAAAGT
GGT
JFC039: miTCR2_α_wt_β_wt (SEQ ID NO: 2)
ATGGAGACCCTGCTGGGCCTGCTGATCCTGTGGCTGCAGCTGCAGTGGGTGAG
CTCCAAGCAGGAGGTGAAGCTGCAGGAGAGCGGCCCCGGACTGGTGGCACCTT
CTCAGAGCCTGTCCGTGACCTGCACAGTGAGCGGCGTGTCCCTGCCTGACTAC
GGCGTGAGCTGGATCAGGCAGCCACCTAGAAAGGGACTGGAGTGGCTGGGCG
TGATCTGGGGCTCCGAGACCACATACTATAACTCCGCCCTGAAGTCTCGGCTGA
CCATCATCAAGGATAACTCTAAGAGCCAGGTGTTTCTGAAGATGAATTCCCTGCA
GACCGACGATACAGCCATCTACTATTGTGCCAAGCACTACTATTACGGCGGCTC
TTATGCCATGGACTACTGGGGCCAGGGCACCAGCGTGACAGTGTCTAGCATCCA
GAACCCTGATCCAGCCGTGTATCAGCTGAGGGACTCCAAGTCCTCTGATAAGAG
CGTGTGCCTGTTCACCGACTTTGATTCTCAGACAAACGTGAGCCAGTCTAAGGA
CAGCGACGTGTACATCACCGACAAGTGCGTGCTGGATATGCGCAGCATGGACTT
CAAGAGCAACTCCGCCGTGGCCTGGTCTAATAAGAGCGACTTCGCCTGCGCCA
ACGCCTTTAACAATTCCATCATCCCAGAGGATACATTCTTTCCCTCTCCTGAGAG
CTCCTGTGACGTGAAGCTGGTGGAGAAGAGCTTCGAGACCGATACAAACCTGAA
TTTTCAGAACCTGTCCGTGATCGGCTTCCGGATCCTGCTGCTGAAGGTGGCCGG
CTTTAATCTGCTGATGACCCTGAGACTGTGGTCTAGCAGGGCCAAGCGCGGCAG
CGGCGAGGGCAGAGGAAGTCTGCTGACATGCGGAGACGTGGAGGAGAATCCT
GGACCTATGTCCATCGGACTGCTGTGCTGTGCCGCCCTGAGCCTGCTGTGGGC
CGGCCCAGTGAACGCAGGCGTGGACATCCAGATGACCCAGACCACATCCTCTC
TGTCTGCCAGCCTGGGCGACAGGGTGACAATCAGCTGCCGCGCCTCCCAGGAT
ATCTCTAAGTATCTGAATTGGTACCAGCAGAAGCCAGATGGCACCGTGAAGCTG
CTGATCTATCACACAAGCCGGCTGCACTCCGGAGTGCCATCTAGATTTTCCGGA
TCTGGAAGCGGAACCGACTACAGCCTGACAATCTCCAACCTGGAGCAGGAGGA
TATCGCCACCTATTTTTGCCAGCAGGGCAATACCCTGCCCTACACATTCGGCGG
CGGCACCAAGCTGGAGATCACAGACCTGAAGAACGTGTTCCCACCCGAGGTGG
CCGTGTTTGAGCCTTCTGAGGCCGAGATCAGCCACACCCAGAAGGCCACCCTG
GTGTGCCTGGCAACCGGCTTCTATCCAGATCACGTGGAGCTGAGCTGGTGGGT
GAACGGCAAGGAGGTGCACTCCGGCGTGTGCACAGACCCTCAGCCACTGAAGG
AGCAGCCCGCCCTGAATGATTCCCGGTATTGTCTGAGCTCCCGGCTGAGAGTGT
CTGCCACCTTTTGGCAGAACCCTAGGAATCACTTCCGCTGCCAGGTGCAGTTTT
ACGGCCTGAGCGAAAACGATGAATGGACACAGGACAGGGCCAAGCCCGTGACA
CAGATCGTGTCCGCCGAGGCATGGGGAAGGGCAGACTGTGGCTTCACCTCCGA
GTCTTACCAGCAGGGCGTGCTGAGCGCCACCATCCTGTATGAGATCCTGCTGG
GCAAGGCCACACTGTACGCCGTGCTGGTGTCTGCCCTGGTGCTGATGGCCATG
GTGAAGCGGAAGGATTCCAGAGGCCGGACCGATGGAAGCGGAGCTACTAACTT
CAGCCTGCTGAAGCAGGCTGGAGACGTGGAGGAGAACCCTGGACCTGTTTCTG
AAGCCATGGTGTCTAAGGGCGAGGAGGATAATATGGCCATCATCAAGGAGTTCA
TGAGGTTTAAGGTGCACATGGAGGGCAGCGTGAACGGCCACGAGTTTGAGATC
GAGGGAGAGGGAGAGGGCCGCCCATACGAGGGAACCCAGACAGCCAAGCTGA
AGGTGACCAAGGGCGGCCCTCTGCCTTTTGCCTGGGACATCCTGTCCCCTCAGT
TCATGTACGGCTCTAAGGCCTATGTGAAGCACCCAGCCGACATCCCCGATTACC
TGAAGCTGTCCTTCCCAGAGGGCTTTAAGTGGGAGAGAGTGATGAACTTCGAGG
ATGGCGGCGTGGTGACCGTGACACAGGATTCTAGCCTGCAGGACGGCGAGTTT
ATCTACAAGGTGAAGCTGAGAGGCACCAATTTCCCTAGCGACGGCCCAGTGATG
CAGAAGAAGACAATGGGCTGGGAGGCCTCCTCTGAGAGGATGTATCCTGAGGA
TGGCGCCCTGAAGGGCGAGATCAAGCAGCGCCTGAAGCTGAAGGATGGCGGC
CACTACGACGCCGAGGTGAAGACCACATATAAGGCCAAGAAGCCAGTGCAGCT
GCCTGGAGCCTACAACGTGAATATCAAGCTGGATATCACCAGCCACAATGAGGA
CTACACAATCGTGGAGCAGTATGAGAGGGCAGAGGGCAGACACTCCACCGGAG
GCATGGACGAGCTGTATAAGTAAGC
JFC030: miTCR1_α_wt_β_G31K_P32F (SEQ ID NO: 3)
gtcgacgttaacgccgccaccATGGAGACCCTGCTGGGCCTGCTGATCCTGTGGCTGCAG
CTGCAGTGGGTGAGCTCCAAGCAGGACATCCAGATGACCCAGACCACATCTAG
CCTGTCTGCCAGCCTGGGCGATAGGGTGACAATCTCCTGCCGCGCCTCTCAGG
ACATCAGCAAGTATCTGAACTGGTACCAGCAGAAGCCAGATGGCACCGTGAAGC
TGCTGATCTATCACACATCCCGGCTGCACTCTGGCGTGCCCAGCAGATTTTCCG
GATCTGGAAGCGGAACCGACTACTCCCTGACAATCTCTAACCTGGAGCAGGAGG
ATATCGCCACCTATTTTTGTCAGCAGGGCAATACCCTGCCTTACACATTCGGCGG
CGGCACCAAGCTGGAGATCACAATCCAGAACCCTGACCCAGCCGTGTATCAGCT
GAGGGACTCCAAGTCCTCTGATAAGAGCGTGTGCCTGTTCACCGACTTTGATTC
TCAGACAAACGTGAGCCAGTCTAAGGACAGCGACGTGTACATCACCGACAAGTG
CGTGCTGGATATGCGCAGCATGGACTTCAAGAGCAACTCCGCCGTGGCCTGGA
GCAATAAGTCCGATTTCGCCTGCGCCAACGCCTTTAACAATTCCATCATCCCAGA
GGATACATTCTTTCCCTCTCCTGAGAGCTCCTGTGACGTGAAGCTGGTGGAGAA
GAGCTTCGAGACCGATACAAACCTGAATTTTCAGAACCTGTCCGTGATCGGCTT
CCGGATCCTGCTGCTGAAGGTGGCCGGCTTTAATCTGCTGATGACCCTGAGACT
GTGGTCTAGCAGGGCCAAGCGCGGCAGCGGCGAGGGCAGAGGAAGTCTGCTG
ACATGCGGAGACGTGGAGGAGAATCCTGGACCTATGTCCATCGGACTGCTGTG
CTGTGCCGCCCTGAGCCTGCTGTGGGCCGGCCCAGTGAACGCAGGAGTGGAG
GTGAAGCTGCAGGAGTCCAAGTTCGGACTGGTGGCACCTTCTCAGAGCCTGTC
CGTGACCTGCACAGTGTCTGGCGTGAGCCTGCCAGACTACGGCGTGAGCTGGA
TCAGGCAGCCACCTAGAAAGGGACTGGAGTGGCTGGGCGTGATCTGGGGCTCC
GAGACCACATACTATAATAGCGCCCTGAAGTCCCGGCTGACCATCATCAAGGAT
AACTCTAAGAGCCAGGTGTTTCTGAAGATGAATAGCCTGCAGACCGACGATACA
GCCATCTACTATTGCGCCAAGCACTACTATTACGGCGGCTCTTATGCCATGGACT
ACTGGGGCCAGGGCACCAGCGTGACAGTGTCCTCTGATCTGAAGAACGTGTTC
CCACCCGAGGTGGCCGTGTTTGAGCCTTCTGAGGCCGAGATCAGCCACACCCA
GAAGGCCACCCTGGTGTGCCTGGCAACCGGCTTCTATCCAGACCACGTGGAGC
TGAGCTGGTGGGTGAACGGCAAGGAGGTGCACTCCGGCGTGTGCACAGACCCT
CAGCCACTGAAGGAGCAGCCCGCCCTGAATGATTCCCGGTATTGTCTGAGCTCC
CGGCTGAGAGTGTCTGCCACCTTTTGGCAGAACCCTAGGAATCACTTCCGCTGC
CAGGTGCAGTTTTACGGCCTGAGCGAAAAcGATGAaTGGACaCAGGACAGGGCC
AAGCCCGTGACACAGATCGTGTCCGCCGAGGCATGGGGAAGGGCAGACTGTGG
CTTCACCTCCGAGTCTTACCAGCAGGGCGTGCTGAGCGCCACCATCCTGTATGA
GATCCTGCTGGGCAAGGCCACACTGTACGCCGTGCTGGTGTCTGCCCTGGTGC
TGATGGCCATGGTGAAGCGGAAGGATTCCAGAGGCCGGACCGATGGA
JFC031: miTCR1_α_PD_ß_wt (SEQ ID NO: 4)
gtcgacgttaacgccgccaccATGGAGACCCTGCTGGGCCTGCTGATCCTGTGGCTGCAG
CTGCAGTGGGTGAGCTCCAAGCAGGACATCCAGATGACCCAGACCACATCTAG
CCTGTCTGCCAGCCTGGGCGATAGGGTGACAATCTCCTGCCGCGCCTCTCAGG
ACATCAGCAAGTATCTGAACTGGTACCAGCAGAAGCCAGATGGCACCGTGAAGC
TGCTGATCTATCACACATCCCGGCTGCACTCTGGCGTGCCCAGCAGATTTTCCG
GATCTGGAAGCGGAACCGACTACTCCCTGACAATCTCTAACCTGGAGCAGGAGG
ATATCGCCACCTATTTTTGTCAGCAGGGCAATACCCTGCCTTACACATTCGGCGG
CGGCACCAAGCTGGAGATCACACCCGACATCCAGAACCCTGACCCAGCCGTGT
ATCAGCTGAGGGACTCCAAGTCCTCTGATAAGAGCGTGTGCCTGTTCACCGACT
TTGATTCTCAGACAAACGTGAGCCAGTCTAAGGACAGCGACGTGTACATCACCG
ACAAGTGCGTGCTGGATATGCGCAGCATGGACTTCAAGAGCAACTCCGCCGTG
GCCTGGAGCAATAAGTCCGATTTCGCCTGCGCCAACGCCTTTAACAATTCCATC
ATCCCAGAGGATACATTCTTTCCCTCTCCTGAGAGCTCCTGTGACGTGAAGCTG
GTGGAGAAGAGCTTCGAGACCGATACAAACCTGAATTTTCAGAACCTGTCCGTG
ATCGGCTTCCGGATCCTGCTGCTGAAGGTGGCCGGCTTTAATCTGCTGATGACC
CTGAGACTGTGGTCTAGCAGGGCCAAGCGCGGCAGCGGCGAGGGCAGAGGAA
GTCTGCTGACATGCGGAGACGTGGAGGAGAATCCTGGACCTATGTCCATCGGA
CTGCTGTGCTGTGCCGCCCTGAGCCTGCTGTGGGCCGGCCCAGTGAACGCAGG
AGTGGAGGTGAAGCTGCAGGAGTCCGGCCCCGGACTGGTGGCACCTTCTCAGA
GCCTGTCCGTGACCTGCACAGTGTCTGGCGTGAGCCTGCCAGACTACGGCGTG
AGCTGGATCAGGCAGCCACCTAGAAAGGGACTGGAGTGGCTGGGCGTGATCTG
GGGCTCCGAGACCACATACTATAATAGCGCCCTGAAGTCCCGGCTGACCATCAT
CAAGGATAACTCTAAGAGCCAGGTGTTTCTGAAGATGAATAGCCTGCAGACCGA
CGATACAGCCATCTACTATTGCGCCAAGCACTACTATTACGGCGGCTCTTATGCC
ATGGACTACTGGGGCCAGGGCACCAGCGTGACAGTGTCCTCTGATCTGAAGAA
CGTGTTCCCACCCGAGGTGGCCGTGTTTGAGCCTTCTGAGGCCGAGATCAGCC
ACACCCAGAAGGCCACCCTGGTGTGCCTGGCAACCGGCTTCTATCCAGACCAC
GTGGAGCTGAGCTGGTGGGTGAACGGCAAGGAGGTGCACTCCGGCGTGTGCA
CAGACCCTCAGCCACTGAAGGAGCAGCCCGCCCTGAATGATTCCCGGTATTGTC
TGAGCTCCCGGCTGAGAGTGTCTGCCACCTTTTGGCAGAACCCTAGGAATCACT
TCCGCTGCCAGGTGCAGTTTTACGGCCTGAGCGAAAAcGATGAaTGGACaCAGG
ACAGGGCCAAGCCCGTGACACAGATCGTGTCCGCCGAGGCATGGGGAAGGGC
AGACTGTGGCTTCACCTCCGAGTCTTACCAGCAGGGCGTGCTGAGCGCCACCA
TCCTGTATGAGATCCTGCTGGGCAAGGCCACACTGTACGCCGTGCTGGTGTCTG
CCCTGGTGCTGATGGCCATGGTGAAGCGGAAGGATTCCAGAGGCCGGACCGAT
GGA
JFC032: miTCR1_α_PD_β_G31K_P32F (SEQ ID NO: 5)
gtcgacgttaacgccgccaccATGGAGACCCTGCTGGGCCTGCTGATCCTGTGGCTGCAG
CTGCAGTGGGTGAGCTCCAAGCAGGACATCCAGATGACCCAGACCACATCTAG
CCTGTCTGCCAGCCTGGGCGATAGGGTGACAATCTCCTGCCGCGCCTCTCAGG
ACATCAGCAAGTATCTGAACTGGTACCAGCAGAAGCCAGATGGCACCGTGAAGC
TGCTGATCTATCACACATCCCGGCTGCACTCTGGCGTGCCCAGCAGATTTTCCG
GATCTGGAAGCGGAACCGACTACTCCCTGACAATCTCTAACCTGGAGCAGGAGG
ATATCGCCACCTATTTTTGTCAGCAGGGCAATACCCTGCCTTACACATTCGGCGG
CGGCACCAAGCTGGAGATCACACCCGACATCCAGAACCCTGACCCAGCCGTGT
ATCAGCTGAGGGACTCCAAGTCCTCTGATAAGAGCGTGTGCCTGTTCACCGACT
TTGATTCTCAGACAAACGTGAGCCAGTCTAAGGACAGCGACGTGTACATCACCG
ACAAGTGCGTGCTGGATATGCGCAGCATGGACTTCAAGAGCAACTCCGCCGTG
GCCTGGAGCAATAAGTCCGATTTCGCCTGCGCCAACGCCTTTAACAATTCCATC
ATCCCAGAGGATACATTCTTTCCCTCTCCTGAGAGCTCCTGTGACGTGAAGCTG
GTGGAGAAGAGCTTCGAGACCGATACAAACCTGAATTTTCAGAACCTGTCCGTG
ATCGGCTTCCGGATCCTGCTGCTGAAGGTGGCCGGCTTTAATCTGCTGATGACC
CTGAGACTGTGGTCTAGCAGGGCCAAGCGCGGCAGCGGCGAGGGCAGAGGAA
GTCTGCTGACATGCGGAGACGTGGAGGAGAATCCTGGACCTATGTCCATCGGA
CTGCTGTGCTGTGCCGCCCTGAGCCTGCTGTGGGCCGGCCCAGTGAACGCAGG
AGTGGAGGTGAAGCTGCAGGAGTCCAAGTTCGGACTGGTGGCACCTTCTCAGA
GCCTGTCCGTGACCTGCACAGTGTCTGGCGTGAGCCTGCCAGACTACGGCGTG
AGCTGGATCAGGCAGCCACCTAGAAAGGGACTGGAGTGGCTGGGCGTGATCTG
GGGCTCCGAGACCACATACTATAATAGCGCCCTGAAGTCCCGGCTGACCATCAT
CAAGGATAACTCTAAGAGCCAGGTGTTTCTGAAGATGAATAGCCTGCAGACCGA
CGATACAGCCATCTACTATTGCGCCAAGCACTACTATTACGGCGGCTCTTATGCC
ATGGACTACTGGGGCCAGGGCACCAGCGTGACAGTGTCCTCTGATCTGAAGAA
CGTGTTCCCACCCGAGGTGGCCGTGTTTGAGCCTTCTGAGGCCGAGATCAGCC
ACACCCAGAAGGCCACCCTGGTGTGCCTGGCAACCGGCTTCTATCCAGACCAC
GTGGAGCTGAGCTGGTGGGTGAACGGCAAGGAGGTGCACTCCGGCGTGTGCA
CAGACCCTCAGCCACTGAAGGAGCAGCCCGCCCTGAATGATTCCCGGTATTGTC
TGAGCTCCCGGCTGAGAGTGTCTGCCACCTTTTGGCAGAACCCTAGGAATCACT
TCCGCTGCCAGGTGCAGTTTTACGGCCTGAGCGAAAAcGATGAaTGGACaCAGG
ACAGGGCCAAGCCCGTGACACAGATCGTGTCCGCCGAGGCATGGGGAAGGGC
AGACTGTGGCTTCACCTCCGAGTCTTACCAGCAGGGCGTGCTGAGCGCCACCA
TCCTGTATGAGATCCTGCTGGGCAAGGCCACACTGTACGCCGTGCTGGTGTCTG
CCCTGGTGCTGATGGCCATGGTGAAGCGGAAGGATTCCAGAGGCCGGACCGAT
GGA
JFC033: miTCR1_α_Q101L_β_wt (SEQ ID NO: 6)
gtcgacgttaacgccgccaccATGGAGACCCTGCTGGGCCTGCTGATCCTGTGGCTGCAG
CTGCAGTGGGTGAGCTCCAAGCAGGACATCCAGATGACCCAGACCACATCTAG
CCTGTCTGCCAGCCTGGGCGATAGGGTGACAATCTCCTGCCGCGCCTCTCAGG
ACATCAGCAAGTATCTGAACTGGTACCAGCAGAAGCCAGATGGCACCGTGAAGC
TGCTGATCTATCACACATCCCGGCTGCACTCTGGCGTGCCCAGCAGATTTTCCG
GATCTGGAAGCGGAACCGACTACTCCCTGACAATCTCTAACCTGGAGctgGAGGA
TATCGCCACCTATTTTTGTCAGCAGGGCAATACCCTGCCTTACACATTCGGCGG
CGGCACCAAGCTGGAGATCACAATCCAGAACCCTGACCCAGCCGTGTATCAGCT
GAGGGACTCCAAGTCCTCTGATAAGAGCGTGTGCCTGTTCACCGACTTTGATTC
TCAGACAAACGTGAGCCAGTCTAAGGACAGCGACGTGTACATCACCGACAAGTG
CGTGCTGGATATGCGCAGCATGGACTTCAAGAGCAACTCCGCCGTGGCCTGGA
GCAATAAGTCCGATTTCGCCTGCGCCAACGCCTTTAACAATTCCATCATCCCAGA
GGATACATTCTTTCCCTCTCCTGAGAGCTCCTGTGACGTGAAGCTGGTGGAGAA
GAGCTTCGAGACCGATACAAACCTGAATTTTCAGAACCTGTCCGTGATCGGCTT
CCGGATCCTGCTGCTGAAGGTGGCCGGCTTTAATCTGCTGATGACCCTGAGACT
GTGGTCTAGCAGGGCCAAGCGCGGCAGCGGCGAGGGCAGAGGAAGTCTGCTG
ACATGCGGAGACGTGGAGGAGAATCCTGGACCTATGTCCATCGGACTGCTGTG
CTGTGCCGCCCTGAGCCTGCTGTGGGCCGGCCCAGTGAACGCAGGAGTGGAG
GTGAAGCTGCAGGAGTCCGGCCCCGGACTGGTGGCACCTTCTCAGAGCCTGTC
CGTGACCTGCACAGTGTCTGGCGTGAGCCTGCCAGACTACGGCGTGAGCTGGA
TCAGGCAGCCACCTAGAAAGGGACTGGAGTGGCTGGGCGTGATCTGGGGCTCC
GAGACCACATACTATAATAGCGCCCTGAAGTCCCGGCTGACCATCATCAAGGAT
AACTCTAAGAGCCAGGTGTTTCTGAAGATGAATAGCCTGCAGACCGACGATACA
GCCATCTACTATTGCGCCAAGCACTACTATTACGGCGGCTCTTATGCCATGGACT
ACTGGGGCCAGGGCACCAGCGTGACAGTGTCCTCTGATCTGAAGAACGTGTTC
CCACCCGAGGTGGCCGTGTTTGAGCCTTCTGAGGCCGAGATCAGCCACACCCA
GAAGGCCACCCTGGTGTGCCTGGCAACCGGCTTCTATCCAGACCACGTGGAGC
TGAGCTGGTGGGTGAACGGCAAGGAGGTGCACTCCGGCGTGTGCACAGACCCT
CAGCCACTGAAGGAGCAGCCCGCCCTGAATGATTCCCGGTATTGTCTGAGCTCC
CGGCTGAGAGTGTCTGCCACCTTTTGGCAGAACCCTAGGAATCACTTCCGCTGC
CAGGTGCAGTTTTACGGCCTGAGCGAAAAcGATGAATGGACaCAGGACAGGGCC
AAGCCCGTGACACAGATCGTGTCCGCCGAGGCATGGGGAAGGGCAGACTGTGG
CTTCACCTCCGAGTCTTACCAGCAGGGCGTGCTGAGCGCCACCATCCTGTATGA
GATCCTGCTGGGCAAGGCCACACTGTACGCCGTGCTGGTGTCTGCCCTGGTGC
TGATGGCCATGGTGAAGCGGAAGGATTCCAGAGGCCGGACCGATGGA
JFC034: miTCR1_α_PD_Q101L_β_wt (SEQ ID NO: 7)
gtcgacgttaacgccgccaccATGGAGACCCTGCTGGGCCTGCTGATCCTGTGGCTGCAG
CTGCAGTGGGTGAGCTCCAAGCAGGACATCCAGATGACCCAGACCACATCTAG
CCTGTCTGCCAGCCTGGGCGATAGGGTGACAATCTCCTGCCGCGCCTCTCAGG
ACATCAGCAAGTATCTGAACTGGTACCAGCAGAAGCCAGATGGCACCGTGAAGC
TGCTGATCTATCACACATCCCGGCTGCACTCTGGCGTGCCCAGCAGATTTTCCG
GATCTGGAAGCGGAACCGACTACTCCCTGACAATCTCTAACCTGGAGctgGAGGA
TATCGCCACCTATTTTTGTCAGCAGGGCAATACCCTGCCTTACACATTCGGCGG
CGGCACCAAGCTGGAGATCACACCCGACATCCAGAACCCTGACCCAGCCGTGT
ATCAGCTGAGGGACTCCAAGTCCTCTGATAAGAGCGTGTGCCTGTTCACCGACT
TTGATTCTCAGACAAACGTGAGCCAGTCTAAGGACAGCGACGTGTACATCACCG
ACAAGTGCGTGCTGGATATGCGCAGCATGGACTTCAAGAGCAACTCCGCCGTG
GCCTGGAGCAATAAGTCCGATTTCGCCTGCGCCAACGCCTTTAACAATTCCATC
ATCCCAGAGGATACATTCTTTCCCTCTCCTGAGAGCTCCTGTGACGTGAAGCTG
GTGGAGAAGAGCTTCGAGACCGATACAAACCTGAATTTTCAGAACCTGTCCGTG
ATCGGCTTCCGGATCCTGCTGCTGAAGGTGGCCGGCTTTAATCTGCTGATGACC
CTGAGACTGTGGTCTAGCAGGGCCAAGCGCGGCAGCGGCGAGGGCAGAGGAA
GTCTGCTGACATGCGGAGACGTGGAGGAGAATCCTGGACCTATGTCCATCGGA
CTGCTGTGCTGTGCCGCCCTGAGCCTGCTGTGGGCCGGCCCAGTGAACGCAGG
AGTGGAGGTGAAGCTGCAGGAGTCCGGCCCCGGACTGGTGGCACCTTCTCAGA
GCCTGTCCGTGACCTGCACAGTGTCTGGCGTGAGCCTGCCAGACTACGGCGTG
AGCTGGATCAGGCAGCCACCTAGAAAGGGACTGGAGTGGCTGGGCGTGATCTG
GGGCTCCGAGACCACATACTATAATAGCGCCCTGAAGTCCCGGCTGACCATCAT
CAAGGATAACTCTAAGAGCCAGGTGTTTCTGAAGATGAATAGCCTGCAGACCGA
CGATACAGCCATCTACTATTGCGCCAAGCACTACTATTACGGCGGCTCTTATGCC
ATGGACTACTGGGGCCAGGGCACCAGCGTGACAGTGTCCTCTGATCTGAAGAA
CGTGTTCCCACCCGAGGTGGCCGTGTTTGAGCCTTCTGAGGCCGAGATCAGCC
ACACCCAGAAGGCCACCCTGGTGTGCCTGGCAACCGGCTTCTATCCAGACCAC
GTGGAGCTGAGCTGGTGGGTGAACGGCAAGGAGGTGCACTCCGGCGTGTGCA
CAGACCCTCAGCCACTGAAGGAGCAGCCCGCCCTGAATGATTCCCGGTATTGTC
TGAGCTCCCGGCTGAGAGTGTCTGCCACCTTTTGGCAGAACCCTAGGAATCACT
TCCGCTGCCAGGTGCAGTTTTACGGCCTGAGCGAAAAcGATGAaTGGACaCAGG
ACAGGGCCAAGCCCGTGACACAGATCGTGTCCGCCGAGGCATGGGGAAGGGC
AGACTGTGGCTTCACCTCCGAGTCTTACCAGCAGGGCGTGCTGAGCGCCACCA
TCCTGTATGAGATCCTGCTGGGCAAGGCCACACTGTACGCCGTGCTGGTGTCTG
CCCTGGTGCTGATGGCCATGGTGAAGCGGAAGGATTCCAGAGGCCGGACCGAT
JFC035: miTCR1_α_PD_Q101L_L36E_β_wt (SEQ ID NO: 8)
gtcgacgttaacgccgccaccATGGAGACCCTGCTGGGCCTGCTGATCCTGTGGCTGCAG
CTGCAGTGGGTGAGCTCCAAGCAGGACATCCAGATGACCCAGACCACATCTAG
CCTGTCTGCCAGCgagGGCGATAGGGTGACAATCTCCTGCCGCGCCTCTCAGGA
CATCAGCAAGTATCTGAACTGGTACCAGCAGAAGCCAGATGGCACCGTGAAGCT
GCTGATCTATCACACATCCCGGCTGCACTCTGGCGTGCCCAGCAGATTTTCCGG
ATCTGGAAGCGGAACCGACTACTCCCTGACAATCTCTAACCTGGAGctgGAGGAT
ATCGCCACCTATTTTTGTCAGCAGGGCAATACCCTGCCTTACACATTCGGCGGC
GGCACCAAGCTGGAGATCACACCCGACATCCAGAACCCTGACCCAGCCGTGTA
TCAGCTGAGGGACTCCAAGTCCTCTGATAAGAGCGTGTGCCTGTTCACCGACTT
TGATTCTCAGACAAACGTGAGCCAGTCTAAGGACAGCGACGTGTACATCACCGA
CAAGTGCGTGCTGGATATGCGCAGCATGGACTTCAAGAGCAACTCCGCCGTGG
CCTGGAGCAATAAGTCCGATTTCGCCTGCGCCAACGCCTTTAACAATTCCATCAT
CCCAGAGGATACATTCTTTCCCTCTCCTGAGAGCTCCTGTGACGTGAAGCTGGT
GGAGAAGAGCTTCGAGACCGATACAAACCTGAATTTTCAGAACCTGTCCGTGAT
CGGCTTCCGGATCCTGCTGCTGAAGGTGGCCGGCTTTAATCTGCTGATGACCCT
GAGACTGTGGTCTAGCAGGGCCAAGCGCGGCAGCGGCGAGGGCAGAGGAAGT
CTGCTGACATGCGGAGACGTGGAGGAGAATCCTGGACCTATGTCCATCGGACT
GCTGTGCTGTGCCGCCCTGAGCCTGCTGTGGGCCGGCCCAGTGAACGCAGGA
GTGGAGGTGAAGCTGCAGGAGTCCGGCCCCGGACTGGTGGCACCTTCTCAGAG
CCTGTCCGTGACCTGCACAGTGTCTGGCGTGAGCCTGCCAGACTACGGCGTGA
GCTGGATCAGGCAGCCACCTAGAAAGGGACTGGAGTGGCTGGGCGTGATCTGG
GGCTCCGAGACCACATACTATAATAGCGCCCTGAAGTCCCGGCTGACCATCATC
AAGGATAACTCTAAGAGCCAGGTGTTTCTGAAGATGAATAGCCTGCAGACCGAC
GATACAGCCATCTACTATTGCGCCAAGCACTACTATTACGGCGGCTCTTATGCCA
TGGACTACTGGGGCCAGGGCACCAGCGTGACAGTGTCCTCTGATCTGAAGAAC
GTGTTCCCACCCGAGGTGGCCGTGTTTGAGCCTTCTGAGGCCGAGATCAGCCA
CACCCAGAAGGCCACCCTGGTGTGCCTGGCAACCGGCTTCTATCCAGACCACG
TGGAGCTGAGCTGGTGGGTGAACGGCAAGGAGGTGCACTCCGGCGTGTGCACA
GACCCTCAGCCACTGAAGGAGCAGCCCGCCCTGAATGATTCCCGGTATTGTCTG
AGCTCCCGGCTGAGAGTGTCTGCCACCTTTTGGCAGAACCCTAGGAATCACTTC
CGCTGCCAGGTGCAGTTTTACGGCCTGAGCGAAAAGATGAaTGGACaCAGGAC
AGGGCCAAGCCCGTGACACAGATCGTGTCCGCCGAGGCATGGGGAAGGGCAG
ACTGTGGCTTCACCTCCGAGTCTTACCAGCAGGGCGTGCTGAGCGCCACCATC
CTGTATGAGATCCTGCTGGGCAAGGCCACACTGTACGCCGTGCTGGTGTCTGC
CCTGGTGCTGATGGCCATGGTGAAGCGGAAGGATTCCAGAGGCCGGACCGAT
JFC036: miTCR1_α_Q101L_β_G31K_P32F (SEQ ID NO: 9)
gtcgacgttaacgccgccaccATGGAGACCCTGCTGGGCCTGCTGATCCTGTGGCTGCAG
CTGCAGTGGGTGAGCTCCAAGCAGGACATCCAGATGACCCAGACCACATCTAG
CCTGTCTGCCAGCCTGGGCGATAGGGTGACAATCTCCTGCCGCGCCTCTCAGG
ACATCAGCAAGTATCTGAACTGGTACCAGCAGAAGCCAGATGGCACCGTGAAGC
TGCTGATCTATCACACATCCCGGCTGCACTCTGGCGTGCCCAGCAGATTTTCCG
GATCTGGAAGCGGAACCGACTACTCCCTGACAATCTCTAACCTGGAGctgGAGGA
TATCGCCACCTATTTTTGTCAGCAGGGCAATACCCTGCCTTACACATTCGGCGG
CGGCACCAAGCTGGAGATCACAATCCAGAACCCTGACCCAGCCGTGTATCAGCT
GAGGGACTCCAAGTCCTCTGATAAGAGCGTGTGCCTGTTCACCGACTTTGATTC
TCAGACAAACGTGAGCCAGTCTAAGGACAGCGACGTGTACATCACCGACAAGTG
CGTGCTGGATATGCGCAGCATGGACTTCAAGAGCAACTCCGCCGTGGCCTGGA
GCAATAAGTCCGATTTCGCCTGCGCCAACGCCTTTAACAATTCCATCATCCCAGA
GGATACATTCTTTCCCTCTCCTGAGAGCTCCTGTGACGTGAAGCTGGTGGAGAA
GAGCTTCGAGACCGATACAAACCTGAATTTTCAGAACCTGTCCGTGATCGGCTT
CCGGATCCTGCTGCTGAAGGTGGCCGGCTTTAATCTGCTGATGACCCTGAGACT
GTGGTCTAGCAGGGCCAAGCGCGGCAGCGGCGAGGGCAGAGGAAGTCTGCTG
ACATGCGGAGACGTGGAGGAGAATCCTGGACCTATGTCCATCGGACTGCTGTG
CTGTGCCGCCCTGAGCCTGCTGTGGGCCGGCCCAGTGAACGCAGGAGTGGAG
GTGAAGCTGCAGGAGTCCaagTTCGGACTGGTGGCACCTTCTCAGAGCCTGTCC
GTGACCTGCACAGTGTCTGGCGTGAGCCTGCCAGACTACGGCGTGAGCTGGAT
CAGGCAGCCACCTAGAAAGGGACTGGAGTGGCTGGGCGTGATCTGGGGCTCC
GAGACCACATACTATAATAGCGCCCTGAAGTCCCGGCTGACCATCATCAAGGAT
AACTCTAAGAGCCAGGTGTTTCTGAAGATGAATAGCCTGCAGACCGACGATACA
GCCATCTACTATTGCGCCAAGCACTACTATTACGGCGGCTCTTATGCCATGGACT
ACTGGGGCCAGGGCACCAGCGTGACAGTGTCCTCTGATCTGAAGAACGTGTTC
CCACCCGAGGTGGCCGTGTTTGAGCCTTCTGAGGCCGAGATCAGCCACACCCA
GAAGGCCACCCTGGTGTGCCTGGCAACCGGCTTCTATCCAGACCACGTGGAGC
TGAGCTGGTGGGTGAACGGCAAGGAGGTGCACTCCGGCGTGTGCACAGACCCT
CAGCCACTGAAGGAGCAGCCCGCCCTGAATGATTCCCGGTATTGTCTGAGCTCC
CGGCTGAGAGTGTCTGCCACCTTTTGGCAGAACCCTAGGAATCACTTCCGCTGC
CAGGTGCAGTTTTACGGCCTGAGCGAAAAcGATGAaTGGACaCAGGACAGGGCC
AAGCCCGTGACACAGATCGTGTCCGCCGAGGCATGGGGAAGGGCAGACTGTGG
CTTCACCTCCGAGTCTTACCAGCAGGGCGTGCTGAGCGCCACCATCCTGTATGA
GATCCTGCTGGGCAAGGCCACACTGTACGCCGTGCTGGTGTCTGCCCTGGTGC
TGATGGCCATGGTGAAGCGGAAGGATTCCAGAGGCCGGACCGATGGAAGCGGA
GCTACTAACTTCAGCCTGCTGAAGCAGGCTGGAGACGTGGAGGA
JFC037: miTCR1_α_PD_Q101L_β_G31K_P32F (SEQ ID NO: 10)
gtcgacgttaacgccgccaccATGGAGACCCTGCTGGGCCTGCTGATCCTGTGGCTGCAG
CTGCAGTGGGTGAGCTCCAAGCAGGACATCCAGATGACCCAGACCACATCTAG
CCTGTCTGCCAGCCTGGGCGATAGGGTGACAATCTCCTGCCGCGCCTCTCAGG
ACATCAGCAAGTATCTGAACTGGTACCAGCAGAAGCCAGATGGCACCGTGAAGC
TGCTGATCTATCACACATCCCGGCTGCACTCTGGCGTGCCCAGCAGATTTTCCG
GATCTGGAAGCGGAACCGACTACTCCCTGACAATCTCTAACCTGGAGctgGAGGA
TATCGCCACCTATTTTTGTCAGCAGGGCAATACCCTGCCTTACACATTCGGCGG
CGGCACCAAGCTGGAGATCACACCCGACATCCAGAACCCTGACCCAGCCGTGT
ATCAGCTGAGGGACTCCAAGTCCTCTGATAAGAGCGTGTGCCTGTTCACCGACT
TTGATTCTCAGACAAACGTGAGCCAGTCTAAGGACAGCGACGTGTACATCACCG
ACAAGTGCGTGCTGGATATGCGCAGCATGGACTTCAAGAGCAACTCCGCCGTG
GCCTGGAGCAATAAGTCCGATTTCGCCTGCGCCAACGCCTTTAACAATTCCATC
ATCCCAGAGGATACATTCTTTCCCTCTCCTGAGAGCTCCTGTGACGTGAAGCTG
GTGGAGAAGAGCTTCGAGACCGATACAAACCTGAATTTTCAGAACCTGTCCGTG
ATCGGCTTCCGGATCCTGCTGCTGAAGGTGGCCGGCTTTAATCTGCTGATGACC
CTGAGACTGTGGTCTAGCAGGGCCAAGCGCGGCAGCGGCGAGGGCAGAGGAA
GTCTGCTGACATGCGGAGACGTGGAGGAGAATCCTGGACCTATGTCCATCGGA
CTGCTGTGCTGTGCCGCCCTGAGCCTGCTGTGGGCCGGCCCAGTGAACGCAGG
AGTGGAGGTGAAGCTGCAGGAGTCCaagTTCGGACTGGTGGCACCTTCTCAGAG
CCTGTCCGTGACCTGCACAGTGTCTGGCGTGAGCCTGCCAGACTACGGCGTGA
GCTGGATCAGGCAGCCACCTAGAAAGGGACTGGAGTGGCTGGGCGTGATCTGG
GGCTCCGAGACCACATACTATAATAGCGCCCTGAAGTCCCGGCTGACCATCATC
AAGGATAACTCTAAGAGCCAGGTGTTTCTGAAGATGAATAGCCTGCAGACCGAC
GATACAGCCATCTACTATTGCGCCAAGCACTACTATTACGGCGGCTCTTATGCCA
TGGACTACTGGGGCCAGGGCACCAGCGTGACAGTGTCCTCTGATCTGAAGAAC
GTGTTCCCACCCGAGGTGGCCGTGTTTGAGCCTTCTGAGGCCGAGATCAGCCA
CACCCAGAAGGCCACCCTGGTGTGCCTGGCAACCGGCTTCTATCCAGACCACG
TGGAGCTGAGCTGGTGGGTGAACGGCAAGGAGGTGCACTCCGGCGTGTGCACA
GACCCTCAGCCACTGAAGGAGCAGCCCGCCCTGAATGATTCCCGGTATTGTCTG
AGCTCCCGGCTGAGAGTGTCTGCCACCTTTTGGCAGAACCCTAGGAATCACTTC
CGCTGCCAGGTGCAGTTTTACGGCCTGAGCGAAAAcGATGAATGGACaCAGGAC
AGGGCCAAGCCCGTGACACAGATCGTGTCCGCCGAGGCATGGGGAAGGGCAG
ACTGTGGCTTCACCTCCGAGTCTTACCAGCAGGGCGTGCTGAGCGCCACCATC
CTGTATGAGATCCTGCTGGGCAAGGCCACACTGTACGCCGTGCTGGTGTCTGC
CCTGGTGCTGATGGCCATGGTGAAGCGGAAGGATTCCAGAGGCCGGACCGATG
JFC038: miTCR1_α_PD_Q101L_L36E_β_G31K_P32F (SEQ ID NO: 11)
gtcgacgttaacgccgccaccATGGAGACCCTGCTGGGCCTGCTGATCCTGTGGCTGCAG
CTGCAGTGGGTGAGCTCCAAGCAGGACATCCAGATGACCCAGACCACATCTAG
CCTGTCTGCCAGCgagGGCGATAGGGTGACAATCTCCTGCCGCGCCTCTCAGGA
CATCAGCAAGTATCTGAACTGGTACCAGCAGAAGCCAGATGGCACCGTGAAGCT
GCTGATCTATCACACATCCCGGCTGCACTCTGGCGTGCCCAGCAGATTTTCCGG
ATCTGGAAGCGGAACCGACTACTCCCTGACAATCTCTAACCTGGAGctgGAGGAT
ATCGCCACCTATTTTTGTCAGCAGGGCAATACCCTGCCTTACACATTCGGCGGC
GGCACCAAGCTGGAGATCACACCCGACATCCAGAACCCTGACCCAGCCGTGTA
TCAGCTGAGGGACTCCAAGTCCTCTGATAAGAGCGTGTGCCTGTTCACCGACTT
TGATTCTCAGACAAACGTGAGCCAGTCTAAGGACAGCGACGTGTACATCACCGA
CAAGTGCGTGCTGGATATGCGCAGCATGGACTTCAAGAGCAACTCCGCCGTGG
CCTGGAGCAATAAGTCCGATTTCGCCTGCGCCAACGCCTTTAACAATTCCATCAT
CCCAGAGGATACATTCTTTCCCTCTCCTGAGAGCTCCTGTGACGTGAAGCTGGT
GGAGAAGAGCTTCGAGACCGATACAAACCTGAATTTTCAGAACCTGTCCGTGAT
CGGCTTCCGGATCCTGCTGCTGAAGGTGGCCGGCTTTAATCTGCTGATGACCCT
GAGACTGTGGTCTAGCAGGGCCAAGCGCGGCAGCGGCGAGGGCAGAGGAAGT
CTGCTGACATGCGGAGACGTGGAGGAGAATCCTGGACCTATGTCCATCGGACT
GCTGTGCTGTGCCGCCCTGAGCCTGCTGTGGGCCGGCCCAGTGAACGCAGGA
GTGGAGGTGAAGCTGCAGGAGTCCaagTTCGGACTGGTGGCACCTTCTCAGAGC
CTGTCCGTGACCTGCACAGTGTCTGGCGTGAGCCTGCCAGACTACGGCGTGAG
CTGGATCAGGCAGCCACCTAGAAAGGGACTGGAGTGGCTGGGCGTGATCTGGG
GCTCCGAGACCACATACTATAATAGCGCCCTGAAGTCCCGGCTGACCATCATCA
AGGATAACTCTAAGAGCCAGGTGTTTCTGAAGATGAATAGCCTGCAGACCGACG
ATACAGCCATCTACTATTGCGCCAAGCACTACTATTACGGCGGCTCTTATGCCAT
GGACTACTGGGGCCAGGGCACCAGCGTGACAGTGTCCTCTGATCTGAAGAACG
TGTTCCCACCCGAGGTGGCCGTGTTTGAGCCTTCTGAGGCCGAGATCAGCCAC
ACCCAGAAGGCCACCCTGGTGTGCCTGGCAACCGGCTTCTATCCAGACCACGT
GGAGCTGAGCTGGTGGGTGAACGGCAAGGAGGTGCACTCCGGCGTGTGCACA
GACCCTCAGCCACTGAAGGAGCAGCCCGCCCTGAATGATTCCCGGTATTGTCTG
AGCTCCCGGCTGAGAGTGTCTGCCACCTTTTGGCAGAACCCTAGGAATCACTTC
CGCTGCCAGGTGCAGTTTTACGGCCTGAGCGAAAAcGATGAATGGACaCAGGAC
AGGGCCAAGCCCGTGACACAGATCGTGTCCGCCGAGGCATGGGGAAGGGCAG
ACTGTGGCTTCACCTCCGAGTCTTACCAGCAGGGCGTGCTGAGCGCCACCATC
CTGTATGAGATCCTGCTGGGCAAGGCCACACTGTACGCCGTGCTGGTGTCTGC
CCTGGTGCTGATGGCCATGGTGAAGCGGAAGGATTCCAGAGGCCGGACCGATG
JFC040: miTCR2_α_wt_β_S32K_S33F (SEQ ID NO: 12)
gtcgacgttaacgccgccaccATGGAGACCCTGCTGGGCCTGCTGATCCTGTGGCTGCAG
CTGCAGTGGGTGAGCTCCAAGCAGGAGGTGAAGCTGCAGGAGAGCGGCCCCG
GACTGGTGGCACCTTCTCAGAGCCTGTCCGTGACCTGCACAGTGAGCGGCGTG
TCCCTGCCTGACTACGGCGTGAGCTGGATCAGGCAGCCACCTAGAAAGGGACT
GGAGTGGCTGGGCGTGATCTGGGGCTCCGAGACCACATACTATAACTCCGCCC
TGAAGTCTCGGCTGACCATCATCAAGGATAACTCTAAGAGCCAGGTGTTTCTGAA
GATGAATTCCCTGCAGACCGACGATACAGCCATCTACTATTGTGCCAAGCACTA
CTATTACGGCGGCTCTTATGCCATGGACTACTGGGGCCAGGGCACCAGCGTGA
CAGTGTCTAGCATCCAGAACCCTGATCCAGCCGTGTATCAGCTGAGGGACTCCA
AGTCCTCTGATAAGAGCGTGTGCCTGTTCACCGACTTTGATTCTCAGACAAACGT
GAGCCAGTCTAAGGACAGCGACGTGTACATCACCGACAAGTGCGTGCTGGATAT
GCGCAGCATGGACTTCAAGAGCAACTCCGCCGTGGCCTGGTCTAATAAGAGCG
ACTTCGCCTGCGCCAACGCCTTTAACAATTCCATCATCCCAGAGGATACATTCTT
TCCCTCTCCTGAGAGCTCCTGTGACGTGAAGCTGGTGGAGAAGAGCTTCGAGAC
CGATACAAACCTGAATTTTCAGAACCTGTCCGTGATCGGCTTCCGGATCCTGCT
GCTGAAGGTGGCCGGCTTTAATCTGCTGATGACCCTGAGACTGTGGTCTAGCAG
GGCCAAGCGCGGCAGCGGCGAGGGCAGAGGAAGTCTGCTGACATGCGGAGAC
GTGGAGGAGAATCCTGGACCTATGTCCATCGGACTGCTGTGCTGTGCCGCCCT
GAGCCTGCTGTGGGCCGGCCCAGTGAACGCAGGCGTGGACATCCAGATGACCC
AGACCACAAAGTTCCTGTCTGCCAGCCTGGGCGACAGGGTGACAATCAGCTGC
CGCGCCTCCCAGGATATCTCTAAGTATCTGAATTGGTACCAGCAGAAGCCAGAT
GGCACCGTGAAGCTGCTGATCTATCACACAAGCCGGCTGCACTCCGGAGTGCC
ATCTAGATTTTCCGGATCTGGAAGCGGAACCGACTACAGCCTGACAATCTCCAA
CCTGGAGCAGGAGGATATCGCCACCTATTTTTGCCAGCAGGGCAATACCCTGCC
CTACACATTCGGCGGCGGCACCAAGCTGGAGATCACAGACCTGAAGAACGTGTT
CCCACCCGAGGTGGCCGTGTTTGAGCCTTCTGAGGCCGAGATCAGCCACACCC
AGAAGGCCACCCTGGTGTGCCTGGCAACCGGCTTCTATCCAGATCACGTGGAG
CTGAGCTGGTGGGTGAACGGCAAGGAGGTGCACTCCGGCGTGTGCACAGACCC
TCAGCCACTGAAGGAGCAGCCCGCCCTGAATGATTCCCGGTATTGTCTGAGCTC
CCGGCTGAGAGTGTCTGCCACCTTTTGGCAGAACCCTAGGAATCACTTCCGCTG
CCAGGTGCAGTTTTACGGCCTGAGCGAAAACGATGAATGGACACAGGACAGGG
CCAAGCCCGTGACACAGATCGTGTCCGCCGAGGCATGGGGAAGGGCAGACTGT
GGCTTCACCTCCGAGTCTTACCAGCAGGGCGTGCTGAGCGCCACCATCCTGTAT
GAGATCCTGCTGGGCAAGGCCACACTGTACGCCGTGCTGGTGTCTGCCCTGGT
GCTGATGGCCATGGTGAAGCGGAAGGATTCCAGAGGCCGGACCGATGGA
JFC041: miTCR2_α_S141PD_β_wt (SEQ ID NO: 13)
gtcgacgttaacgccgccaccATGGAGACCCTGCTGGGCCTGCTGATCCTGTGGCTGCAG
CTGCAGTGGGTGAGCTCCAAGCAGGAGGTGAAGCTGCAGGAGAGCGGCCCCG
GACTGGTGGCACCTTCTCAGAGCCTGTCCGTGACCTGCACAGTGAGCGGCGTG
TCCCTGCCTGACTACGGCGTGAGCTGGATCAGGCAGCCACCTAGAAAGGGACT
GGAGTGGCTGGGCGTGATCTGGGGCTCCGAGACCACATACTATAACTCCGCCC
TGAAGTCTCGGCTGACCATCATCAAGGATAACTCTAAGAGCCAGGTGTTTCTGAA
GATGAATTCCCTGCAGACCGACGATACAGCCATCTACTATTGTGCCAAGCACTA
CTATTACGGCGGCTCTTATGCCATGGACTACTGGGGCCAGGGCACCAGCGTGA
CAGTGTCTCCCGACATCCAGAACCCTGATCCAGCCGTGTATCAGCTGAGGGACT
CCAAGTCCTCTGATAAGAGCGTGTGCCTGTTCACCGACTTTGATTCTCAGACAAA
CGTGAGCCAGTCTAAGGACAGCGACGTGTACATCACCGACAAGTGCGTGCTGG
ATATGCGCAGCATGGACTTCAAGAGCAACTCCGCCGTGGCCTGGTCTAATAAGA
GCGACTTCGCCTGCGCCAACGCCTTTAACAATTCCATCATCCCAGAGGATACATT
CTTTCCCTCTCCTGAGAGCTCCTGTGACGTGAAGCTGGTGGAGAAGAGCTTCGA
GACCGATACAAACCTGAATTTTCAGAACCTGTCCGTGATCGGCTTCCGGATCCT
GCTGCTGAAGGTGGCCGGCTTTAATCTGCTGATGACCCTGAGACTGTGGTCTAG
CAGGGCCAAGCGCGGCAGCGGCGAGGGCAGAGGAAGTCTGCTGACATGCGGA
GACGTGGAGGAGAATCCTGGACCTATGTCCATCGGACTGCTGTGCTGTGCCGC
CCTGAGCCTGCTGTGGGCCGGCCCAGTGAACGCAGGCGTGGACATCCAGATGA
CCCAGACCACATCCTCTCTGTCTGCCAGCCTGGGCGACAGGGTGACAATCAGCT
GCCGCGCCTCCCAGGATATCTCTAAGTATCTGAATTGGTACCAGCAGAAGCCAG
ATGGCACCGTGAAGCTGCTGATCTATCACACAAGCCGGCTGCACTCCGGAGTG
CCATCTAGATTTTCCGGATCTGGAAGCGGAACCGACTACAGCCTGACAATCTCC
AACCTGGAGCAGGAGGATATCGCCACCTATTTTTGCCAGCAGGGCAATACCCTG
CCCTACACATTCGGCGGCGGCACCAAGCTGGAGATCACAGACCTGAAGAACGT
GTTCCCACCCGAGGTGGCCGTGTTTGAGCCTTCTGAGGCCGAGATCAGCCACA
CCCAGAAGGCCACCCTGGTGTGCCTGGCAACCGGCTTCTATCCAGATCACGTG
GAGCTGAGCTGGTGGGTGAACGGCAAGGAGGTGCACTCCGGCGTGTGCACAG
ACCCTCAGCCACTGAAGGAGCAGCCCGCCCTGAATGATTCCCGGTATTGTCTGA
GCTCCCGGCTGAGAGTGTCTGCCACCTTTTGGCAGAACCCTAGGAATCACTTCC
GCTGCCAGGTGCAGTTTTACGGCCTGAGCGAAAACGATGAATGGACACAGGAC
AGGGCCAAGCCCGTGACACAGATCGTGTCCGCCGAGGCATGGGGAAGGGCAG
ACTGTGGCTTCACCTCCGAGTCTTACCAGCAGGGCGTGCTGAGCGCCACCATC
CTGTATGAGATCCTGCTGGGCAAGGCCACACTGTACGCCGTGCTGGTGTCTGC
CCTGGTGCTGATGGCCATGGTGAAGCGGAAGGATTCCAGAGGCCGGACCGATG
GA
JFC042: miTCR2_α_S141PD_β_S32K_S33F (SEQ ID NO: 14)
gtcgacgttaacgccgccaccATGGAGACCCTGCTGGGCCTGCTGATCCTGTGGCTGCAG
CTGCAGTGGGTGAGCTCCAAGCAGGAGGTGAAGCTGCAGGAGAGCGGCCCCG
GACTGGTGGCACCTTCTCAGAGCCTGTCCGTGACCTGCACAGTGAGCGGCGTG
TCCCTGCCTGACTACGGCGTGAGCTGGATCAGGCAGCCACCTAGAAAGGGACT
GGAGTGGCTGGGCGTGATCTGGGGCTCCGAGACCACATACTATAACTCCGCCC
TGAAGTCTCGGCTGACCATCATCAAGGATAACTCTAAGAGCCAGGTGTTTCTGAA
GATGAATTCCCTGCAGACCGACGATACAGCCATCTACTATTGTGCCAAGCACTA
CTATTACGGCGGCTCTTATGCCATGGACTACTGGGGCCAGGGCACCAGCGTGA
CAGTGTCTCCCGACATCCAGAACCCTGATCCAGCCGTGTATCAGCTGAGGGACT
CCAAGTCCTCTGATAAGAGCGTGTGCCTGTTCACCGACTTTGATTCTCAGACAAA
CGTGAGCCAGTCTAAGGACAGCGACGTGTACATCACCGACAAGTGCGTGCTGG
ATATGCGCAGCATGGACTTCAAGAGCAACTCCGCCGTGGCCTGGTCTAATAAGA
GCGACTTCGCCTGCGCCAACGCCTTTAACAATTCCATCATCCCAGAGGATACATT
CTTTCCCTCTCCTGAGAGCTCCTGTGACGTGAAGCTGGTGGAGAAGAGCTTCGA
GACCGATACAAACCTGAATTTTCAGAACCTGTCCGTGATCGGCTTCCGGATCCT
GCTGCTGAAGGTGGCCGGCTTTAATCTGCTGATGACCCTGAGACTGTGGTCTAG
CAGGGCCAAGCGCGGCAGCGGCGAGGGCAGAGGAAGTCTGCTGACATGCGGA
GACGTGGAGGAGAATCCTGGACCTATGTCCATCGGACTGCTGTGCTGTGCCGC
CCTGAGCCTGCTGTGGGCCGGCCCAGTGAACGCAGGCGTGGACATCCAGATGA
CCCAGACCACAAAGTTCCTGTCTGCCAGCCTGGGCGACAGGGTGACAATCAGC
TGCCGCGCCTCCCAGGATATCTCTAAGTATCTGAATTGGTACCAGCAGAAGCCA
GATGGCACCGTGAAGCTGCTGATCTATCACACAAGCCGGCTGCACTCCGGAGT
GCCATCTAGATTTTCCGGATCTGGAAGCGGAACCGACTACAGCCTGACAATCTC
CAACCTGGAGCAGGAGGATATCGCCACCTATTTTTGCCAGCAGGGCAATACCCT
GCCCTACACATTCGGCGGCGGCACCAAGCTGGAGATCACAGACCTGAAGAACG
TGTTCCCACCCGAGGTGGCCGTGTTTGAGCCTTCTGAGGCCGAGATCAGCCAC
ACCCAGAAGGCCACCCTGGTGTGCCTGGCAACCGGCTTCTATCCAGATCACGT
GGAGCTGAGCTGGTGGGTGAACGGCAAGGAGGTGCACTCCGGCGTGTGCACA
GACCCTCAGCCACTGAAGGAGCAGCCCGCCCTGAATGATTCCCGGTATTGTCTG
AGCTCCCGGCTGAGAGTGTCTGCCACCTTTTGGCAGAACCCTAGGAATCACTTC
CGCTGCCAGGTGCAGTTTTACGGCCTGAGCGAAAACGATGAATGGACACAGGA
CAGGGCCAAGCCCGTGACACAGATCGTGTCCGCCGAGGCATGGGGAAGGGCA
GACTGTGGCTTCACCTCCGAGTCTTACCAGCAGGGCGTGCTGAGCGCCACCAT
CCTGTATGAGATCCTGCTGGGCAAGGCCACACTGTACGCCGTGCTGGTGTCTG
CCCTGGTGCTGATGGCCATGGTGAAGCGGAAGGATTCCAGAGGCCGGACCGAT
GGA
JFC043: miTCR2_α_S141PD_T108L_β_wt (SEQ ID NO: 15)
gtcgacgttaacgccgccaccATGGAGACCCTGCTGGGCCTGCTGATCCTGTGGCTGCAG
CTGCAGTGGGTGAGCTCCAAGCAGGAGGTGAAGCTGCAGGAGAGCGGCCCCG
GACTGGTGGCACCTTCTCAGAGCCTGTCCGTGACCTGCACAGTGAGCGGCGTG
TCCCTGCCTGACTACGGCGTGAGCTGGATCAGGCAGCCACCTAGAAAGGGACT
GGAGTGGCTGGGCGTGATCTGGGGCTCCGAGACCACATACTATAACTCCGCCC
TGAAGTCTCGGCTGACCATCATCAAGGATAACTCTAAGAGCCAGGTGTTTCTGAA
GATGAATTCCCTGCAGctgGACGATACAGCCATCTACTATTGTGCCAAGCACTAC
TATTACGGCGGCTCTTATGCCATGGACTACTGGGGCCAGGGCACCAGCGTGAC
AGTGTCTCCCGACATCCAGAACCCTGATCCAGCCGTGTATCAGCTGAGGGACTC
CAAGTCCTCTGATAAGAGCGTGTGCCTGTTCACCGACTTTGATTCTCAGACAAAC
GTGAGCCAGTCTAAGGACAGCGACGTGTACATCACCGACAAGTGCGTGCTGGA
TATGCGCAGCATGGACTTCAAGAGCAACTCCGCCGTGGCCTGGTCTAATAAGAG
CGACTTCGCCTGCGCCAACGCCTTTAACAATTCCATCATCCCAGAGGATACATTC
TTTCCCTCTCCTGAGAGCTCCTGTGACGTGAAGCTGGTGGAGAAGAGCTTCGAG
ACCGATACAAACCTGAATTTTCAGAACCTGTCCGTGATCGGCTTCCGGATCCTG
CTGCTGAAGGTGGCCGGCTTTAATCTGCTGATGACCCTGAGACTGTGGTCTAGC
AGGGCCAAGCGCGGCAGCGGCGAGGGCAGAGGAAGTCTGCTGACATGCGGAG
ACGTGGAGGAGAATCCTGGACCTATGTCCATCGGACTGCTGTGCTGTGCCGCC
CTGAGCCTGCTGTGGGCCGGCCCAGTGAACGCAGGCGTGGACATCCAGATGAC
CCAGACCACATCCTCTCTGTCTGCCAGCCTGGGCGACAGGGTGACAATCAGCT
GCCGCGCCTCCCAGGATATCTCTAAGTATCTGAATTGGTACCAGCAGAAGCCAG
ATGGCACCGTGAAGCTGCTGATCTATCACACAAGCCGGCTGCACTCCGGAGTG
CCATCTAGATTTTCCGGATCTGGAAGCGGAACCGACTACAGCCTGACAATCTCC
AACCTGGAGCAGGAGGATATCGCCACCTATTTTTGCCAGCAGGGCAATACCCTG
CCCTACACATTCGGCGGCGGCACCAAGCTGGAGATCACAGACCTGAAGAACGT
GTTCCCACCCGAGGTGGCCGTGTTTGAGCCTTCTGAGGCCGAGATCAGCCACA
CCCAGAAGGCCACCCTGGTGTGCCTGGCAACCGGCTTCTATCCAGATCACGTG
GAGCTGAGCTGGTGGGTGAACGGCAAGGAGGTGCACTCCGGCGTGTGCACAG
ACCCTCAGCCACTGAAGGAGCAGCCCGCCCTGAATGATTCCCGGTATTGTCTGA
GCTCCCGGCTGAGAGTGTCTGCCACCTTTTGGCAGAACCCTAGGAATCACTTCC
GCTGCCAGGTGCAGTTTTACGGCCTGAGCGAAAACGATGAATGGACACAGGAC
AGGGCCAAGCCCGTGACACAGATCGTGTCCGCCGAGGCATGGGGAAGGGCAG
ACTGTGGCTTCACCTCCGAGTCTTACCAGCAGGGCGTGCTGAGCGCCACCATC
CTGTATGAGATCCTGCTGGGCAAGGCCACACTGTACGCCGTGCTGGTGTCTGC
CCTGGTGCTGATGGCCATGGTGAAGCGGAAGGATTCCAGAGGCCGGACCG
JFC044: miTCR2_α_S141PD_T108L_β_S32K_S33F (SEQ ID NO: 16)
gtcgacgttaacgccgccaccATGGAGACCCTGCTGGGCCTGCTGATCCTGTGGCTGCAG
CTGCAGTGGGTGAGCTCCAAGCAGGAGGTGAAGCTGCAGGAGAGCGGCCCCG
GACTGGTGGCACCTTCTCAGAGCCTGTCCGTGACCTGCACAGTGAGCGGCGTG
TCCCTGCCTGACTACGGCGTGAGCTGGATCAGGCAGCCACCTAGAAAGGGACT
GGAGTGGCTGGGCGTGATCTGGGGCTCCGAGACCACATACTATAACTCCGCCC
TGAAGTCTCGGCTGACCATCATCAAGGATAACTCTAAGAGCCAGGTGTTTCTGAA
GATGAATTCCCTGCAGctgGACGATACAGCCATCTACTATTGTGCCAAGCACTAC
TATTACGGCGGCTCTTATGCCATGGACTACTGGGGCCAGGGCACCAGCGTGAC
AGTGTCTCCCGACATCCAGAACCCTGATCCAGCCGTGTATCAGCTGAGGGACTC
CAAGTCCTCTGATAAGAGCGTGTGCCTGTTCACCGACTTTGATTCTCAGACAAAC
GTGAGCCAGTCTAAGGACAGCGACGTGTACATCACCGACAAGTGCGTGCTGGA
TATGCGCAGCATGGACTTCAAGAGCAACTCCGCCGTGGCCTGGTCTAATAAGAG
CGACTTCGCCTGCGCCAACGCCTTTAACAATTCCATCATCCCAGAGGATACATTC
TTTCCCTCTCCTGAGAGCTCCTGTGACGTGAAGCTGGTGGAGAAGAGCTTCGAG
ACCGATACAAACCTGAATTTTCAGAACCTGTCCGTGATCGGCTTCCGGATCCTG
CTGCTGAAGGTGGCCGGCTTTAATCTGCTGATGACCCTGAGACTGTGGTCTAGC
AGGGCCAAGCGCGGCAGCGGCGAGGGCAGAGGAAGTCTGCTGACATGCGGAG
ACGTGGAGGAGAATCCTGGACCTATGTCCATCGGACTGCTGTGCTGTGCCGCC
CTGAGCCTGCTGTGGGCCGGCCCAGTGAACGCAGGCGTGGACATCCAGATGAC
CCAGACCACAaagTTCCTGTCTGCCAGCCTGGGCGACAGGGTGACAATCAGCTG
CCGCGCCTCCCAGGATATCTCTAAGTATCTGAATTGGTACCAGCAGAAGCCAGA
TGGCACCGTGAAGCTGCTGATCTATCACACAAGCCGGCTGCACTCCGGAGTGC
CATCTAGATTTTCCGGATCTGGAAGCGGAACCGACTACAGCCTGACAATCTCCA
ACCTGGAGCAGGAGGATATCGCCACCTATTTTTGCCAGCAGGGCAATACCCTGC
CCTACACATTCGGCGGCGGCACCAAGCTGGAGATCACAGACCTGAAGAACGTG
TTCCCACCCGAGGTGGCCGTGTTTGAGCCTTCTGAGGCCGAGATCAGCCACAC
CCAGAAGGCCACCCTGGTGTGCCTGGCAACCGGCTTCTATCCAGATCACGTGG
AGCTGAGCTGGTGGGTGAACGGCAAGGAGGTGCACTCCGGCGTGTGCACAGAC
CCTCAGCCACTGAAGGAGCAGCCCGCCCTGAATGATTCCCGGTATTGTCTGAGC
TCCCGGCTGAGAGTGTCTGCCACCTTTTGGCAGAACCCTAGGAATCACTTCCGC
TGCCAGGTGCAGTTTTACGGCCTGAGCGAAAACGATGAATGGACACAGGACAG
GGCCAAGCCCGTGACACAGATCGTGTCCGCCGAGGCATGGGGAAGGGCAGAC
TGTGGCTTCACCTCCGAGTCTTACCAGCAGGGCGTGCTGAGCGCCACCATCCT
GTATGAGATCCTGCTGGGCAAGGCCACACTGTACGCCGTGCTGGTGTCTGCCC
TGGTGCTGATGGCCATGGTGAAGCGGAAGGATTCCAGAGGCCGGACCGA

The DNA sequences encoding the miTCRs can include Kozak DNA sequences (gcegccacc) in front of the protein start codon (M) to boost the protein expression. It is part of the design and not in the commercial plasmid backbone. The taagaggccgc DNA trails the sequence. They both are DNA sequences flanking the DNA sequences coding the protein. They are not translated into the protein and can be removed without effects on protein sequences.

TABLE 4 describes protein sequences for various miTCR constructs. FMC63Vl is the variable light chain from antibody FMC63 that recognizes CD19. FMC63Vh is the variable heavy chain from antibody FMC63 that recognizes CD19. TCRalpha is the TCR alpha constant region. TCRbeta is the TCR p constant region. mutC refers to one amino acid in the constant chain that has been mutated to cysteine to promote disulfide bond formation. Furin refers to a Furin recognition site that was introduced to shorten the tails of the protein after the 2A cleavage. Mutation or insertion is depicted by enlarged and double-underlined text. The sequence with wavy underline is P2A, enlarged and bolded is mCherry (marker protein).

TABLE 4
Protein (amino acid) sequences of miTCR1 and miTCR2
constructs. Parent constructs are indicated by dotted or dashed
underlines. Furin sequences are indicated by enlarged and
italicized text. Modifications to the parent constructs are
depicted by enlarged and double-underlined text.

JFC029: miTCR1_α_wt_β_wt (SEQ ID NO: 17)

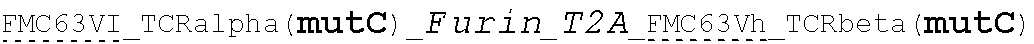

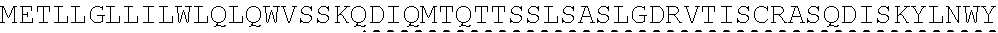

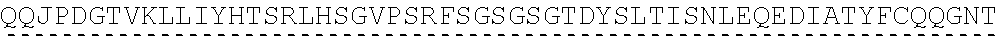

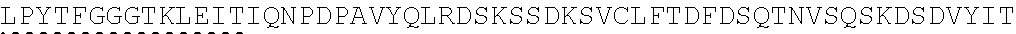

DK [symbol] VLDMRSMDFKSNSAVAWSNKSDFACANAFNNSIIPEDTFFPSPESSCDVKLVE
KSFETDTNLNFQNLSVIGFRILLLKVAGFNLLMTLRLWSS

[symbol]

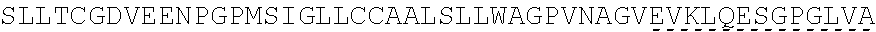

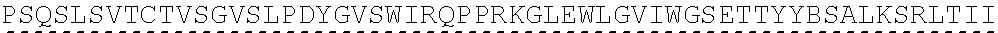

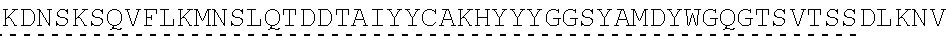

FPPEVAVFEPSEAEISHTQKATLVCLATGFYPDHVELSWWVNGKEVHSGV [symbol] TDPQ
PLKEQPALNDSRYCLSSRLRVSATFWQNPRNHFRCQVQFYGLSENDEWTQDRAKP
VTQIVSAEAWGRADCGFTSESYQQGVLSATILYEILLGKATLYAVLVSALVLMAMVKR
KDSRGRTDG*
JFC039: miTCR2_α_wt_β_wt (SEQ ID NO: 18)

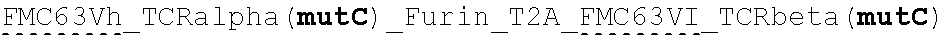

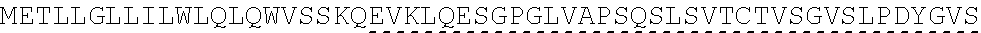

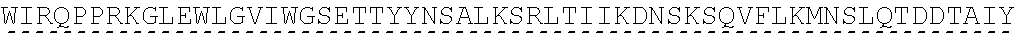

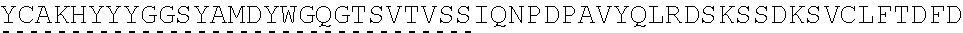

SQTNVSQSKDSDVYITDK [symbol] VLDMRSMDFKSNSAVAWSNKSDFACANAFNNSIIPED
TFFPSPESSCDVKLVEKSFETDTNLNFQNLSVIGFRILLLKVAGFNLLMTLRLWSS [symbol] [symbol] [symbol]
MSIGLLCCAALSLLWAGPVN

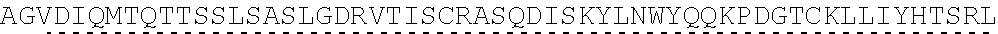

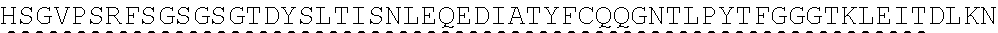

VFPPEVAVFEPSEAEISHTQKATLVCLATGFYPDHVELSWWVNGKEVHSGV [symbol] TDP
QPLKEQPALNDSRYCLSSRLRVSATFWQNPRNHFRCQVQFYGLSENDEWTQDRA
KPVTQIVSAEAWGRADCGFTSESYQQGVLSATILYEILLGKATLYAVLVSALVLMAMV
KRKDSRGRTDG*
JFC030: miTCR1_α_wt_β_G31K_P32F (SEQ ID NO: 19)

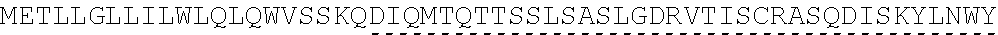

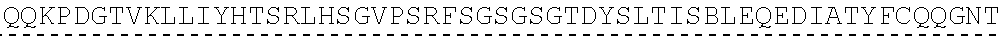

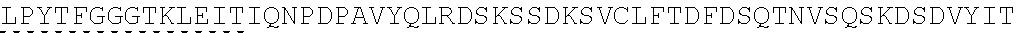

DK [symbol] VLDMRSMDFKSNSAVAWSNKSDFACANAFNNSIIPEDTFFPSPESSCDVKLVE
KSFETDTNLNFQNLSVIGFRILLLKVAGFNLLMTLRLWSS

[symbol]

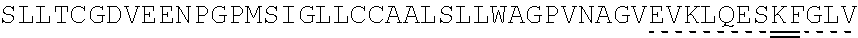

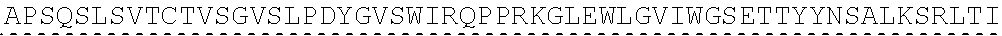

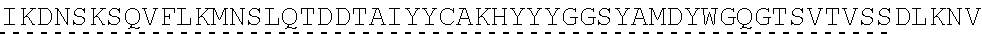

FPPEVAVFEPSEAEISHTQKATLVCLATGFYPDHVELSWWVNGKEVHSGV [symbol] TDPQ
PLKEQPALNDSRYCLSSRLRVSATFWQNPRNHFRCQVQFYGLSENDEWTQDRAKP
VTQIVSAEAWGRADCGFTSESYQQGVLSATILYEILLGKATLYAVLVSALVLMAMVKR

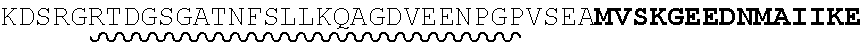

FMRFKVHMEGSVNGHEFEIEGEGEGRPYEGTQTAKLK
VTKGGPLPFAWDILSPQFMYGSKAYVKHPADIPDYLKL
SFPEGFKWERVMNFEDGGVVTVTQDSSLQDGEFIYKVK
LRGTNFPSDGPVMQKKTMGWEASSERMYPEDGALKG
EIKQRLKLKDGGHYDAEVKTTYKAKKPVQLPGAYNVNI
KLDITSHNEDYTIVEQYERAEGRHSTGGMDELYK
JFC031: miTCR1_α_PD_B_wt (SEQ ID NO: 20)

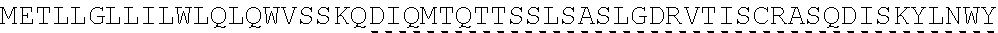

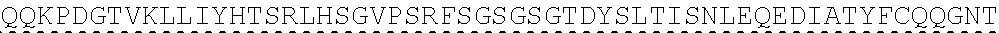

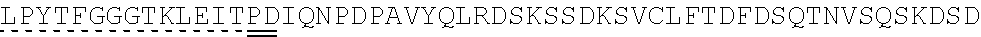

VYITDK [symbol] VLDMRSMDFKSNSAVAWSNKSDFACANAFNNSIIPEDTFFPSPESSCDV
KLVEKSFETDTNLNFQNLSVIGFRILLLKVAGFNLLMTLRLWSS

[symbol]

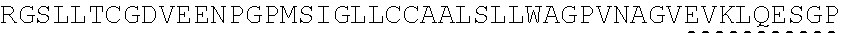

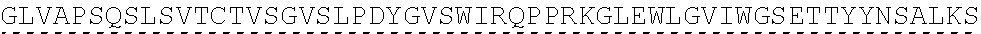

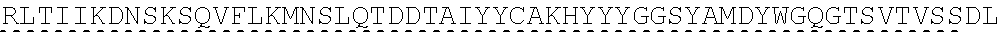

KNVFPPEVAVFEPSEAEISHTQKATLVCLATGFYPDHVELSWWVNGKEVHSGV [symbol] CT
DPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMA

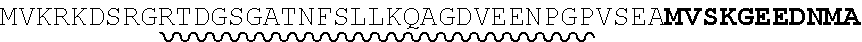

IIKEFMRFKVHMEGSVNGHEFEIEGEGEGRPYEGTQTA
KLKVTKGGPLPFAWDILSPQFMYGSKAYVKHPADIPDY
LKLSFPEGFKWERVMNFEDGGVVTVTQDSSLQDGEFIY
KVKLRGTNFPSDGPVMQKKTMGWEASSERMYPEDGA
LKGEIKQRLKLKDGGHYDAEVKTTYKAKKPVQLPGAY
NVNIKLDITSHNEDYTIVEQYERAEGRHSTGGMDELYK
JFC032: miTCR1_α_PD_β_G31K_P32F (SEQ ID NO: 21)

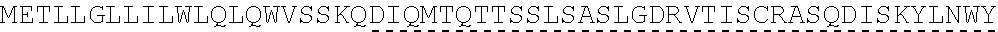

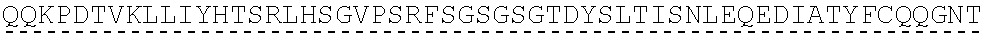

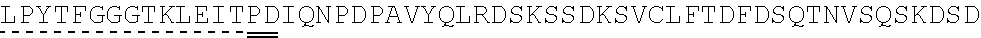

VYITDK [symbol] VLDMRSMDFKSNSAVAWSNKSDFACANAFNNSIIPEDTFFPSPESSCDV
KLVEKSFETDTNLNFQNLSVIGFRILLLKVAGFNLLMTLRLWSS

[symbol]

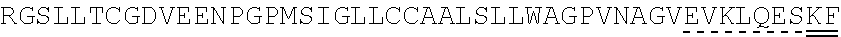

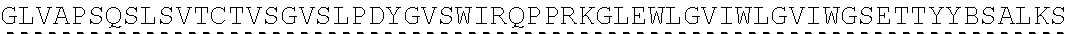

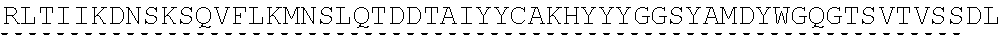

KNVFPPEVAVFEPSEAEISHTQKATLVCLATGFYPDHVELSWWVNGKEVHSGV [symbol] T
DPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMA

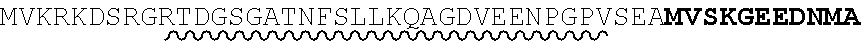

IIKEFMRFKVHMEGSVNGHEFEIEGEGEGRPYEGTQTA
KLKVTKGGPLPFAWDILSPQFMYGSKAYVKHPADIPDY
LKLSFPEGFKWERVMNFEDGGVVTVTQDSSLQDGEFIY
KVKLRGTNFPSDGPVMQKKTMGWEASSERMYPEDGA
LKGEIKQRLKLKDGGHYDAEVKTTYKAKKPVQLPGAY
NVNIKLDITSHNEDYTIVEQYERAEGRHSTGGMDELYK
JFC033: miTCR1_α_Q101L_β_wt (SEQ ID NO: 22)

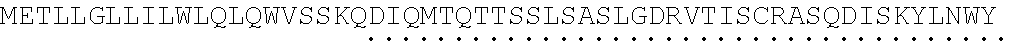

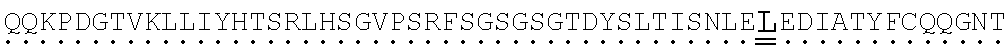

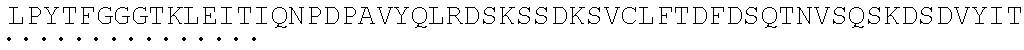

DK [symbol] VLDMRSMDFKSNSAVAWSNKSDFACANAFNNSIIPEDTFFPSPESSCDVKLVE
KSFETDTNLNFQNLSVIGFRILLLKVAGFNLLMTLRLWSS

[symbol]

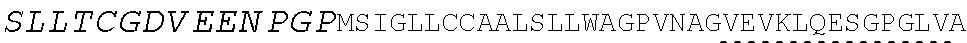

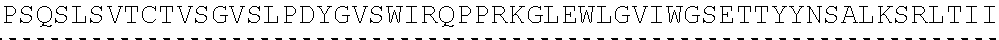

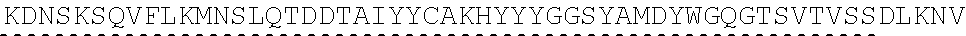

FPPEVAVFEPSEAEISHTQKATLVCLATGFYPDHVELSWWVNGKEVHSGV [symbol] TDPQ
PLKEQPALNDSRYCLSSRLRVSATFWQNPRNHFRCQVQFYGLSENDEWTQDRAKP
VTQIVSAEAWGRADCGFTSESYQQGVLSATILYEILLGKATLYAVLVSALVLMAMVKR

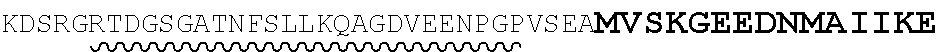

FMRFKVHMEGSVNGHEFEIEGEGEGRPYEGTQTAKLK
VTKGGPLPFAWDILSPQFMYGSKAYVKHPADIPDYLKL
SFPEGFKWERVMNFEDGGVVTVTQDSSLQDGEFIYKVK
LRGTNFPSDGPVMQKKTMGWEASSERMYPEDGALKG
EIKQRLKLKDGGHYDAEVKTTYKAKKPVQLPGAYNVNI
KLDITSHNEDYTIVEQYERAEGRHSTGGMDELYK
JFC034: miTCR1_α_PD_Q101L_β_wt (SEQ ID NO: 23)

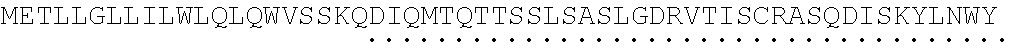

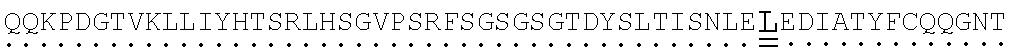

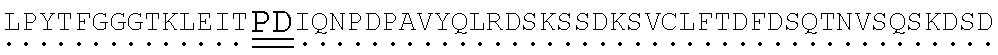

VYITDK [symbol] VLDMRSMDFKSNSAVAWSNKSDFACANAFNNSIIPEDTFFPSPESSCDV
KLVEKSFETDTNLNFQNLSVIGFRILLLKVAGFNLLMTLRLWSS

[symbol]

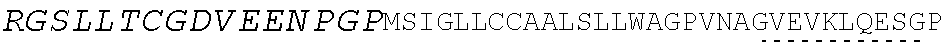

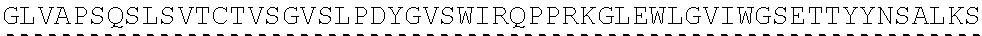

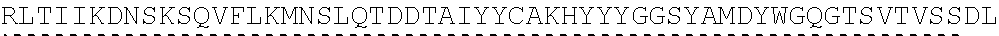

KNVFPPEVAVFEPSEAEISHTQKATLVCLATGFYPDHVELSWWVNGKEVHSGV [symbol] T
DPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMA

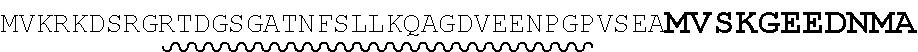

IIKEFMRFKVHMEGSVNGHEFEIEGEGEGRPYEGTQTA
KLKVTKGGPLPFAWDILSPQFMYGSKAYVKHPADIPDY
LKLSFPEGFKWERVMNFEDGGVVTVTQDSSLQDGEFIY
KVKLRGTNFPSDGPVMQKKTMGWEASSERMYPEDGA
LKGEIKQRLKLKDGGHYDAEVKTTYKAKKPVQLPGAY
NVNIKLDITSHNEDYTIVEQYERAEGRHSTGGMDELYK
JFC035: miTCR1_α_PD_Q101L_L36E_β_wt (SEQ ID NO: 24)

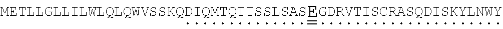

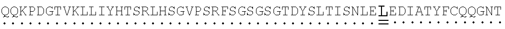

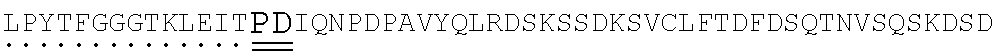

VYITDK [symbol] VLDMRSMDFKSNSAVAWSNKSDFACANAFNNSIIPEDTFFPSPESSCDV
KLVEKSFETDTNLNFQNLSVIGFRILLLKVAGFNLLMTLRLWSS

[symbol]

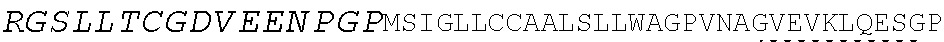

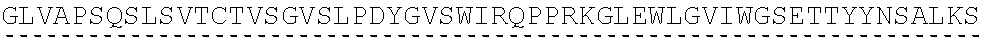

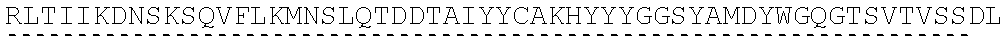

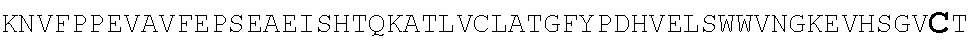

DPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMA

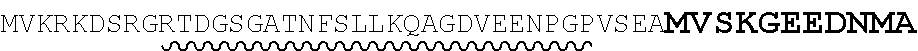

IIKEFMRFKVHMEGSVNGHEFEIEGEGEGRPYEGTQTA
KLKVTKGGPLPFAWDILSPQFMYGSKAYVKHPADIPDY
LKLSFPEGFKWERVMNFEDGGVVTVTQDSSLQDGEFIY
KVKLRGTNFPSDGPVMQKKTMGWEASSERMYPEDGA
LKGEIKQRLKLKDGGHYDAEVKTTYKAKKPVQLPGAY
NVNIKLDITSHNEDYTIVEQYERAEGRHSTGGMDELYK
JFC036: miTCR1_α_Q101L_β_G31K_P32F (SEQ ID NO: 25)

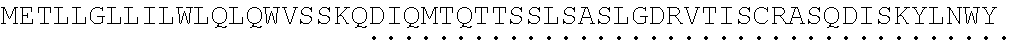

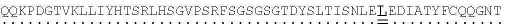

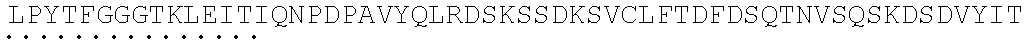

DK [symbol] VLDMRSMDFKSNSAVAWSNKSDFACANAFNNSIIPEDTFFPSPESSCDVKLVE
KSFETDTNLNFQNLSVIGFRILLLKVAGFNLLMTLRLWSS

[symbol]

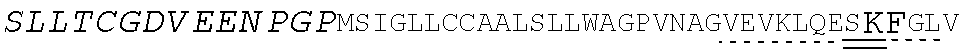

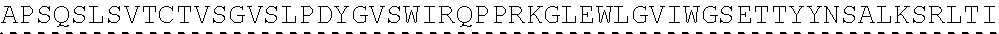

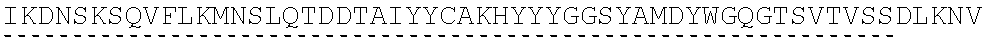

FPPEVAVFEPSEAEISHTQKATLVCLATGFYPDHVELSWWVNGKEVHSGV [symbol] TDPQ
PLKEQPALNDSRYCLSSRLRVSATFWQNPRNHFRCQVQFYGLSENDEWTQDRAKP
VTQIVSAEAWGRADCGFTSESYQQGVLSATILYEILLGKATLYAVLVSALVLMAMVKR

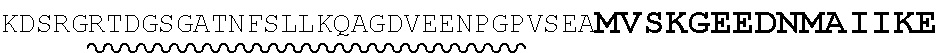

FMRFKVHMEGSVNGHEFEIEGEGEGRPYEGTQTAKLK
VTKGGPLPFAWDILSPQFMYGSKAYVKHPADIPDYLKL
SFPEGFKWERVMNFEDGGVVTVTQDSSLQDGEFIYKVK
LRGTNFPSDGPVMQKKTMGWEASSERMYPEDGALKG
EIKQRLKLKDGGHYDAEVKTTYKAKKPVQLPGAYNVNI
KLDITSHNEDYTIVEQYERAEGRHSTGGMDELYK
JFC037: miTCR1_α_PD_Q101L_β_G31K_P32F (SEQ ID NO: 26)

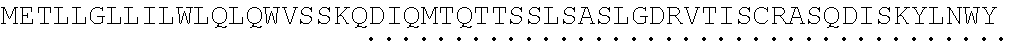

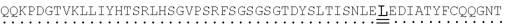

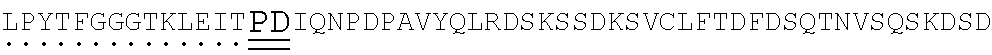

VYITDK [symbol] VLDMRSMDFKSNSAVAWSNKSDFACANAFNNSIIPEDTFFPSPESSCDV
KLVEKSFETDTNLNFQNLSVIGFRILLLKVAGFNLLMTLRLWSS

[symbol]

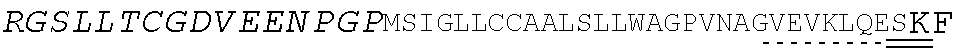

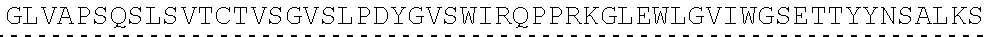

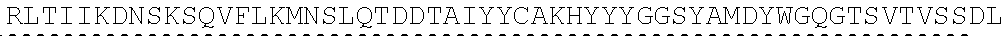

KNVFPPEVAVFEPSEAEISHTQKATLVCLATGFYPDHVELSWWVNGKEVHSGV [symbol] T
DPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMA

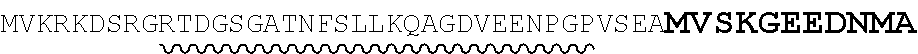

IIKEFMRFKVHMEGSVNGHEFEIEGEGEGRPYEGTQTA
KLKVTKGGPLPFAWDILSPQFMYGSKAYVKHPADIPDY
LKLSFPEGFKWERVMNFEDGGVVTVTQDSSLQDGEFIY
KVKLRGTNFPSDGPVMQKKTMGWEASSERMYPEDGA
LKGEIKQRLKLKDGGHYDAEVKTTYKAKKPVQLPGAY
NVNIKLDITSHNEDYTIVEQYERAEGRHSTGGMDELYK
JFC038: miTCR1_α_PD_Q101L_L36E_β_G31K_P32F (SEQ ID NO: 27)

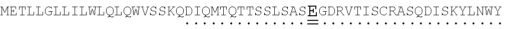

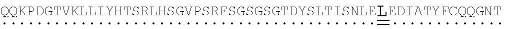

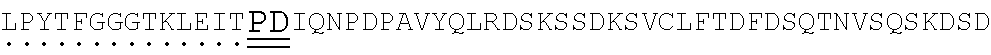

VYITDK [symbol] VLDMRSMDFKSNSAVAWSNKSDFACANAFNNSIIPEDTFFPSPESSCDV
KLVEKSFETDTNLNFQNLSVIGFRILLLKVAGFNLLMTLRLWSS

[symbol]

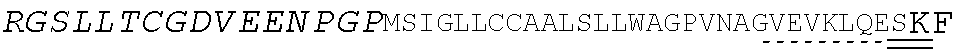

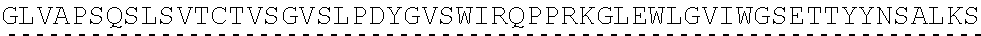

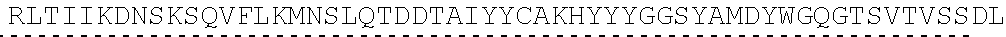

KNVFPPEVAVFEPSEAEISHTQKATLVCLATGFYPDHVELSWWVNGKEVHSGV [symbol] T
DPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLMA

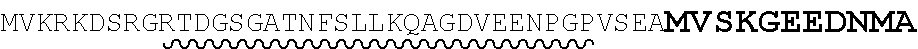

IIKEFMRFKVHMEGSVNGHEFEIEGEGEGRPYEGTQTA
KLKVTKGGPLPFAWDILSPQFMYGSKAYVKHPADIPDY
LKLSFPEGFKWERVMNFEDGGVVTVTQDSSLQDGEFIY
KVKLRGTNFPSDGPVMQKKTMGWEASSERMYPEDGA
LKGEIKQRLKLKDGGHYDAEVKTTYKAKKPVQLPGAY
NVNIKLDITSHNEDYTIVEQYERAEGRHSTGGMDELYK
JFC040: miTCR2_α_wt_β_S32K_S33F (SEQ ID NO: 28)

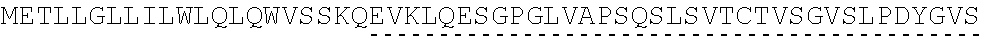

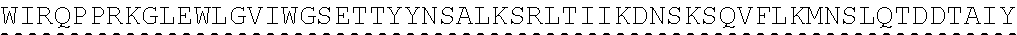

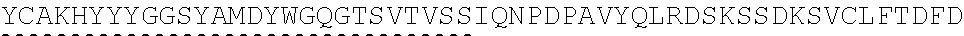

SQTNVSQSKDSDVYITDK [symbol] VLDMRSMDFKSNSAVAWSNKSDFACANAFNNSIIPED
TFFPSPESSCDVKLVEKSFETDTNLNFQNLSVIGFRILLLKVAGFNLLMTLRLWSS [symbol] [symbol] [symbol]
MSIGLLCCAALSLLWAGPVN

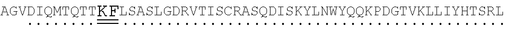

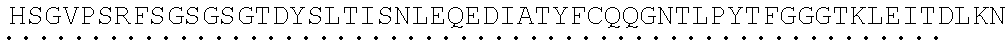

VFPPEVAVFEPSEAEISHTQKATLVCLATGFYPDHVELSWWVNGKEVHSGV [symbol] TDP
QPLKEQPALNDSRYCLSSRLRVSATFWQNPRNHFRCQVQFYGLSENDEWTQDRA
KPVTQIVSAEAWGRADCGFTSESYQQGVLSATILYEILLGKATLYAVLVSALVLMAMV

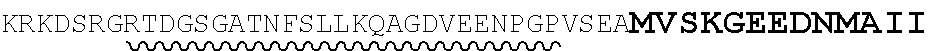

KEFMRFKVHMEGSVNGHEFEIEGEGEGRPYEGTQTAK
LKVTKGGPLPFAWDILSPQFMYGSKAYVKHPADIPDYL
KLSFPEGFKWERVMNFEDGGVVTVTQDSSLQDGEFIYK
VKLRGTNFPSDGPVMQKKTMGWEASSERMYPEDGAL
KGEIKQRLKLKDGGHYDAEVKTTYKAKKPVQLPGAYN
VNIKLDITSHNEDYTIVEQYERAEGRHSTGGMDELYK
JFC041: miTCR2_α_S141PD_β_wt (SEQ ID NO: 29)

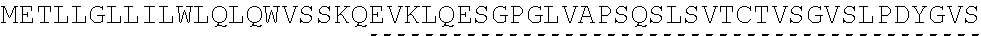

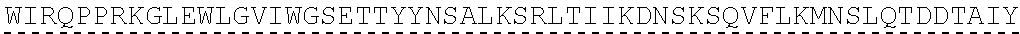

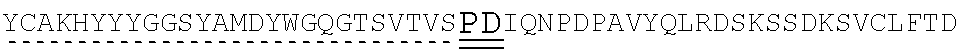

FDSQTNVSQSKDSDVYITDK [symbol] VLDMRSMDFKSNSAVAWSNKSDFACANAFNNSIIP
EDTFFPSPESSCDVKLVEKSFETDTNLNFQNLSVIGFRILLLKVAGFNLLMTLRLWSS [symbol] [symbol]
MSIGLLCCAALSLLWAGPV

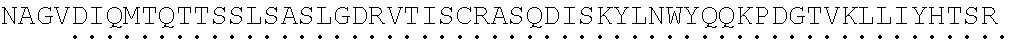

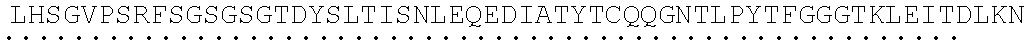

VFPPEVAVFEPSEAEISHTQKATLVCLATGFYPDHVELSWWVNGKEVHSGV [symbol] TDP
QPLKEQPALNDSRYCLSSRLRVSATFWQNPRNHFRCQVQFYGLSENDEWTQDRA
KPVTQIVSAEAWGRADCGFTSESYQQGVLSATILYEILLGKATLYAVLVSALVLMAMV

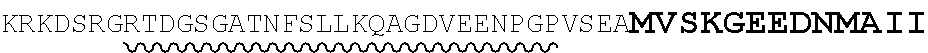

KEFMRFKVHMEGSVNGHEFEIEGEGEGRPYEGTQTAK
LKVTKGGPLPFAWDILSPQFMYGSKAYVKHPADIPDYL
KLSFPEGFKWERVMNFEDGGVVTVTQDSSLQDGEFIYK
VKLRGTNFPSDGPVMQKKTMGWEASSERMYPEDGAL
KGEIKQRLKLKDGGHYDAEVKTTYKAKKPVQLPGAYN
VNIKLDITSHNEDYTIVEQYERAEGRHSTGGMDELYK
JFC042: miTCR2_α_S141PD_β_S32K_S33F (SEQ ID NO: 30)

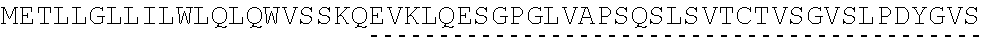

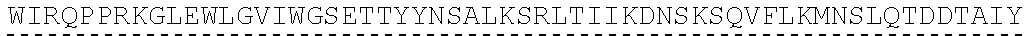

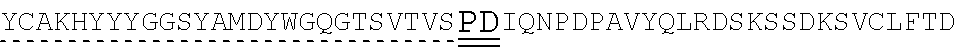

FDSQTNVSQSKDSDVYITDK [symbol] VLDMRSMDFKSNSAVAWSNKSDFACANAFNNSIIP
EDTFFPSPESSCDVKLVEKSFETDTNLNFQNLSVIGFRILLLKVAGFNLLMTLRLWSS

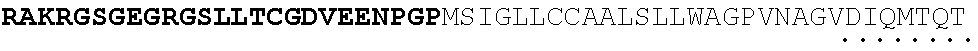

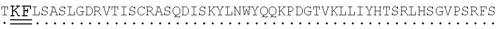

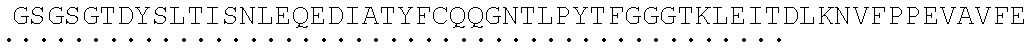

PSEAEISHTQKATLVCLATGFYPDHVELSWWVNGKEVHSGV [symbol] TDPQPLKEQPALN
DSRYCLSSRLRVSATFWQNPRNHFRCQVQFYGLSENDEWTQDRAKPVTQIVSAEA
WGRADCGFTSESYQQGVLSATILYEILLGKATLYAVLVSALVLMAMVKRKDSRGRTD

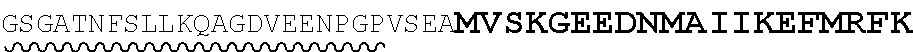

VHMEGSVNGHEFEIEGEGEGRPYEGTQTAKLKVTKGG
PLPFAWDILSPQFMYGSKAYVKHPADIPDYLKLSFPEGF
KWERVMNFEDGGVVTVTQDSSLQDGEFIYKVKLRGTN
FPSDGPVMQKKTMGWEASSERMYPEDGALKGEIKQRL
KLKDGGHYDAEVKTTYKAKKPVQLPGAYNVNIKLDITS
HNEDYTIVEQYERAEGRHSTGGMDELYK
JFC043: miTCR2_α_S141PD_T108L_β_wt (SEQ ID NO: 31)

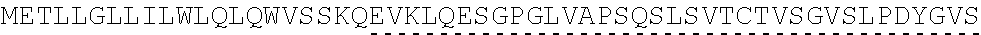

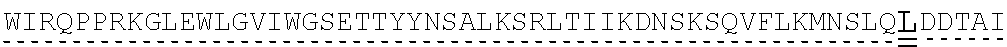

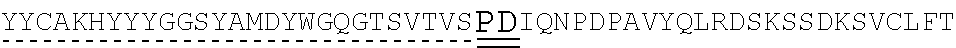

DFDSQTNVSQSKDSDVYITDK [symbol] VLDMRSMDFKSNSAVAWSNKSDFACANAFNNSII
PEDTFFPSPESSCDVKLVEKSFETDTNLNFQNLSVIGFRILLLKVAGFNLLMTLRLWS
s [symbol] [symbol] MSIGLLCCAALSLLWAGP

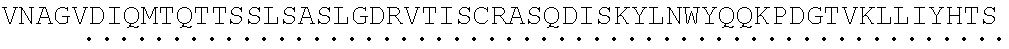

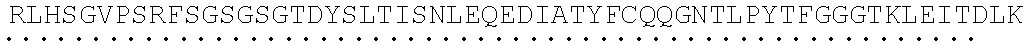

NVFPPEVAVFEPSEAEISHTQKATLVCLATGFYPDHVELSWWVNGKEVHSGV [symbol] TD
PQPLKEQPALNDSRYCLSSRLRVSATFWQNPRNHFRCQVQFYGLSENDEWTQDR
AKPVTQIVSAEAWGRADCGFTSESYQQGVLSATILYEILLGKATLYAVLVSALVLMAM

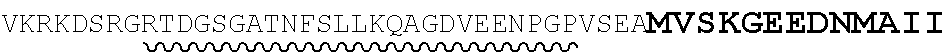

KEFMRFKVHMEGSVNGHEFEIEGEGEGRPYEGTQTAK
LKVTKGGPLPFAWDILSPQFMYGSKAYVKHPADIPDYL
KLSFPEGFKWERVMNFEDGGVVTVTQDSSLQDGEFIYK
VKLRGTNFPSDGPVMQKKTMGWEASSERMYPEDGAL
KGEIKQRLKLKDGGHYDAEVKTTYKAKKPVQLPGAYN
VNIKLDITSHNEDYTIVEQYERAEGRHSTGGMDELYK
JFC044: miTCR2_α_S141PD_T108L_β_S32K_S33F (SEQ ID NO: 32)

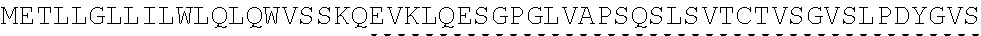

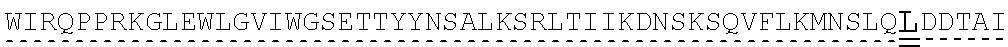

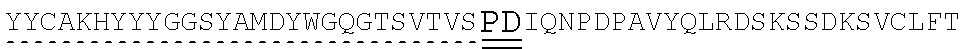

DFDSQTNVSQSKDSDVYITDK [symbol] VLDMRSMDFKSNSAVAWSNKSDFACANAFNNSII
PEDTFFPSPESSCDVKLVEKSFETDTNLNFQNLSVIGFRILLLKVAGFNLLMTLRLWS
s [symbol] [symbol] MSIGLLCCAALSLLWAGP

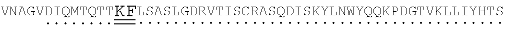

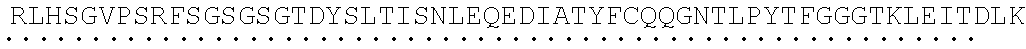

NVFPPEVAVFEPSEAEISHTQKATLVCLATGFYPDHVELSWWVNGKEVHSGV [symbol] TD
PQPLKEQPALNDSRYCLSSRLRVSATFWQNPRNHFRCQVQFYGLSENDEWTQDR
AKPVTQIVSAEAWGRADCGFTSESYQQGVLSATILYEILLGKATLYAVLVSALVLMAM

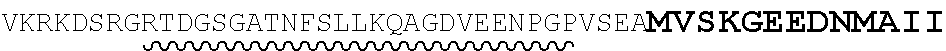

KEFMRFKVHMEGSVNGHEFEIEGEGEGRPYEGTQTAK
LKVTKGGPLPFAWDILSPQFMYGSKAYVKHPADIPDYL
KLSFPEGFKWERVMNFEDGGVVTVTQDSSLQDGEFIYK
VKLRGTNFPSDGPVMQKKTMGWEASSERMYPEDGAL
KGEIKQRLKLKDGGHYDAEVKTTYKAKKPVQLPGAYN
VNIKLDITSHNEDYTIVEQYERAEGRHSTGGMDELYK

TABLE 5
Protein sequence for native or wild type TCR constant regions
with the C mutation.

Wild type TCR alpha constant chain with T47C mutation (SEQ ID NO: 33):
IQNPDPAVYQLRDSKSSDKSVCLFTDFDSQTNVSQSKDSDVYITDKCVLDM
RSMDFKSNSAVAWSNKSDFACANAFNNSIIPEDTFFPSPESSCDVKLVEKSF
ETDTNLNFQNLSVIGFRILLLKVAGFNLLMTLRLWSS
Wild type TCR beta constant chain with S56C (SEQ ID NO: 34):
DLKNVFPPEVAVFEPSEAEISHTQKATLVCLATGFYPDHVELSWWVNGKEVH
SGVCTDPQPLKEQPALNDSRYCLSSRLRVSATFWQNPRNHFRCQVQFYGL
SENDEWTQDRAKPVTQIVSAEAWGRADCGFTSESYQQGVLSATILYEILLGK
ATLYAVLVSALVLMAMVKRKDSRG

TABLE 6 shows the reference sequence, SEQ ID NO: 35. All the mutation in VL is based on SEQ ID NO: 35. For example, L36E (the residue enlarged and double-underlined in the protein sequence in TABLE 4), is at position 36 counted from the most left M, E, T, L, L, G, L, et al., the position 36 of the wild type sequence is L (enlarged and bolded here), but mutated into E. However, for the PD mutation, the PD is an insertion, so it does not have a numbering associated with it.

TABLE 6
Protein sequence for wild type V_{L} and V_{H}.

Wild type VL (SEQ ID NO: 35)
METLLGLLILWLQLQWVSSKQDIQMTQTTSSLSASLGDRVTISCRASQD
ISKYLNWYQQKPDGTVKLLIYHTSRLHSGVPSRFSGSGSGTDYSLTISN
LEQEDIATYFCQQGNTLPYTFGGGTKLEIT
Wild type VH (SEQ ID NO: 36)
MSIGLLCCAALSLLWAGPVNAGVEVKLQESGPGLVAPSQSLSVTCTVSG
VSLPDYGVSWIRQPPRKGLEWLGVIWGSETTYYNSALKSRLTIIKDNSK
SQVFLKMNSLQTDDTAIYYCAKHYYYGGSYAMDYWGQGTSVTVSS

The point mutations are based on the wild type sequence. See TABLE 7 for residues for the wild type. All mutations are point mutations (except for the PD insertion), so in these mutated residues, the beginning and the end sequences are the same as wild type (except PD).

TABLE 7
DNA sequences for miTCR constant and variable
regions corresponding to miTCR1 and miTCR2.

MiTCR1_VL (SEQ ID NO: 37):
ATGGAGACCCTGCTGGGCCTGCTGATCCTGTGGCTGCAGCTGCAGTGG
GTGAGCTCCAAGCAGGACATCCAGATGACCCAGACCACATCTAGCCTGT
CTGCCAGCCTGGGCGATAGGGTGACAATCTCCTGCCGCGCCTCTCAGGA
CATCAGCAAGTATCTGAACTGGTACCAGCAGAAGCCAGATGGCACCGTG
AAGCTGCTGATCTATCACACATCCCGGCTGCACTCTGGCGTGCCCAGCA
GATTTTCCGGATCTGGAAGCGGAACCGACTACTCCCTGACAATCTCTAAC
CTGGAGCAGGAGGATATCGCCACCTATTTTTGTCAGCAGGGCAATACCCT
GCCTTACACATTCGGCGGCGGCACCAAGCTGGAGATCACA
MiTCR1_VH (SEQ ID NO: 38):
ATGTCCATCGGACTGCTGTGCTGTGCCGCCCTGAGCCTGCTGTGGGCCG
GCCCAGTGAACGCAGGAGTGGAGGTGAAGCTGCAGGAGTCCGGCCCCG
GACTGGTGGCACCTTCTCAGAGCCTGTCCGTGACCTGCACAGTGTCTGG
CGTGAGCCTGCCAGACTACGGCGTGAGCTGGATCAGGCAGCCACCTAG
AAAGGGACTGGAGTGGCTGGGCGTGATCTGGGGCTCCGAGACCACATA
CTATAATAGCGCCCTGAAGTCCCGGCTGACCATCATCAAGGATAACTCTA
AGAGCCAGGTGTTTCTGAAGATGAATAGCCTGCAGACCGACGATACAGC
CATCTACTATTGCGCCAAGCACTACTATTACGGCGGCTCTTATGCCATGG
ACTACTGGGGCCAGGGCACCAGCGTGACAGTGTCCTCT
MiTCR1_Ca (SEQ ID NO: 39):
ATCCAGAACCCTGACCCAGCCGTGTATCAGCTGAGGGACTCCAAGTCCT
CTGATAAGAGCGTGTGCCTGTTCACCGACTTTGATTCTCAGACAAACGTG
AGCCAGTCTAAGGACAGCGACGTGTACATCACCGACAAGTGCGTGCTGG
ATATGCGCAGCATGGACTTCAAGAGCAACTCCGCCGTGGCCTGGAGCAA
TAAGTCCGATTTCGCCTGCGCCAACGCCTTTAACAATTCCATCATCCCAG
AGGATACATTCTTTCCCTCTCCTGAGAGCTCCTGTGACGTGAAGCTGGTG
GAGAAGAGCTTCGAGACCGATACAAACCTGAATTTTCAGAACCTGTCCGT
GATCGGCTTCCGGATCCTGCTGCTGAAGGTGGCCGGCTTTAATCTGCTG
ATGACCCTGAGACTGTGGTCTAGC
MiTCR1_Cb (SEQ ID NO: 40):
GATCTGAAGAACGTGTTCCCACCCGAGGTGGCCGTGTTTGAGCCTTCTG
AGGCCGAGATCAGCCACACCCAGAAGGCCACCCTGGTGTGCCTGGCAA
CCGGCTTCTATCCAGACCACGTGGAGCTGAGCTGGTGGGTGAACGGCAA
GGAGGTGCACTCCGGCGTGTGCACAGACCCTCAGCCACTGAAGGAGCA
GCCCGCCCTGAATGATTCCCGGTATTGTCTGAGCTCCCGGCTGAGAGTG
TCTGCCACCTTTTGGCAGAACCCTAGGAATCACTTCCGCTGCCAGGTGCA
GTTTTACGGCCTGAGCGAAAAcGATGAaTGGACaCAGGACAGGGCCAAG
CCCGTGACACAGATCGTGTCCGCCGAGGCATGGGGAAGGGCAGACTGT
GGCTTCACCTCCGAGTCTTACCAGCAGGGCGTGCTGAGCGCCACCATCC
TGTATGAGATCCTGCTGGGCAAGGCCACACTGTACGCCGTGCTGGTGTC
TGCCCTGGTGCTGATGGCCATGGTGAAGCGGAAGGATTCCAGAGGC
MiTCR2_VL (SEQ ID NO: 41):
ATGTCCATCGGACTGCTGTGCTGTGCCGCCCTGAGCCTGCTGTGGGCCG
GCCCAGTGAACGCAGGCGTGGACATCCAGATGACCCAGACCACATCCTC
TCTGTCTGCCAGCCTGGGCGACAGGGTGACAATCAGCTGCCGCGCCTCC
CAGGATATCTCTAAGTATCTGAATTGGTACCAGCAGAAGCCAGATGGCAC
CGTGAAGCTGCTGATCTATCACACAAGCCGGCTGCACTCCGGAGTGCCA
TCTAGATTTTCCGGATCTGGAAGCGGAACCGACTACAGCCTGACAATCTC
CAACCTGGAGCAGGAGGATATCGCCACCTATTTTTGCCAGCAGGGCAAT
ACCCTGCCCTACACATTCGGCGGCGGCACCAAGCTGGAGATCACA
MiTCR2_VH (SEQ ID NO: 42):
ATGGAGACCCTGCTGGGCCTGCTGATCCTGTGGCTGCAGCTGCAGTGG
GTGAGCTCCAAGCAGGAGGTGAAGCTGCAGGAGAGCGGCCCCGGACTG
GTGGCACCTTCTCAGAGCCTGTCCGTGACCTGCACAGTGAGCGGCGTGT
CCCTGCCTGACTACGGCGTGAGCTGGATCAGGCAGCCACCTAGAAAGG
GACTGGAGTGGCTGGGCGTGATCTGGGGCTCCGAGACCACATACTATAA
CTCCGCCCTGAAGTCTCGGCTGACCATCATCAAGGATAACTCTAAGAGCC
AGGTGTTTCTGAAGATGAATTCCCTGCAGACCGACGATACAGCCATCTAC
TATTGTGCCAAGCACTACTATTACGGCGGCTCTTATGCCATGGACTACTG
GGGCCAGGGCACCAGCGTGACAGTGTCTAGC
MiTCR2_Ca (SEQ ID NO: 43):
ATCCAGAACCCTGATCCAGCCGTGTATCAGCTGAGGGACTCCAAGTCCT
CTGATAAGAGCGTGTGCCTGTTCACCGACTTTGATTCTCAGACAAACGTG
AGCCAGTCTAAGGACAGCGACGTGTACATCACCGACAAGTGCGTGCTGG
ATATGCGCAGCATGGACTTCAAGAGCAACTCCGCCGTGGCCTGGTCTAA
TAAGAGCGACTTCGCCTGCGCCAACGCCTTTAACAATTCCATCATCCCAG
AGGATACATTCTTTCCCTCTCCTGAGAGCTCCTGTGACGTGAAGCTGGTG
GAGAAGAGCTTCGAGACCGATACAAACCTGAATTTTCAGAACCTGTCCGT
GATCGGCTTCCGGATCCTGCTGCTGAAGGTGGCCGGCTTTAATCTGCTG
ATGACCCTGAGACTGTGGTCTAGC
MiTCR2_Cb (SEQ ID NO: 44):
GACCTGAAGAACGTGTTCCCACCCGAGGTGGCCGTGTTTGAGCCTTCTG
AGGCCGAGATCAGCCACACCCAGAAGGCCACCCTGGTGTGCCTGGCAA
CCGGCTTCTATCCAGATCACGTGGAGCTGAGCTGGTGGGTGAACGGCAA
GGAGGTGCACTCCGGCGTGTGCACAGACCCTCAGCCACTGAAGGAGCA
GCCCGCCCTGAATGATTCCCGGTATTGTCTGAGCTCCCGGCTGAGAGTG
TCTGCCACCTTTTGGCAGAACCCTAGGAATCACTTCCGCTGCCAGGTGCA
GTTTTACGGCCTGAGCGAAAACGATGAATGGACACAGGACAGGGCCAAG
CCCGTGACACAGATCGTGTCCGCCGAGGCATGGGGAAGGGCAGACTGT
GGCTTCACCTCCGAGTCTTACCAGCAGGGCGTGCTGAGCGCCACCATCC
TGTATGAGATCCTGCTGGGCAAGGCCACACTGTACGCCGTGCTGGTGTC
TGCCCTGGTGCTGATGGCCATGGTGAAGCGGAAGGATTCCAGAGGC

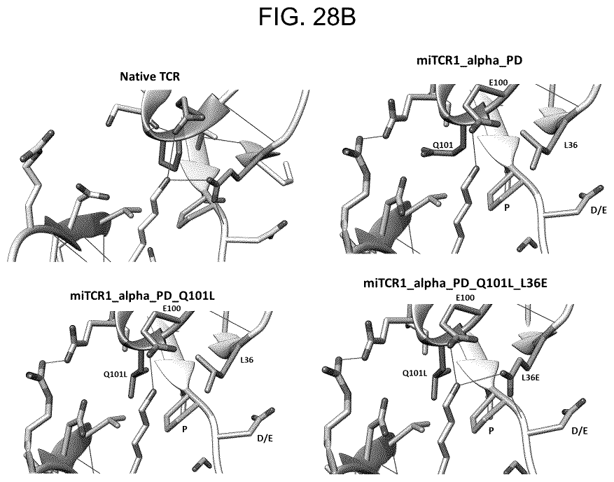

Transfection of the plasmids encoding these 16 (2 parental, 14 mutated) constructs into Jurkat cells revealed that many of the mutations improved surface expression as compared to the parent or wild-type constructs (see e.g., FIG. 29A), while some did not (e.g., JFC030, JFC036). These constructs were then converted to lentiviral vectors and primary human T cells were engineered. A similar pattern was observed in primary human T cells, wherein many of the modified miTCR constructs showed improved surface expression compared to the parent construct (see e.g., FIG. 29B). Of particular note is that each mutation had a consistent impact on expression. For example, PD insertion always improved expression in miTCR1, while G31K_P32F always reduced expression. Expression that is similar to the native TCR (1G4) was achieved for some constructs as a result of mutations, confirming that interface conflicts contribute to decreased surface expression, and resolution of interface conflicts improves expression.

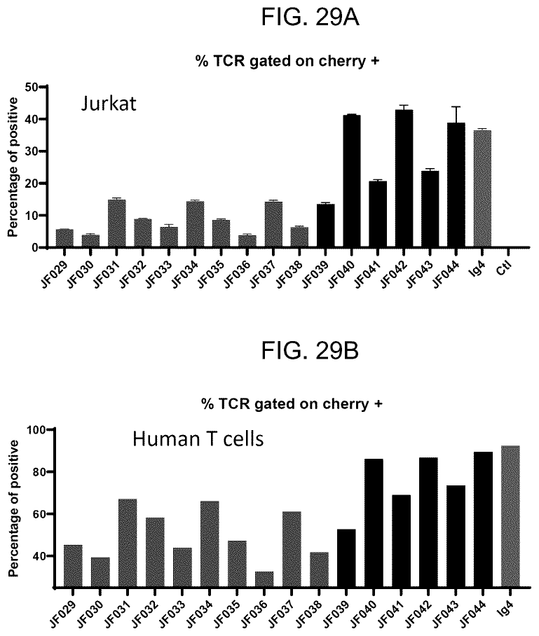

Next, it was important to demonstrate the function of miTCRs and that miTCRs are activated in an antigen-specific manner. Jurkat cells that contain a fluorescent reporter system and express miTCRs or CD28 and 41 BB-based CARs targeting CD19 were combined with either Nalm6 (CD19+) or Molm14 (CD19-negative). Activation of these reporters was only observed with CD19 (see e.g., FIG. 30). Thus, miTCRs are specifically activated by CD19 positive cells. Notably, miTCRs may activate more reporter activity than the CD28-based CAR.

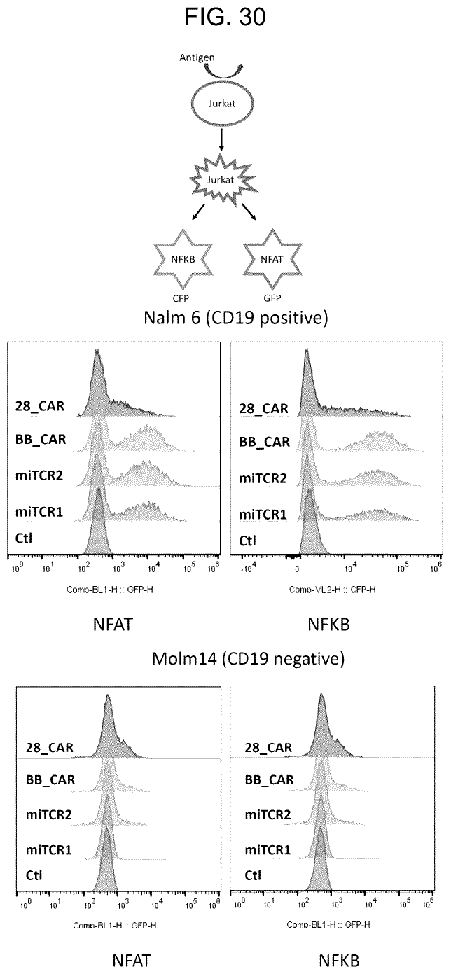

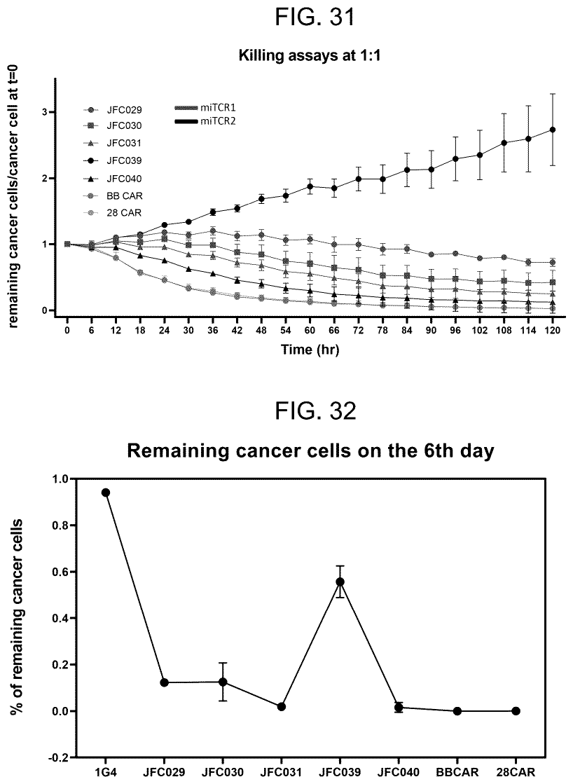

A few mutated miTCRs were selected to evaluate cytotoxic function against cancer cells. Parent constructs (JFC029, 039), CARs (BB and 28), and mutated constructs (JFC030, 031, 040) were combined with CD19+ Nalm6 cells and serially evaluated in co-culture for killing efficacy with primary human T cells. All mutants evaluated improved the function of their parental counterparts (see e.g., FIG. 31, Nalm6_wt). Of particular note is that JFC030, which was expressed at a lower level than its parent JFC029, also killed better than its parent. This suggests that the mutation actually improved function independently of expression. Focusing on the last time point of the killing assay, it was observed that miTCRs (except for JFC039, the original miTCR2) are more effective at killing targets than TCRs, and have efficacy similar to CARs (see e.g., FIG. 32, 1:1 Nalm6_wt).

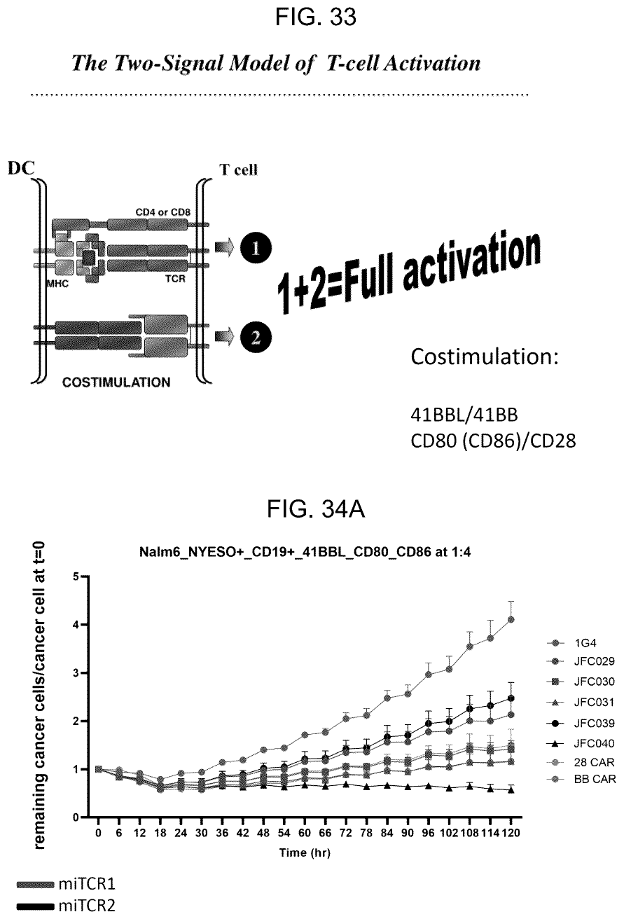

A notable distinction between miTCRs and CARs is that, like native TCRs, miTCRs do not have “built in” co-stimulation. Co-stimulation is thought to be required for full T cell activation, and is the principle behind why CARs contain a co-stimulatory domain (see e.g., FIG. 33). Of note, even without co-stimulation miTCRs are able to kill as efficiently as CARs (which do have co-stimulation).

With co-stimulation signal, JFC040 exhibits better effector function than CAR at lower effector (E) to target (T) ratio (E:T ratio). Target leukemia cells (Nalm6) were engineered to express co-stimulatory ligands and similar co-culture experiments were performed. In this context, miTCRs can (in the case of JFC040) kill better than CARs (see e.g., FIG. 34A) and expand better than CARs (see e.g., FIG. 34B). Both of these functional readouts are central predictors of function in patients.

Although the specific residue located at the interface for the V_{H} and V_{L} from different antibodies will be different, the key is to modify whatever interface residues from V_{H} and V_{L} in order to have sufficient contact with the interface residues from TCR constant region (highlighted in orange in FIG. 28B, for example).

1-88. (canceled)

89. A construct comprising an engineered chimeric MHC-independent T cell receptor (miTCR), the miTCR comprising:
A) a first peptide chain comprising:
i) a first TCR subunit comprising a constant region alpha chain (C_{α}) at least 80% identical to SEQ ID NO: 49; and
ii) a first antigen recognition variable region comprising an antigen recognition heavy chain (V_{H}) or an antigen recognition light chain (V_{L}); and
B) a second peptide chain comprising:
i) a second TCR subunit comprising a constant region beta chain (C_{β}) at least 80% identical to SEQ ID NO: 50; and
ii) a second antigen recognition variable region comprising an antigen recognition heavy chain (V_{H}) or an antigen recognition light chain (V_{L});
wherein the first peptide and second peptide are capable of self-assembling into a miTCR pair when expressed on a cell;
wherein the miTCR pair has an interface between the constant and variable regions; and
wherein the miTCR pair comprises an antigen recognition domain comprising the V_{H} and the V_{L} that together are capable of specifically binding to a target antigen.

90. The construct of claim 89, wherein the construct comprises one of:
V_{L}C_{α} and V_{H}C_{β}; or
V_{H}C_{α} and V_{L}C_{β}.

91. The construct of claim 89, wherein at least one of the first peptide chain and second peptide chain comprises human TCR constant regions.

92. The construct of claim 89, wherein the V_{H} is an antibody heavy chain and the V_{L} is an antibody light chain.

93. The construct of claim 89, wherein the target antigen is a virus-specific antigen selected from the group consisting of cytomegalovirus (CMV), Epstein-Barr Virus (EBV), Hepatitis B virus (HBV), Kaposi's sarcoma-associated herpes virus (KSHV), human papilloma virus (HPV), molluscum virus (MCV), human T-cell leukemia virus 1 (HTLV-1), HIV (human immunodeficiency virus), and hepatitis C virus (HCV).

94. The construct of claim 93, wherein the target antigen is a tumor-specific antigen selected from the group consisting of CD19, CD20, EGFR, Her2, PSCA, CD123, CEA (Carcinoembryonic Antigen), FAP, CD133, EGFRVIII, BCMA, PSMA, CA125, EphA2, C-met, L1CAM, VEGFR, CS1, ROR1, EC, NY-ESO-1, MUC1, MUC16, mesothelin, LewisY, GPC3, GD2, EPG, DLL3, CD99, 5T4, CD22, CD30, CD33, CD138, and CD171.

95. The construct of claim 89, wherein the antigen recognition domain targets antigens expressed on a cancer cell selected from the group consisting of adrenal cortex cancer, bladder cancer, breast cancer, cervical cancer, bile duct cancer, colorectal cancer, and esophageal cancer, Glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, Plasmacytoma, neuroblastoma, ovarian cancer, prostate cancer, sarcoma, gastric cancer, uterine cancer, and thyroid cancer.

96. The construct of claim 89, wherein the V_{L} comprises SEQ ID NO: 35 having at least one mutation selected from: Q101P, Q101L, Q101A, Q101G, Q101I, or Q101V; or L36E, L36D, L36K, L36R, L36N, and L36Q.

97. The construct of claim 89, wherein the V_{H} comprises SEQ ID NO: 36 having at least one mutation selected from: G31K, G31R, G31Q, P32F, P32L, P32V, P321, and P32A.

98. The construct of claim 89, wherein the V_{H} comprises SEQ ID NO: 36 having at least one mutation selected from T108L, T1081, T108M, and T108V.

99. The construct of claim 89, wherein the V_{H} comprises SEQ ID NO: 35 having at least one mutation selected from: S30K, S30R, S30H, S31Q, S31F, S31Y, S31H, and S31W.

100. The construct of claim 89, wherein the miTCR is selected from:
an FMC63 light chain variable region and a TCRα constant region;
an FMC63 heavy chain variable region and a TCRα constant region;
an FMC63 light chain variable region and a TCRβ constant region; and
an FMC63 heavy chain variable region and a TCRβ constant region.

101. The construct of claim 89, further comprising a co-stimulating domain selected from at least one of CD80(CD86)/CD28, 4-1 BBL/4-1 BBL, CD27, OX-40, ICOS, SLAM, CD84, CD30, GITR, and CD40L.

102. A pharmaceutical composition comprising the construct of claim 89 and a pharmaceutically acceptable carrier.

103. The pharmaceutical composition of claim 102, wherein the construct comprises one of:
V_{L}C_{α} and V_{H}C_{β}; or
V_{H}C_{α} and V_{L}C_{β}.

104. A method of treating a subject having cancer, the method comprising administering to the subject a pharmaceutical composition comprising the construct of claim 89.

105. The method of claim 104, wherein the construct comprises one of:
V_{L}C_{α} and V_{H}C_{β}; or
V_{H}C_{α} and V_{L}C_{β}.

106. The method of claim 104, wherein the target antigen is a tumor-specific antigen selected from the group consisting of CD19, CD20, EGFR, Her2, PSCA, CD123, CEA (Carcinoembryonic Antigen), FAP, CD133, EGFRVIII, BCMA, PSMA, CA125, EphA2, C-met, L1CAM, VEGFR, CS1, ROR1, EC, NY-ESO-1, MUC1, MUC16, mesothelin, LewisY, GPC3, GD2, EPG, DLL3, CD99, 5T4, CD22, CD30, CD33, CD138, and CD171.

107. The method of claim 104, wherein the antigen recognition domain targets antigens expressed on a cancer cell selected from the group consisting of adrenal cortex cancer, bladder cancer, breast cancer, cervical cancer, bile duct cancer, colorectal cancer, and esophageal cancer, Glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, Plasmacytoma, neuroblastoma, ovarian cancer, prostate cancer, sarcoma, gastric cancer, uterine cancer, and thyroid cancer.

108. The method of claim 104, wherein the miTCR is selected from:
an FMC63 light chain variable region and a TCRα constant region;
an FMC63 heavy chain variable region and a TCRα constant region;
an FMC63 light chain variable region and a TCRβ constant region; and
an FMC63 heavy chain variable region and a TCRβ constant region.


SEQUENCE LISTING (50 sequences)

SEQ ID NO: 1 - DNA, 2674 bp, Artificial Sequence
  synthetic miTCR construct
atggagaccctgctgggcctgctgatcctgtggctgcagctgcagtgggtgagctccaagcaggacatccagatgacccagaccacatctagcctgtctgccagcctgggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagcaggaggatatcgccacctatttttgtcagcagggcaataccctgccttacacattcggcggcggcaccaagctggagatcacaatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccggccccggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatggaagcggagctactaacttcagcctgctgaagcaggctggagacgtggaggagaaccctggacctgtttctgaagccatggtgtctaagggcgaggaggataatatggccatcatcaaggagttcatgaggtttaaggtgcacatggagggcagcgtgaacggccacgagtttgagatcgagggagagggagagggccgcccatacgagggaacccagacagccaagctgaaggtgaccaagggcggccctctgccttttgcctgggacatcctgtcccctcagttcatgtacggctctaaggcctatgtgaagcacccagccgacatccccgattacctgaagctgtccttcccagagggctttaagtgggagagagtgatgaacttcgaggatggcggcgtggtgaccgtgacacaggattctagcctgcaggacggcgagtttatctacaaggtgaagctgagaggcaccaatttccctagcgacggcccagtgatgcagaagaagacaatgggctgggaggcctcctctgagaggatgtatcctgaggatggcgccctgaagggcgagatcaagcagcgcctgaagctgaaggatggcggccactacgacgccgaggtgaagaccacatataaggccaagaagccagtgcagctgcctggagcctacaacgtgaatatcaagctggatatcaccagccacaatgaggactacacaatcgtggagcagtatgagagggcagagggcagacactccaccggaggcatggacgagctgtataagtaagcggccgccacccagctttcttgtacaaagtggt

SEQ ID NO: 2 - DNA, 2642 bp, Artificial Sequence
  synthetic miTCR construct
atggagaccctgctgggcctgctgatcctgtggctgcagctgcagtgggtgagctccaagcaggaggtgaagctgcaggagagcggccccggactggtggcaccttctcagagcctgtccgtgacctgcacagtgagcggcgtgtccctgcctgactacggcgtgagctggatcaggcagccacctagaaagggactggagtggctgggcgtgatctggggctccgagaccacatactataactccgccctgaagtctcggctgaccatcatcaaggataactctaagagccaggtgtttctgaagatgaattccctgcagaccgacgatacagccatctactattgtgccaagcactactattacggcggctcttatgccatggactactggggccagggcaccagcgtgacagtgtctagcatccagaaccctgatccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggtctaataagagcgacttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggcgtggacatccagatgacccagaccacatcctctctgtctgccagcctgggcgacagggtgacaatcagctgccgcgcctcccaggatatctctaagtatctgaattggtaccagcagaagccagatggcaccgtgaagctgctgatctatcacacaagccggctgcactccggagtgccatctagattttccggatctggaagcggaaccgactacagcctgacaatctccaacctggagcaggaggatatcgccacctatttttgccagcagggcaataccctgccctacacattcggcggcggcaccaagctggagatcacagacctgaagaacgtgttcccacccgaggtggccgtgtttgagccttctgaggccgagatcagccacacccagaaggccaccctggtgtgcctggcaaccggcttctatccagatcacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatggaagcggagctactaacttcagcctgctgaagcaggctggagacgtggaggagaaccctggacctgtttctgaagccatggtgtctaagggcgaggaggataatatggccatcatcaaggagttcatgaggtttaaggtgcacatggagggcagcgtgaacggccacgagtttgagatcgagggagagggagagggccgcccatacgagggaacccagacagccaagctgaaggtgaccaagggcggccctctgccttttgcctgggacatcctgtcccctcagttcatgtacggctctaaggcctatgtgaagcacccagccgacatccccgattacctgaagctgtccttcccagagggctttaagtgggagagagtgatgaacttcgaggatggcggcgtggtgaccgtgacacaggattctagcctgcaggacggcgagtttatctacaaggtgaagctgagaggcaccaatttccctagcgacggcccagtgatgcagaagaagacaatgggctgggaggcctcctctgagaggatgtatcctgaggatggcgccctgaagggcgagatcaagcagcgcctgaagctgaaggatggcggccactacgacgccgaggtgaagaccacatataaggccaagaagccagtgcagctgcctggagcctacaacgtgaatatcaagctggatatcaccagccacaatgaggactacacaatcgtggagcagtatgagagggcagagggcagacactccaccggaggcatggacgagctgtataagtaagc

SEQ ID NO: 3 - DNA, 1875 bp, Artificial Sequence
  synthetic miTCR construct
gtcgacgttaacgccgccaccatggagaccctgctgggcctgctgatcctgtggctgcagctgcagtgggtgagctccaagcaggacatccagatgacccagaccacatctagcctgtctgccagcctgggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagcaggaggatatcgccacctatttttgtcagcagggcaataccctgccttacacattcggcggcggcaccaagctggagatcacaatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccaagttcggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatgga

SEQ ID NO: 4 - DNA, 1881 bp, Artificial Sequence
  synthetic miTCR construct
gtcgacgttaacgccgccaccatggagaccctgctgggcctgctgatcctgtggctgcagctgcagtgggtgagctccaagcaggacatccagatgacccagaccacatctagcctgtctgccagcctgggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagcaggaggatatcgccacctatttttgtcagcagggcaataccctgccttacacattcggcggcggcaccaagctggagatcacacccgacatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccggccccggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatgga

SEQ ID NO: 5 - DNA, 1881 bp, Artificial Sequence
  synthetic miTCR construct
gtcgacgttaacgccgccaccatggagaccctgctgggcctgctgatcctgtggctgcagctgcagtgggtgagctccaagcaggacatccagatgacccagaccacatctagcctgtctgccagcctgggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagcaggaggatatcgccacctatttttgtcagcagggcaataccctgccttacacattcggcggcggcaccaagctggagatcacacccgacatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccaagttcggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatgga

SEQ ID NO: 6 - DNA, 1875 bp, Artificial Sequence
  synthetic miTCR construct
gtcgacgttaacgccgccaccatggagaccctgctgggcctgctgatcctgtggctgcagctgcagtgggtgagctccaagcaggacatccagatgacccagaccacatctagcctgtctgccagcctgggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagctggaggatatcgccacctatttttgtcagcagggcaataccctgccttacacattcggcggcggcaccaagctggagatcacaatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccggccccggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatgga

SEQ ID NO: 7 - DNA, 1878 bp, Artificial Sequence
  synthetic miTCR construct
gtcgacgttaacgccgccaccatggagaccctgctgggcctgctgatcctgtggctgcagctgcagtgggtgagctccaagcaggacatccagatgacccagaccacatctagcctgtctgccagcctgggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagctggaggatatcgccacctatttttgtcagcagggcaataccctgccttacacattcggcggcggcaccaagctggagatcacacccgacatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccggccccggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgat

SEQ ID NO: 8 - DNA, 1878 bp, Artificial Sequence
  synthetic miTCR construct
gtcgacgttaacgccgccaccatggagaccctgctgggcctgctgatcctgtggctgcagctgcagtgggtgagctccaagcaggacatccagatgacccagaccacatctagcctgtctgccagcgagggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagctggaggatatcgccacctatttttgtcagcagggcaataccctgccttacacattcggcggcggcaccaagctggagatcacacccgacatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccggccccggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgat

SEQ ID NO: 9 - DNA, 1925 bp, Artificial Sequence
  synthetic miTCR construct
gtcgacgttaacgccgccaccatggagaccctgctgggcctgctgatcctgtggctgcagctgcagtgggtgagctccaagcaggacatccagatgacccagaccacatctagcctgtctgccagcctgggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagctggaggatatcgccacctatttttgtcagcagggcaataccctgccttacacattcggcggcggcaccaagctggagatcacaatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccaagttcggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatggaagcggagctactaacttcagcctgctgaagcaggctggagacgtggagga

SEQ ID NO: 10 - DNA, 1879 bp, Artificial Sequence
  synthetic miTCR construct
gtcgacgttaacgccgccaccatggagaccctgctgggcctgctgatcctgtggctgcagctgcagtgggtgagctccaagcaggacatccagatgacccagaccacatctagcctgtctgccagcctgggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagctggaggatatcgccacctatttttgtcagcagggcaataccctgccttacacattcggcggcggcaccaagctggagatcacacccgacatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccaagttcggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatg

SEQ ID NO: 11 - DNA, 1879 bp, Artificial Sequence
  synthetic miTCR construct
gtcgacgttaacgccgccaccatggagaccctgctgggcctgctgatcctgtggctgcagctgcagtgggtgagctccaagcaggacatccagatgacccagaccacatctagcctgtctgccagcgagggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagctggaggatatcgccacctatttttgtcagcagggcaataccctgccttacacattcggcggcggcaccaagctggagatcacacccgacatccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggagtggaggtgaagctgcaggagtccaagttcggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctctgatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatg

SEQ ID NO: 12 - DNA, 1875 bp, Artificial Sequence
  synthetic miTCR construct
gtcgacgttaacgccgccaccatggagaccctgctgggcctgctgatcctgtggctgcagctgcagtgggtgagctccaagcaggaggtgaagctgcaggagagcggccccggactggtggcaccttctcagagcctgtccgtgacctgcacagtgagcggcgtgtccctgcctgactacggcgtgagctggatcaggcagccacctagaaagggactggagtggctgggcgtgatctggggctccgagaccacatactataactccgccctgaagtctcggctgaccatcatcaaggataactctaagagccaggtgtttctgaagatgaattccctgcagaccgacgatacagccatctactattgtgccaagcactactattacggcggctcttatgccatggactactggggccagggcaccagcgtgacagtgtctagcatccagaaccctgatccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggtctaataagagcgacttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggcgtggacatccagatgacccagaccacaaagttcctgtctgccagcctgggcgacagggtgacaatcagctgccgcgcctcccaggatatctctaagtatctgaattggtaccagcagaagccagatggcaccgtgaagctgctgatctatcacacaagccggctgcactccggagtgccatctagattttccggatctggaagcggaaccgactacagcctgacaatctccaacctggagcaggaggatatcgccacctatttttgccagcagggcaataccctgccctacacattcggcggcggcaccaagctggagatcacagacctgaagaacgtgttcccacccgaggtggccgtgtttgagccttctgaggccgagatcagccacacccagaaggccaccctggtgtgcctggcaaccggcttctatccagatcacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatgga

SEQ ID NO: 13 - DNA, 1878 bp, Artificial Sequence
  synthetic miTCR construct
gtcgacgttaacgccgccaccatggagaccctgctgggcctgctgatcctgtggctgcagctgcagtgggtgagctccaagcaggaggtgaagctgcaggagagcggccccggactggtggcaccttctcagagcctgtccgtgacctgcacagtgagcggcgtgtccctgcctgactacggcgtgagctggatcaggcagccacctagaaagggactggagtggctgggcgtgatctggggctccgagaccacatactataactccgccctgaagtctcggctgaccatcatcaaggataactctaagagccaggtgtttctgaagatgaattccctgcagaccgacgatacagccatctactattgtgccaagcactactattacggcggctcttatgccatggactactggggccagggcaccagcgtgacagtgtctcccgacatccagaaccctgatccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggtctaataagagcgacttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggcgtggacatccagatgacccagaccacatcctctctgtctgccagcctgggcgacagggtgacaatcagctgccgcgcctcccaggatatctctaagtatctgaattggtaccagcagaagccagatggcaccgtgaagctgctgatctatcacacaagccggctgcactccggagtgccatctagattttccggatctggaagcggaaccgactacagcctgacaatctccaacctggagcaggaggatatcgccacctatttttgccagcagggcaataccctgccctacacattcggcggcggcaccaagctggagatcacagacctgaagaacgtgttcccacccgaggtggccgtgtttgagccttctgaggccgagatcagccacacccagaaggccaccctggtgtgcctggcaaccggcttctatccagatcacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatgga

SEQ ID NO: 14 - DNA, 1878 bp, Artificial Sequence
  synthetic miTCR construct
gtcgacgttaacgccgccaccatggagaccctgctgggcctgctgatcctgtggctgcagctgcagtgggtgagctccaagcaggaggtgaagctgcaggagagcggccccggactggtggcaccttctcagagcctgtccgtgacctgcacagtgagcggcgtgtccctgcctgactacggcgtgagctggatcaggcagccacctagaaagggactggagtggctgggcgtgatctggggctccgagaccacatactataactccgccctgaagtctcggctgaccatcatcaaggataactctaagagccaggtgtttctgaagatgaattccctgcagaccgacgatacagccatctactattgtgccaagcactactattacggcggctcttatgccatggactactggggccagggcaccagcgtgacagtgtctcccgacatccagaaccctgatccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggtctaataagagcgacttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggcgtggacatccagatgacccagaccacaaagttcctgtctgccagcctgggcgacagggtgacaatcagctgccgcgcctcccaggatatctctaagtatctgaattggtaccagcagaagccagatggcaccgtgaagctgctgatctatcacacaagccggctgcactccggagtgccatctagattttccggatctggaagcggaaccgactacagcctgacaatctccaacctggagcaggaggatatcgccacctatttttgccagcagggcaataccctgccctacacattcggcggcggcaccaagctggagatcacagacctgaagaacgtgttcccacccgaggtggccgtgtttgagccttctgaggccgagatcagccacacccagaaggccaccctggtgtgcctggcaaccggcttctatccagatcacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccgatgga

SEQ ID NO: 15 - DNA, 1873 bp, Artificial Sequence
  synthetic miTCR construct
gtcgacgttaacgccgccaccatggagaccctgctgggcctgctgatcctgtggctgcagctgcagtgggtgagctccaagcaggaggtgaagctgcaggagagcggccccggactggtggcaccttctcagagcctgtccgtgacctgcacagtgagcggcgtgtccctgcctgactacggcgtgagctggatcaggcagccacctagaaagggactggagtggctgggcgtgatctggggctccgagaccacatactataactccgccctgaagtctcggctgaccatcatcaaggataactctaagagccaggtgtttctgaagatgaattccctgcagctggacgatacagccatctactattgtgccaagcactactattacggcggctcttatgccatggactactggggccagggcaccagcgtgacagtgtctcccgacatccagaaccctgatccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggtctaataagagcgacttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggcgtggacatccagatgacccagaccacatcctctctgtctgccagcctgggcgacagggtgacaatcagctgccgcgcctcccaggatatctctaagtatctgaattggtaccagcagaagccagatggcaccgtgaagctgctgatctatcacacaagccggctgcactccggagtgccatctagattttccggatctggaagcggaaccgactacagcctgacaatctccaacctggagcaggaggatatcgccacctatttttgccagcagggcaataccctgccctacacattcggcggcggcaccaagctggagatcacagacctgaagaacgtgttcccacccgaggtggccgtgtttgagccttctgaggccgagatcagccacacccagaaggccaccctggtgtgcctggcaaccggcttctatccagatcacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccg

SEQ ID NO: 16 - DNA, 1874 bp, Artificial Sequence
  synthetic miTCR construct
gtcgacgttaacgccgccaccatggagaccctgctgggcctgctgatcctgtggctgcagctgcagtgggtgagctccaagcaggaggtgaagctgcaggagagcggccccggactggtggcaccttctcagagcctgtccgtgacctgcacagtgagcggcgtgtccctgcctgactacggcgtgagctggatcaggcagccacctagaaagggactggagtggctgggcgtgatctggggctccgagaccacatactataactccgccctgaagtctcggctgaccatcatcaaggataactctaagagccaggtgtttctgaagatgaattccctgcagctggacgatacagccatctactattgtgccaagcactactattacggcggctcttatgccatggactactggggccagggcaccagcgtgacagtgtctcccgacatccagaaccctgatccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggtctaataagagcgacttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagcagggccaagcgcggcagcggcgagggcagaggaagtctgctgacatgcggagacgtggaggagaatcctggacctatgtccatcggactgctgtgctgtgccgccctgagcctgctgtgggccggcccagtgaacgcaggcgtggacatccagatgacccagaccacaaagttcctgtctgccagcctgggcgacagggtgacaatcagctgccgcgcctcccaggatatctctaagtatctgaattggtaccagcagaagccagatggcaccgtgaagctgctgatctatcacacaagccggctgcactccggagtgccatctagattttccggatctggaagcggaaccgactacagcctgacaatctccaacctggagcaggaggatatcgccacctatttttgccagcagggcaataccctgccctacacattcggcggcggcaccaagctggagatcacagacctgaagaacgtgttcccacccgaggtggccgtgtttgagccttctgaggccgagatcagccacacccagaaggccaccctggtgtgcctggcaaccggcttctatccagatcacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggccggaccga

SEQ ID NO: 17 - protein, 618 aa, Artificial Sequence
  synthetic miTCR
METLLGLLILWLQLQWVSSKQDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDG

SEQ ID NO: 18 - protein, 618 aa, Artificial Sequence
  synthetic miTCR
METLLGLLILWLQLQWVSSKQEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDG

SEQ ID NO: 19 - protein, 890 aa, Artificial Sequence
  synthetic miTCR
METLLGLLILWLQLQWVSSKQDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESKFGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKTAAGCGGCCGC

SEQ ID NO: 20 - protein, 881 aa, Artificial Sequence
  synthetic miTCR
METLLGLLILWLQLQWVSSKQDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 21 - protein, 881 aa, Artificial Sequence
  synthetic miTCR
METLLGLLILWLQLQWVSSKQDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESKFGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 22 - protein, 879 aa, Artificial Sequence
  synthetic miTCR
METLLGLLILWLQLQWVSSKQDIQMTQTTSSLSASLGDRVTISCRASQDISKYLNWYQQKPDGTVKLLIYHTSRLHSGVPSRFSGSGSGTDYSLTISNLELEDIATYFCQQGNTLPYTFGGGTKLEITIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 23 - protein, 881 aa, Artificial Sequence
  synthetic miTCR
METLLGLLILWLQLQWVSSKQDIQMTQTTSSLSASLGDRVTISCRASQDISKYLNWYQQKPDGTVKLLIYHTSRLHSGVPSRFSGSGSGTDYSLTISNLELEDIATYFCQQGNTLPYTFGGGTKLEITPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 24 - protein, 881 aa, Artificial Sequence
  synthetic miTCR
METLLGLLILWLQLQWVSSKQDIQMTQTTSSLSASEGDRVTISCRASQDISKYLNWYQQKPDGTVKLLIYHTSRLHSGVPSRFSGSGSGTDYSLTISNLELEDIATYFCQQGNTLPYTFGGGTKLEITPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 25 - protein, 879 aa, Artificial Sequence
  synthetic miTCR
METLLGLLILWLQLQWVSSKQDIQMTQTTSSLSASLGDRVTISCRASQDISKYLNWYQQKPDGTVKLLIYHTSRLHSGVPSRFSGSGSGTDYSLTISNLELEDIATYFCQQGNTLPYTFGGGTKLEITIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESKFGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 26 - protein, 881 aa, Artificial Sequence
  synthetic miTCR
METLLGLLILWLQLQWVSSKQDIQMTQTTSSLSASLGDRVTISCRASQDISKYLNWYQQKPDGTVKLLIYHTSRLHSGVPSRFSGSGSGTDYSLTISNLELEDIATYFCQQGNTLPYTFGGGTKLEITPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESKFGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 27 - protein, 881 aa, Artificial Sequence
  synthetic miTCR
METLLGLLILWLQLQWVSSKQDIQMTQTTSSLSASEGDRVTISCRASQDISKYLNWYQQKPDGTVKLLIYHTSRLHSGVPSRFSGSGSGTDYSLTISNLELEDIATYFCQQGNTLPYTFGGGTKLEITPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVEVKLQESKFGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 28 - protein, 879 aa, Artificial Sequence
  synthetic miTCR
METLLGLLILWLQLQWVSSKQEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVDIQMTQTTKFLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 29 - protein, 880 aa, Artificial Sequence
  synthetic miTCR
METLLGLLILWLQLQWVSSKQEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 30 - protein, 880 aa, Artificial Sequence
  synthetic miTCR
METLLGLLILWLQLQWVSSKQEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVDIQMTQTTKFLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 31 - protein, 880 aa, Artificial Sequence
  synthetic miTCR
METLLGLLILWLQLQWVSSKQEVKLQESGPGLVAPSQSLSVTCTVSGVSLPDYGVSWIRQPPRKGLEWLGVIWGSETTYYNSALKSRLTIIKDNSKSQVFLKMNSLQLDDTAIYYCAKHYYYGGSYAMDYWGQGTSVTVSPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 32 - protein, 880 aa, Artificial Sequence
  synthetic miTCR
METLLGLLILWLQLQWVSSKQEVKLQESGPGLVAPSQSLSVTCTVSGVSLPDYGVSWIRQPPRKGLEWLGVIWGSETTYYNSALKSRLTIIKDNSKSQVFLKMNSLQLDDTAIYYCAKHYYYGGSYAMDYWGQGTSVTVSPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSSRAKRGSGEGRGSLLTCGDVEENPGPMSIGLLCCAALSLLWAGPVNAGVDIQMTQTTKFLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRTDGSGATNFSLLKQAGDVEENPGPVSEAMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 33 - protein, 140 aa, Artificial Sequence
  synthetic miTCR
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 34 - protein, 178 aa, Artificial Sequence
  synthetic miTCR
DLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 35 - protein, 128 aa, Artificial Sequence
  synthetic miTCR
METLLGLLILWLQLQWVSSKQDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 36 - protein, 143 aa, Artificial Sequence
  synthetic miTCR
MSIGLLCCAALSLLWAGPVNAGVEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 37 - DNA, 384 bp, Artificial Sequence
  synthetic miTCR construct
atggagaccctgctgggcctgctgatcctgtggctgcagctgcagtgggtgagctccaagcaggacatccagatgacccagaccacatctagcctgtctgccagcctgggcgatagggtgacaatctcctgccgcgcctctcaggacatcagcaagtatctgaactggtaccagcagaagccagatggcaccgtgaagctgctgatctatcacacatcccggctgcactctggcgtgcccagcagattttccggatctggaagcggaaccgactactccctgacaatctctaacctggagcaggaggatatcgccacctatttttgtcagcagggcaataccctgccttacacattcggcggcggcaccaagctggagatcaca

SEQ ID NO: 38 - DNA, 429 bp, Artificial Sequence
  synthetic miTCR construct
atgtccatcggactgctgtgctgtgccgccctgagcctgctgtgggccggcccagtgaacgcaggagtggaggtgaagctgcaggagtccggccccggactggtggcaccttctcagagcctgtccgtgacctgcacagtgtctggcgtgagcctgccagactacggcgtgagctggatcaggcagccacctagaaagggactggagtggctgggcgtgatctggggctccgagaccacatactataatagcgccctgaagtcccggctgaccatcatcaaggataactctaagagccaggtgtttctgaagatgaatagcctgcagaccgacgatacagccatctactattgcgccaagcactactattacggcggctcttatgccatggactactggggccagggcaccagcgtgacagtgtcctct

SEQ ID NO: 39 - DNA, 420 bp, Artificial Sequence
  synthetic miTCR construct
atccagaaccctgacccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggagcaataagtccgatttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagc

SEQ ID NO: 40 - DNA, 534 bp, Artificial Sequence
  synthetic miTCR construct
gatctgaagaacgtgttcccacccgaggtggccgtgtttgagccttctgaggccgagatcagccacacccagaaggccaccctggtgtgcctggcaaccggcttctatccagaccacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggc

SEQ ID NO: 41 - DNA, 390 bp, Artificial Sequence
  synthetic miTCR construct
atgtccatcggactgctgtgctgtgccgccctgagcctgctgtgggccggcccagtgaacgcaggcgtggacatccagatgacccagaccacatcctctctgtctgccagcctgggcgacagggtgacaatcagctgccgcgcctcccaggatatctctaagtatctgaattggtaccagcagaagccagatggcaccgtgaagctgctgatctatcacacaagccggctgcactccggagtgccatctagattttccggatctggaagcggaaccgactacagcctgacaatctccaacctggagcaggaggatatcgccacctatttttgccagcagggcaataccctgccctacacattcggcggcggcaccaagctggagatcaca

SEQ ID NO: 42 - DNA, 423 bp, Artificial Sequence
  synthetic miTCR construct
atggagaccctgctgggcctgctgatcctgtggctgcagctgcagtgggtgagctccaagcaggaggtgaagctgcaggagagcggccccggactggtggcaccttctcagagcctgtccgtgacctgcacagtgagcggcgtgtccctgcctgactacggcgtgagctggatcaggcagccacctagaaagggactggagtggctgggcgtgatctggggctccgagaccacatactataactccgccctgaagtctcggctgaccatcatcaaggataactctaagagccaggtgtttctgaagatgaattccctgcagaccgacgatacagccatctactattgtgccaagcactactattacggcggctcttatgccatggactactggggccagggcaccagcgtgacagtgtctagc

SEQ ID NO: 43 - DNA, 420 bp, Artificial Sequence
  synthetic miTCR construct
atccagaaccctgatccagccgtgtatcagctgagggactccaagtcctctgataagagcgtgtgcctgttcaccgactttgattctcagacaaacgtgagccagtctaaggacagcgacgtgtacatcaccgacaagtgcgtgctggatatgcgcagcatggacttcaagagcaactccgccgtggcctggtctaataagagcgacttcgcctgcgccaacgcctttaacaattccatcatcccagaggatacattctttccctctcctgagagctcctgtgacgtgaagctggtggagaagagcttcgagaccgatacaaacctgaattttcagaacctgtccgtgatcggcttccggatcctgctgctgaaggtggccggctttaatctgctgatgaccctgagactgtggtctagc

SEQ ID NO: 44 - DNA, 534 bp, Artificial Sequence
  synthetic miTCR construct
gacctgaagaacgtgttcccacccgaggtggccgtgtttgagccttctgaggccgagatcagccacacccagaaggccaccctggtgtgcctggcaaccggcttctatccagatcacgtggagctgagctggtgggtgaacggcaaggaggtgcactccggcgtgtgcacagaccctcagccactgaaggagcagcccgccctgaatgattcccggtattgtctgagctcccggctgagagtgtctgccaccttttggcagaaccctaggaatcacttccgctgccaggtgcagttttacggcctgagcgaaaacgatgaatggacacaggacagggccaagcccgtgacacagatcgtgtccgccgaggcatggggaagggcagactgtggcttcacctccgagtcttaccagcagggcgtgctgagcgccaccatcctgtatgagatcctgctgggcaaggccacactgtacgccgtgctggtgtctgccctggtgctgatggccatggtgaagcggaaggattccagaggc

SEQ ID NO: 45 - DNA, 20 bp, Artificial Sequence
  synthetic miTCR construct
cgtgctggatatgcgcagca

SEQ ID NO: 46 - DNA, 20 bp, Artificial Sequence
  synthetic miTCR construct
cgaaaacgatgaatggacac

SEQ ID NO: 47 - DNA, 20 bp, Artificial Sequence
  synthetic guide
tgtgctagacatgaggtcta

SEQ ID NO: 48 - DNA, 20 bp, Artificial Sequence
  synthetic guide
ggagaatgacgagtggaccc

SEQ ID NO: 49 - protein, 140 aa, Artificial Sequence
  synthetic miTCR
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 50 - protein, 178 aa, Artificial Sequence
  synthetic miTCR
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG